(19) United States
(12) Patent Application Publication
(10) Pub. No.: US 2008/0015214 A1
(43) Pub. Date: Jan. 17, 2008

(54) 3-PIPERIDIN-4-YL-INDOLE ORL-1 RECEPTOR MODULATORS

(76) Inventors: Gilles C. Bignan, Bridgewater, NJ (US); Kathleen A. Battista, Williamstown, NJ (US); Peter J. Connolly, New Providence, NJ (US); Jessica J. Liu, Three Bridges, NJ (US); Steven A. Middleton, Flemington, NJ (US); Michael J. Orsini, Somerset, NJ (US); Allen B. Reitz, Lansdale, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/550,421

(22) Filed: Oct. 18, 2006

Related U.S. Application Data
(60) Provisional application No. 60/729,766, filed on Oct. 24, 2005.

(51) Int. Cl. A61K 31/454; A61K 31/437; A61K 31/4545; A61P 1/00; A61P 25/00; A61P 9/00; C07D 401/02; C07D 401/14; C07D 471/02

(52) U.S. Cl. 514/300; 514/318; 514/323; 546/113; 546/194; 546/201

(57) ABSTRACT

The present invention is directed to novel 3-piperidin-4-yl-indole derivatives of formula (I) 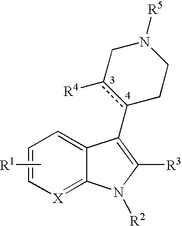
and forms thereof, wherein X, R¹, R², R³, R⁴ and R⁵ are as herein defined, pharmaceutical compositions thereof and use as ORL-1 receptor modulators for treating, preventing or ameliorating ORL-1 receptor mediated disorders and conditions.

### CROSS REFERENCE TO RELATED APPLICATIONS

This present application claims benefit of U.S. Provisional Patent Application Ser. No. 60/729,766 filed Oct. 24, 2005, which is incorporated herein by reference in its entirety and for all purposes.

### FIELD OF THE INVENTION

The present invention is directed to novel 3-piperidin-4-yl-indole derivatives, pharmaceutical compositions containing them and their use in the treating, preventing or ameliorating ORL-1 receptor mediated disorders and conditions.

### BACKGROUND OF THE INVENTION

The ORL-1 (orphan opioid receptor) G-protein coupled receptor was first reported in 1994, and was discovered based on its homology with the classic opioid receptors: OP-1, OP-3 and OP-2 (delta, mu and kappa, respectively). Also known as the nociceptin receptor, the ORL-1 G-protein coupled receptor does not bind opioid ligands with high affinity. The amino acid sequence of ORL-1 is 47% identical to the opioid receptors overall and 64% identical in the transmembrane domains. (Nature, 1995, 377, 532.)

The endogenous ORL-1 ligand, known as nociceptin, is a highly basic 17 amino acid peptide that was isolated from tissue extracts in 1995. The ligand was named nociceptin because sensitivity to pain was increased when the ligand was injected into mouse brain. The ligand was also named orphanin FQ (OFQ) because terminal phenylalanine (F) and glutamine (Q) residues flank the peptide on the N- and C-termini respectively (see PCT application, WO 97/07212).

Nociceptin binding to ORL-1 receptors causes inhibition of cAMP synthesis, inhibition of voltage-gated calcium channels and activation of potassium conductance. Nociceptin produces a variety of in vivo pharmacological effects which, at times, oppose the effects of opioids, including hyperalgesia and inhibition of morphine-induced analgesia. Mutant mice lacking nociceptin receptors show better performance in learning and memory tasks. These mutant mice also have normal responses to painful stimuli.

The ORL-1 receptor is widely distributed/expressed throughout the human body, including in the brain and spinal cord. The ORL-1 receptor exists in both the dorsal and ventral horns of the spinal cord. Precursor ORL-1 mRNA has been found in the superficial lamina of the dorsal horn, where primary afferent fibers of nociceptors terminate. Therefore, the ORL-1 has an important role in nociception transmission in the spinal cord. Recent studies have confirmed that nociceptin, when given to mice by i.c.v. injection, induces hyperalgesia and decreases locomotor activity. (Brit. J. Pharmacol. 2000, 129, 1261.)

There remains a need, therefore, for small molecule ORL-1 modulators for treating, preventing or ameliorating ORL-1 receptor mediated disorders and conditions such as, but not limited to, anxiety, depression, panic, dementia, mania, bipolar disorder, substance abuse, neuropathic pain, acute pain, chronic pain, migraine, asthma, cough, psychosis, schizophrenia, epilepsy, hypertension, obesity, eating disorders, cravings, diabetes, cardiac arrhythmia, irritable bowel syndrome, Crohn's disease, urinary incontinence, adrenal disorders, attention deficit disorder (ADD), attention deficit hyperactivity disorders (ADHD), Alzheimer's disease, for improved cognition or memory and for mood stabilization (Bignan G C, Connolly P J, Middleton S A, Recent advances towards the discovery of ORL-1 receptor agonists and antagonists, Expert Opinion on Therapeutic Patents, 2005, 15(4), 357-388).

Procedures described in PCT Applications WO 02020013 and WO 02014317 were used to prepare certain 3-piperidin-4-yl-indole intermediate compounds described herein.

### SUMMARY OF THE INVENTION

The present invention is directed to 3-piperidin-4-yl-indole derivatives of formula (I)

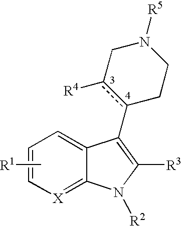

and forms thereof, wherein X, R¹, R², R³, R⁴ and R⁵ are as herein defined.

The modulator compounds of the present invention are useful as agonists, inverse agonists or antagonists of the ORL-1 receptor. The compounds of formula (I) are useful as modulators for treating, preventing or ameliorating ORL-1 receptor mediated disorders and conditions.

The compounds of formula (I) further include intermediates useful for the synthesis of additional representative compounds of the present invention.

The present invention is also directed to a method for treating, preventing or ameliorating ORL-1 receptor mediated disorders and conditions in a subject in need thereof comprising administering to the subject an effective amount of a compound of formula (I).

### DETAILED DESCRIPTION OF THE INVENTION

The present invention is directed to compounds of formula (I) 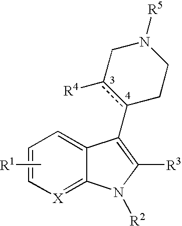
and forms thereof, wherein
the dashed line between position 3 and 4 in formula (I) represents a location for an optionally present double bond;
X is selected from the group consisting of CH and N;
R¹ is hydrogen or one, two, three or four substituents each selected from the group consisting of C₁₋₈alkyl, C₁₋₈alkoxy, amino, amino-C₁₋₈alkyl, halogen, C₁₋₈alkyl-halo, C₁₋₈alkoxy-halo, hydroxy, cyano and nitro;
R² is selected from the group consisting of hydrogen, C₃₋₁₄cycloalkyl and C₁₋₈alkyl; optionally substituted on C₁₋₈alkyl with one, two or three substituents each selected from the group consisting of C₁₋₈alkoxy, C₁₋₈acyl, oxy-C₁₋₈acyl, amino, amino-C₁₋₈alkyl, halogen, C₁₋₈alkyl-halo, hydroxy, carbonyl-C₁₋₈alkoxy, aryl, oxy-aryl, heterocyclyl, oxy-heterocyclyl, C₃₋₁₄cycloalkyl and oxy-C₃₋₁₄cycloalkyl;
R³ is selected from the group consisting of hydrogen and C₁₋₈alkyl;
R⁴ is one substituent when the double bond between position 3 and 4 in formula (I) is present or two substituents when the double bond between position 3 and 4 in formula (I) is not present, wherein each substituent is each selected from the group consisting of hydrogen, hydroxy and oxy-C₁₋₈acyl; and
R⁵ is selected from the group consisting of hydrogen, C₁₋₈alkyl, C₁₋₈acyl, carbonyl-C₁₋₈alkoxy, heterocyclyl, C₁₋₈acyl-heterocyclyl, C₃₋₁₄cycloalkyl, C₁₋₈alkyl-C₃₋₁₄cycloalkyl, carbonyl-C₃₋₁₄cycloalkyl, aryl and C₁₋₈alkyl-aryl,
wherein carbonyl-C₃₋₁₄cycloalkyl is optionally substituted on C₃₋₁₄cycloalkyl with one or two C₁₋₈alkyl substituents,
wherein C₁₋₈alkyl-aryl is optionally substituted on aryl with one, two or three substituents each selected from the group consisting of C₁₋₈alkyl, C₁₋₈alkoxy, C₁₋₈acyl, amino, amino-C₁₋₈alkyl, halogen, hydroxy, C₁₋₈alkyl-halo, C₁₋₈alkoxy-halo, aryl, oxy-aryl, heterocyclyl, oxy-heterocyclyl, C₃₋₁₄cycloalkyl and oxy-C₃₋₁₄cycloalkyl; and
wherein C₁₋₈alkyl is optionally substituted with one, two or three substituents each selected from the group consisting of C₁₋₈alkoxy, amino, amino-C₁₋₈alkyl, halogen, hydroxy and carbonyl-C₁₋₈alkoxy.

Illustrative of the invention is a pharmaceutical composition comprising a pharmaceutically acceptable carrier and any of the compounds described above. An illustration of the invention is a pharmaceutical composition made by mixing any of the compounds described above and a pharmaceutically acceptable carrier. Illustrating the invention is a process for making a pharmaceutical composition comprising mixing any of the compounds described above and a pharmaceutically acceptable carrier.

Exemplifying the invention are methods of treating, preventing or ameliorating disorders and conditions mediated by the ORL-1 receptor in a subject in need thereof comprising administering to the subject an effective amount of any of the compounds or pharmaceutical compositions described above.

An example of the invention is a method of treating, preventing or ameliorating a condition selected from the group consisting of anxiety, depression, panic, mania, dementia, bipolar disorder, substance abuse, neuropathic pain, acute pain, chronic pain migraine, asthma, cough, psychosis, schizophrenia, epilepsy, hypertension, obesity, eating disorders, cravings, diabetes, cardiac arrhythmia, irritable bowel syndrome, Crohn's disease, urinary incontinence, adrenal disorders, attention deficit disorder, attention deficit hyperactivity disorder, Alzheimer's disease, for improved cognition or memory and for mood stabilization, in a subject in need thereof comprising administering to the subject a therapeutically effective amount of any of the compounds or pharmaceutical compositions described above.

An example of a compound of formula (I) includes a compound wherein X is CH.

An example of a compound of formula (I) includes a compound wherein X is N.

An example of a compound of formula (I) includes a compound wherein R¹ is hydrogen or one, two, three or four substituents each selected from the group consisting of C₁₋₈alkyl, C₁₋₈alkoxy, amino, halogen, hydroxy, cyano and nitro.

An example of a compound of formula (I) includes a compound wherein R¹ is hydrogen or one, two, three or four substituents each selected from the group consisting of halogen and cyano.

An example of a compound of formula (I) includes a compound wherein R² is selected from the group consisting of hydrogen, C₃₋₁₄cycloalkyl and C₁₋₈alkyl; optionally substituted on C₁₋₈alkyl with one, two or three substituents each selected from the group consisting of amino, amino-C₁₋₈alkyl, hydroxy, carbonyl-C₁₋₈alkoxy, aryl, heterocyclyl and oxy-heterocyclyl.

An example of a compound of formula (I) includes a compound wherein R³ is hydrogen.

An example of a compound of formula (I) includes a compound wherein R³ is C₁₋₈alkyl.

An example of a compound of formula (I) includes a compound wherein R⁴ is one substituent, when the double bond between position 3 and 4 in formula (I) is present, selected from the group consisting of hydrogen, hydroxy and oxy-C₁₋₈acyl.

An example of a compound of formula (I) includes a compound wherein R⁴ is two substituents, when the double bond between position 3 and 4 in formula (I) is not present, each selected from the group consisting of hydrogen, hydroxy and oxy-C₁₋₈acyl.

An example of a compound of formula (I) includes a compound wherein R⁵ is selected from the group consisting of hydrogen, C₁₋₈alkyl, carbonyl-C₁₋₈alkoxy, C₁₋₈acyl-heterocyclyl, C₃₋₁₄cycloalkyl, C₁₋₈alkyl-C₃₋₁₄cycloalkyl, carbonyl-C₃₋₁₄cycloalkyl and C₁₋₈alkyl-aryl,
wherein carbonyl-C₃₋₁₄cycloalkyl is optionally substituted on C₃₋₁₄cycloalkyl with one C₁₋₈alkyl substituent,
wherein C₁₋₈alkyl-aryl is optionally substituted on aryl with one substituent selected from the group consisting of C₁₋₈alkyl, C₁₋₈alkoxy, C₁₋₈acyl, amino, amino-C₁₋₈alkyl, halogen, hydroxy, C₁₋₈alkyl-halo, C₁₋₈alkoxy-halo, aryl, oxy-aryl, heterocyclyl, oxy-heterocyclyl, C₃₋₁₄cycloalkyl and oxy-C₃₋₁₄cycloalkyl, and
wherein C₁₋₈alkyl is optionally substituted with one substituent selected from the group consisting of C₁₋₈alkoxy, amino, amino-C₁₋₈alkyl, halogen, hydroxy and carbonyl-C₁₋₈alkoxy.

An example of a compound of formula (I) includes a compound wherein R⁵ is selected from the group consisting of hydrogen, C₁₋₈alkyl, carbonyl-C₁₋₈alkoxy, C₁₋₈acyl-heterocyclyl, C₃₋₁₄cycloalkyl, C₁₋₈alkyl-C₃₋₁₄cycloalkyl, carbonyl-C₃₋₁₄cycloalkyl and C₁₋₈alkyl-aryl,
wherein carbonyl-C₃₋₁₄cycloalkyl is optionally substituted on C₃₋₁₄cycloalkyl with one C₁₋₈alkyl substituent, and
wherein C₁₋₈alkyl-aryl is optionally substituted on aryl with one substituent selected from the group consisting of C₁₋₈alkyl and oxy-aryl.

An example of a compound of formula (I) includes a compound of formula (Ia): 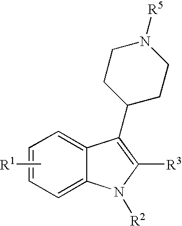
or a form thereof, wherein
R¹ is hydrogen or one, two, three or four substituents each selected from the group consisting of halogen and cyano;
R² is selected from the group consisting of hydrogen, C₃₋₁₄cycloalkyl and C₁₋₈alkyl; optionally substituted on C₁₋₈alkyl with one, two or three substituents each selected from the group consisting of amino, amino-C₁₋₈alkyl, hydroxy, carbonyl-C₁₋₈alkoxy, aryl, heterocyclyl and oxy-heterocyclyl;
R³ is selected from the group consisting of hydrogen and C₁₋₈alkyl; and
R⁵ is selected from the group consisting of hydrogen, C₁₋₈alkyl, carbonyl-C₁₋₈alkoxy, C₁₋₈acyl-heterocyclyl, C₃₋₁₄cycloalkyl, C₁₋₈alkyl-C₃₋₁₄cycloalkyl, carbonyl-C₃₋₁₄cycloalkyl and C₁₋₈alkyl-aryl,
wherein carbonyl-C₃₋₁₄cycloalkyl is optionally substituted on C₃₋₁₄cycloalkyl with one C₁₋₈alkyl substituent, and
wherein C₁₋₈alkyl-aryl is optionally substituted on aryl with one substituent selected from the group consisting of C₁₋₈alkyl and oxy-aryl.

An example of a compound of formula (Ia) is a compound wherein R¹, R², R³ and R⁵ are dependently selected from:

| Cpd | R¹ | R² | R³ | R⁵ |
|---|---|---|---|---|
| 1 | 5-F | H | H | CH₂-1-naphthyl |
| 2 | 5-F | H | H | CH₂-1-(8-CH₃)-naphthyl |
| 3 | 5-F | H | H | CH₂-cyclooctyl |
| 4 | 5-F | H | H | acenaphthen-1-yl |
| 5 | 6-Cl | benzyl | H | acenaphthen-1-yl |
| 6 | 5-Cl | H | CH₃ | acenaphthen-1-yl |
| 7 | 6-Cl | benzyl | H | CH₂-1-naphthyl |
| 8 | 6-Cl | benzyl | H | CH₂-1-(8-CH₃)-naphthyl |
| 9 | 6-Cl | benzyl | H | CH₂-cyclooctyl |
| 10 | 5-Cl | H | CH₃ | CH₂-1-naphthyl |
| 11 | 5-Cl | H | CH₃ | CH₂-1-(8-CH₃)-naphthyl |
| 12 | 5-Cl | H | CH₃ | CH₂-cyclooctyl |
| 13 | 7-Cl | H | H | CH₂-1-naphthyl |
| 14 | 7-Cl | H | H | CH₂-1-(8-CH₃)-naphthyl |
| 15 | 7-Cl | H | H | CH₂-cyclooctyl |
| 16 | 7-Cl | H | H | acenaphthen-1-yl |
| 17 | 5-F | CH₂-(1S)-oxiranyl | H | CH₂-cyclooctyl |
| 18 | 5-F | CH₂-(1S)-oxiranyl | H | CH₂-1-(8-CH₃)-naphthyl |
| 19 | 5-F | CH₂—(R)—CH(OH)—CH₂N(CH₃)₂ | H | CH₂-cyclooctyl |
| 20 | 5-F | CH₂—(R)—CH(OH)—CH₂N(CH₃)₂ | H | CH₂-1-(8-CH₃)-naphthyl |
| 21 | 6-F | H | H | C(O)OC(CH₃)₃ |
| 22 | 6-F | H | H | acenaphthen-1-yl |
| 23 | 6-F | CH₂—(R)—CH(OH)—CH₂NH₂ | H | acenaphthen-1-yl |
| 24 | 6-F | CH₂—(R)—CH(OH)—CH₂NH(CH₃) | H | acenaphthen-1-yl |
| 25 | 6-F | CH₂—(R)—CH(OH)—CH₂N(CH₃)₂ | H | acenaphthen-1-yl |
| 26 | H | H | H | H |
| 27 | 6-F | H | H | H |
| 28 | H | benzyl | H | H |
| 29 | 5-Cl | H | H | H |
| 57 | 5-Cl | H | H | benzyl |
| 58 | 7-Cl | H | H | H |

An example of a compound of formula (I) includes a compound of formula (Ib): 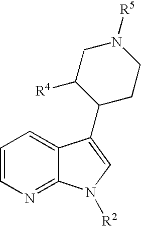
or a form thereof, wherein
R² is selected from the group consisting of hydrogen, C₃₋₁₄cycloalkyl and C₁₋₈alkyl; optionally substituted on C₁₋₈alkyl with one, two or three substituents each selected from the group consisting of amino, amino-C₁₋₈alkyl, hydroxy, carbonyl-C₁₋₈alkoxy, aryl, heterocyclyl and oxy-heterocyclyl;
R⁴ is two substituents each selected from the group consisting of hydrogen, hydroxy and oxy-C₁₋₈acyl; and
R⁵ is selected from the group consisting of hydrogen, C₁₋₈alkyl, carbonyl-C₁₋₈alkoxy, C₁₋₈acyl-heterocyclyl, C₃₋₁₄cycloalkyl, C₁₋₈alkyl-C₃₋₁₄cycloalkyl, carbonyl-C₃₋₁₄cycloalkyl and C₁₋₈alkyl-aryl,
wherein carbonyl-C₃₋₁₄cycloalkyl is optionally substituted on C₃₋₁₄cycloalkyl with one C₁₋₈alkyl substituent, and
wherein C₁₋₈alkyl-aryl is optionally substituted on aryl with one substituent selected from the group consisting of C₁₋₈alkyl and oxy-aryl.

An example of a compound of formula (Ib) is a compound wherein R², R⁴ and R⁵ are dependently selected from:

| Cpd | R² | R⁴ | R⁵ |
|---|---|---|---|
| 30 | H | H | H |
| 31 | H | H | CH₂-1-(8-CH₃)-naphthyl |
| 32 | H | H | CH₂-cyclooctyl |
| 33 | H | H | CH₂-1-naphthyl |
| 34 | H | H | acenaphthen-1-yl |
| 35 | acenaphthen-1-yl | H | acenaphthen-1-yl |
| 36 | CH₃ | H | acenaphthen-1-yl |
| 37 | (CH₂)₂-tetrahydro- | H | acenaphthen-1-yl |
| | pyran-2-yloxy | | |
| 38 | (CH₂)₂—OH | H | acenaphthen-1-yl |
| 39 | H | OC(O)CH₃ | acenaphthen-1-yl |
| 60 | H | OC(O)CH₃ | H |
| 40 | H | OH | acenaphthen-1-yl |
| 41 | C(O)OC(CH₃)₃ | OH | C(O)OC(CH₃)₃ |
| 52 | H | H | C(O)-cyclohexyl |
| 53 | H | H | hexyl |
| 54 | H | H | CH₂-cyclopropyl |
| 55 | H | H | CH₂-4-phenoxy-phenyl |
| 56 | H | H | C(O)CH₂- |
| | | | benzo[1,3]dioxol-5-yl |
| 59 | H | H | C(O)-4-C(CH₃)₃- |
| | | | cyclohexyl |

An example of a compound of formula (I) includes a compound of formula (Ic): 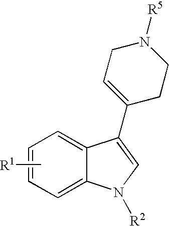
or a form thereof, wherein
R¹ is hydrogen or one, two, three or four substituents each selected from the group consisting of halogen and cyano;
R² is selected from the group consisting of hydrogen, C₃₋₁₄cycloalkyl and C₁₋₈alkyl; optionally substituted on C₁₋₈alkyl with one, two or three substituents each selected from the group consisting of amino, amino-C₁₋₈alkyl, hydroxy, carbonyl-C₁₋₈alkoxy, aryl, heterocyclyl and oxy-heterocyclyl; and
R⁵ is selected from the group consisting of hydrogen, C₁₋₈alkyl, carbonyl-C₁₋₈alkoxy, C₁₋₈acyl-heterocyclyl, C₃₋₁₄cycloalkyl, C₁₋₈alkyl-C₃₋₁₄cycloalkyl, carbonyl-C₃₋₁₄cycloalkyl and C₁₋₈alkyl-aryl,
wherein carbonyl-C₃₋₁₄cycloalkyl is optionally substituted on C₃₋₁₄cycloalkyl with one C₁₋₈alkyl substituent, and
wherein C₁₋₈alkyl-aryl is optionally substituted on aryl with one substituent selected from the group consisting of C₁₋₈alkyl and oxy-aryl.

An example of a compound of formula (Ic) is a compound wherein R¹, R² and R⁵ are dependently selected from:

| Cpd | R¹ | R² | R⁵ |
|---|---|---|---|
| 42 | 5-CN | H | acenaphthen-1-yl |
| 43 | 5-CN | CH₂-(1S)-oxiranyl | acenaphthen-1-yl |
| 44 | 5-CN | CH₂—(R)—CH(OH)CH₂NH₂ | acenaphthen-1-yl |
| 45 | 5-CN | CH₂—(R)—CH(OH)CH₂NH(CH₃) | acenaphthen-1-yl |
| 46 | 5-CN | CH₂—(R)—CH(OH)CH₂N(CH₃)₂ | acenaphthen-1-yl |
| 47 | 6-F | H | C(O)OC(CH₃)₃ |
| 48 | 6-F | H | H |
| 49 | 6-F | H | acenaphthen-1-yl |
| 50 | 6-F | CH₂CH₃ | acenaphthen-1-yl |
| 51 | 5-Cl | H | benzyl |

An example of a compound of formula (I) includes a compound selected from: 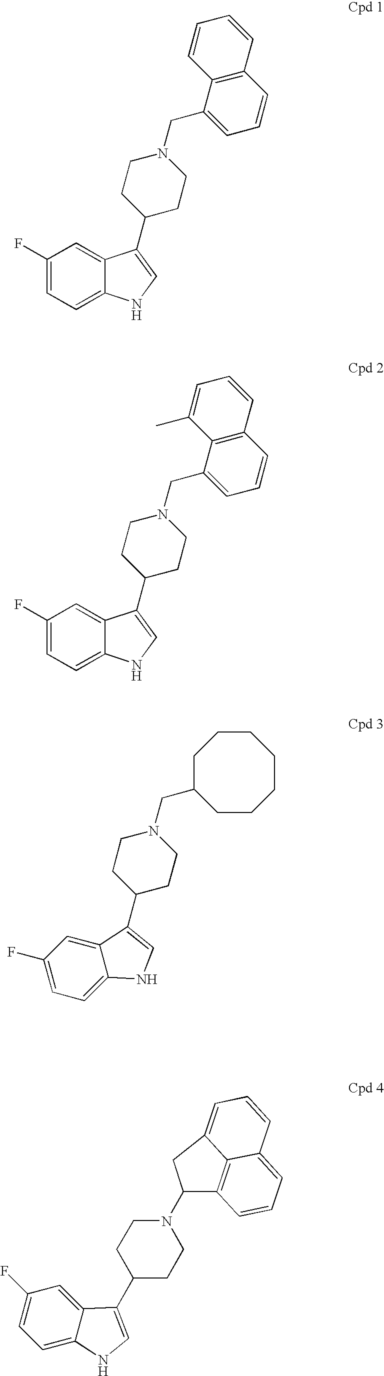 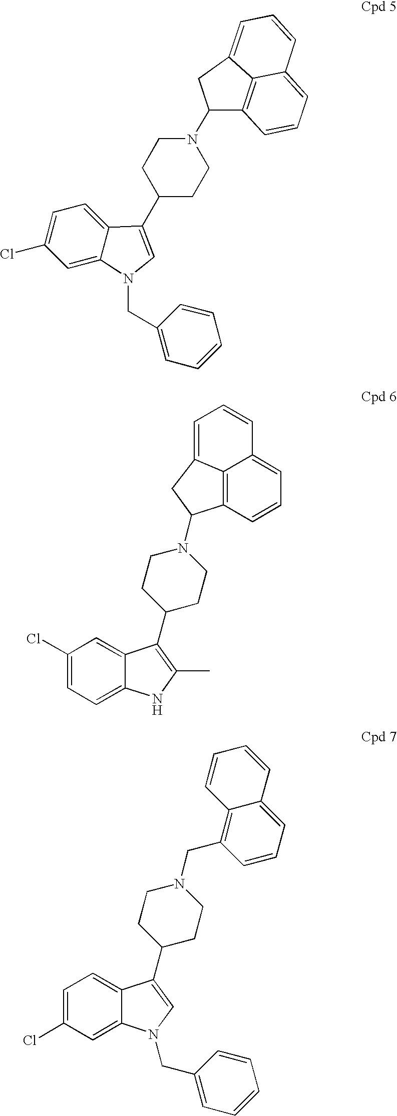 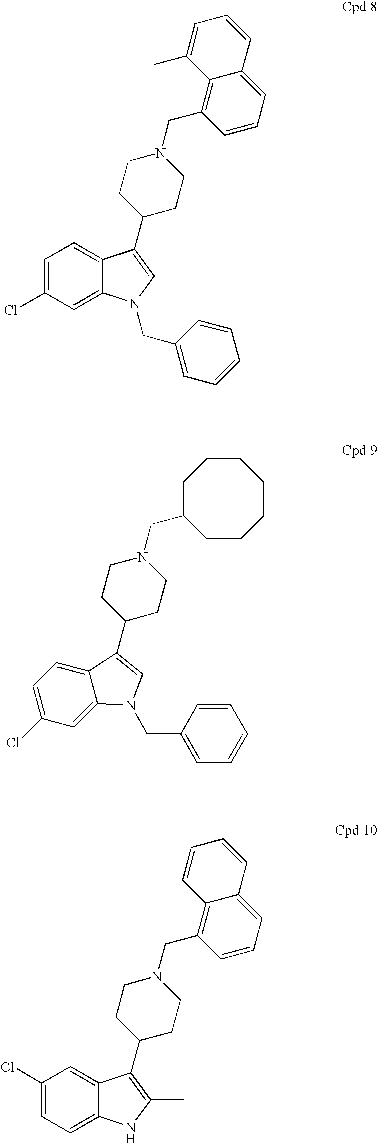 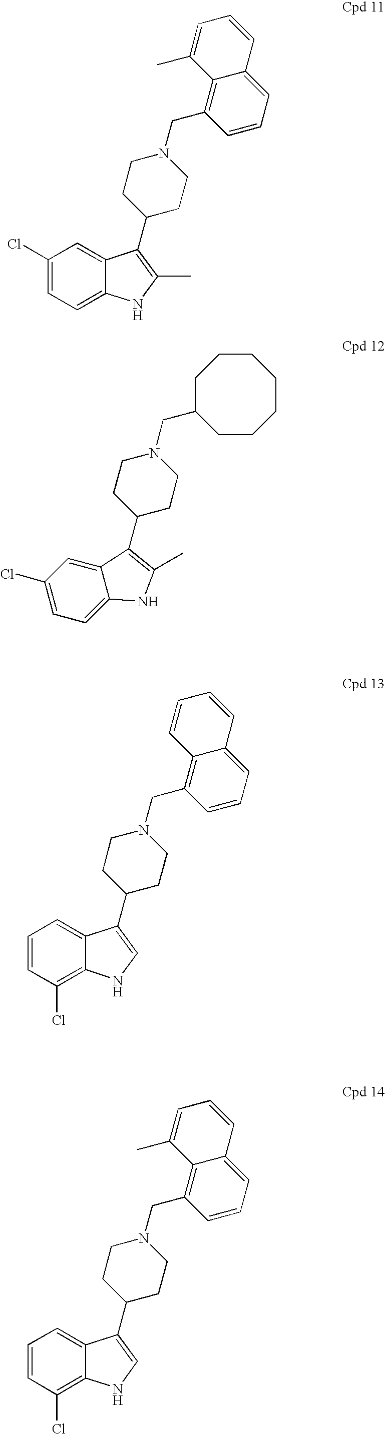 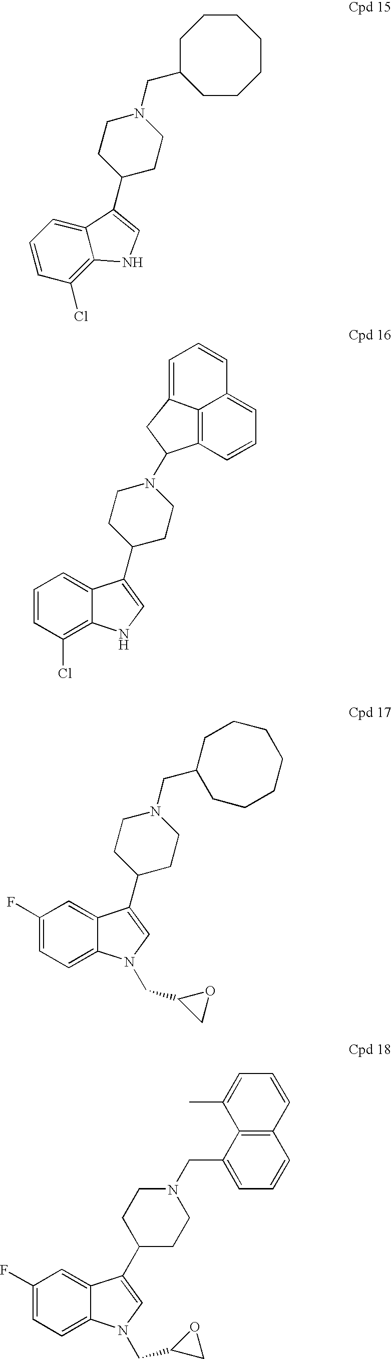 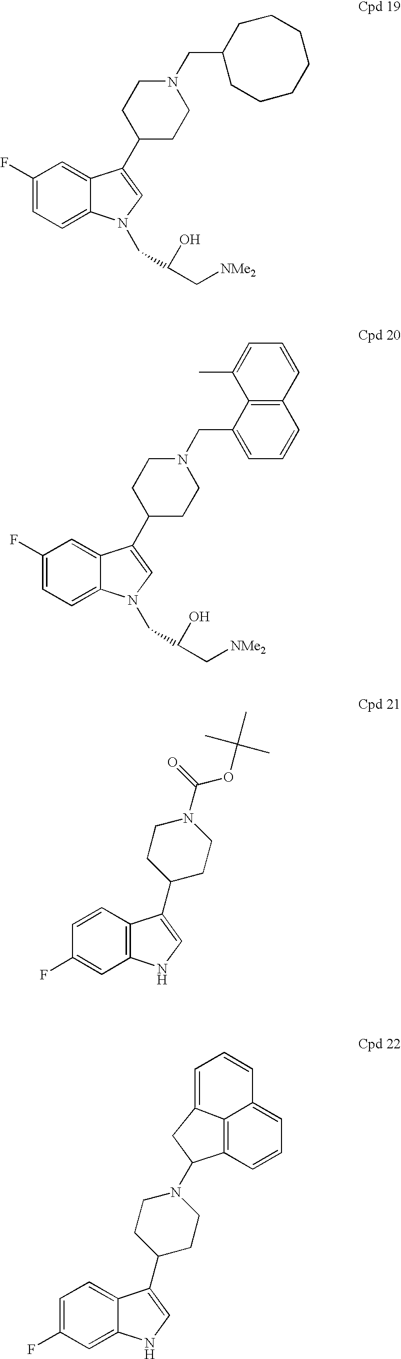 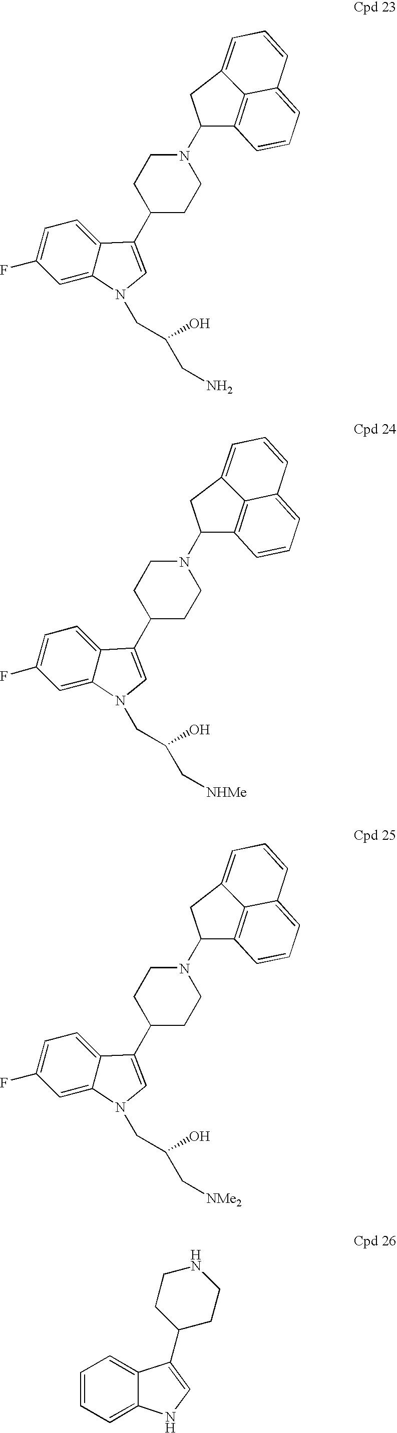 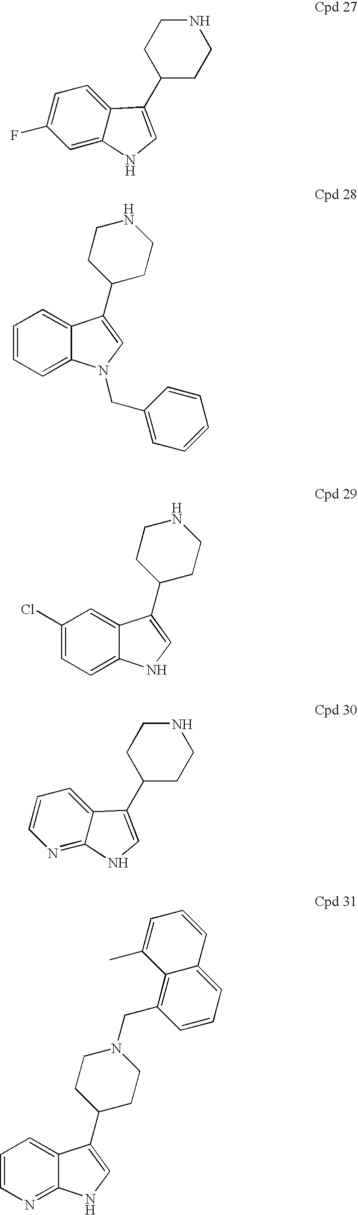 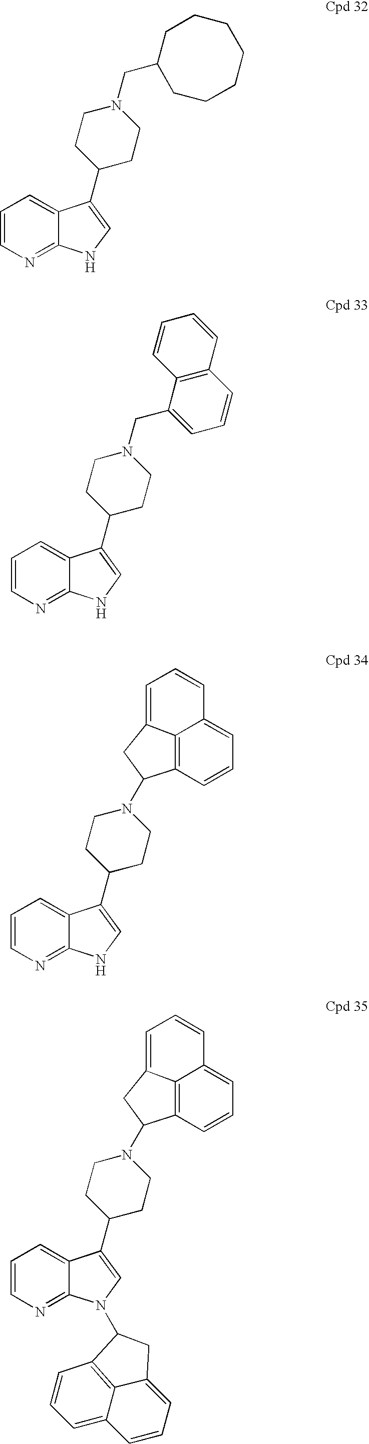 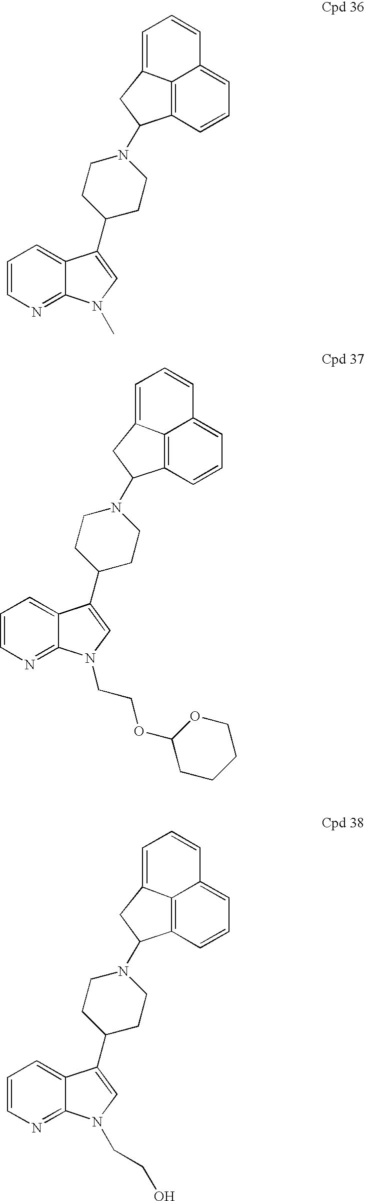 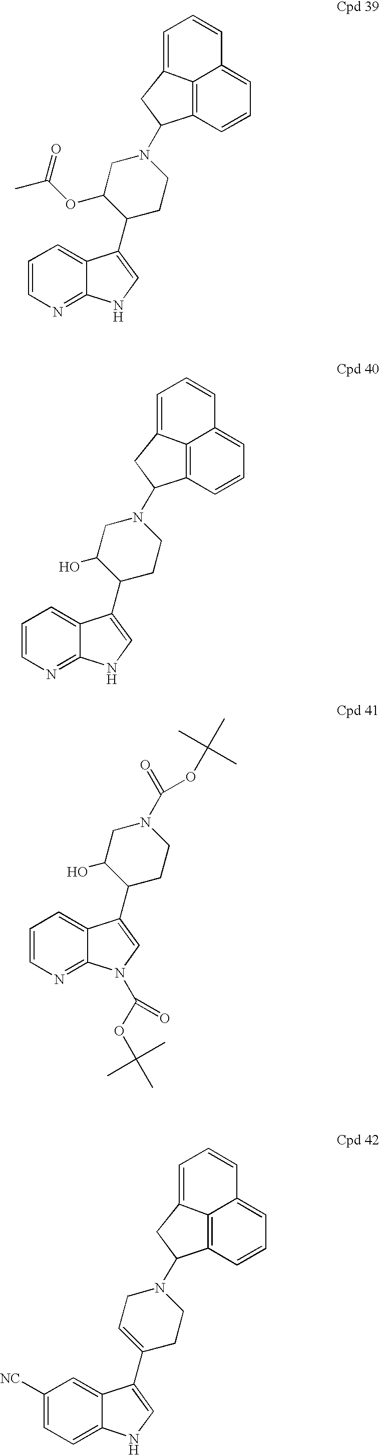 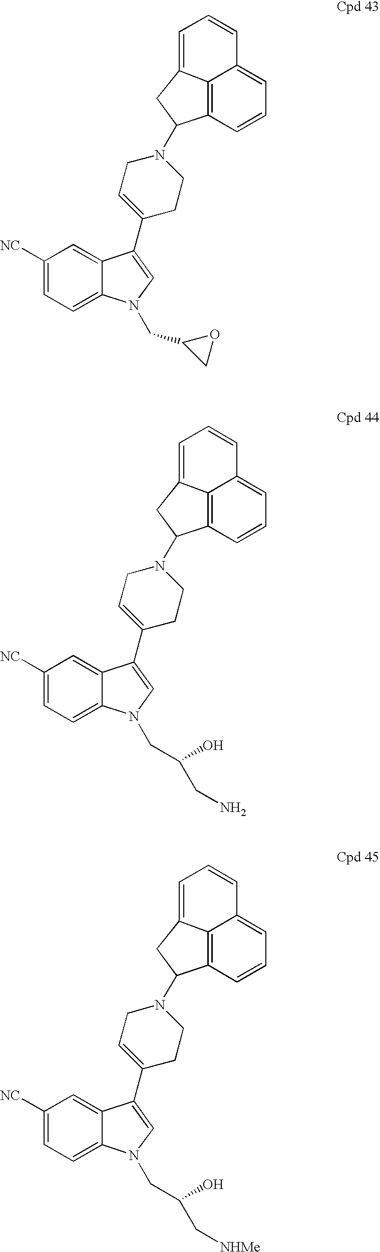 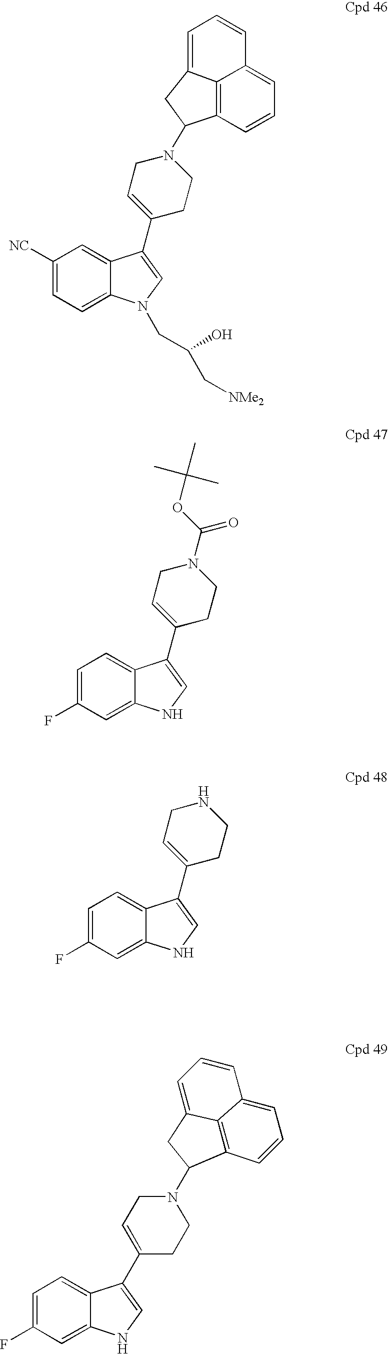 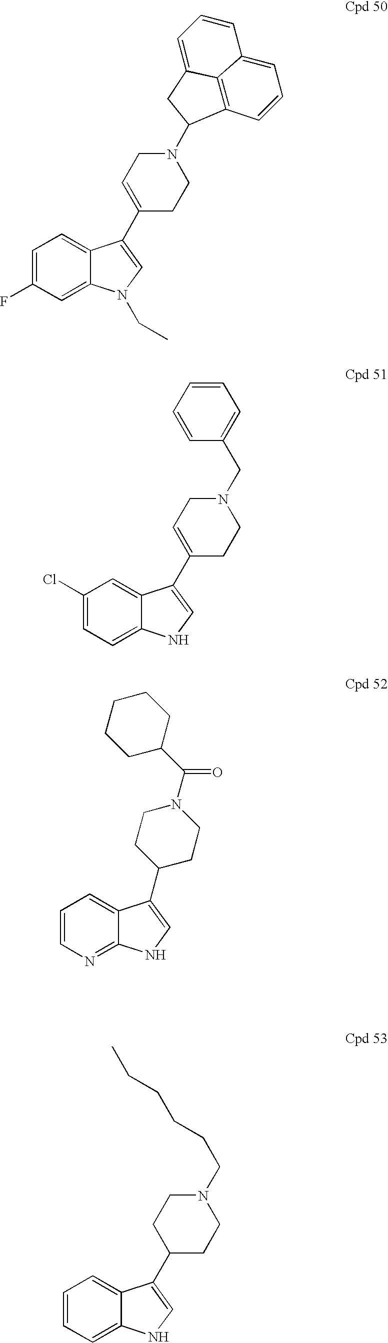 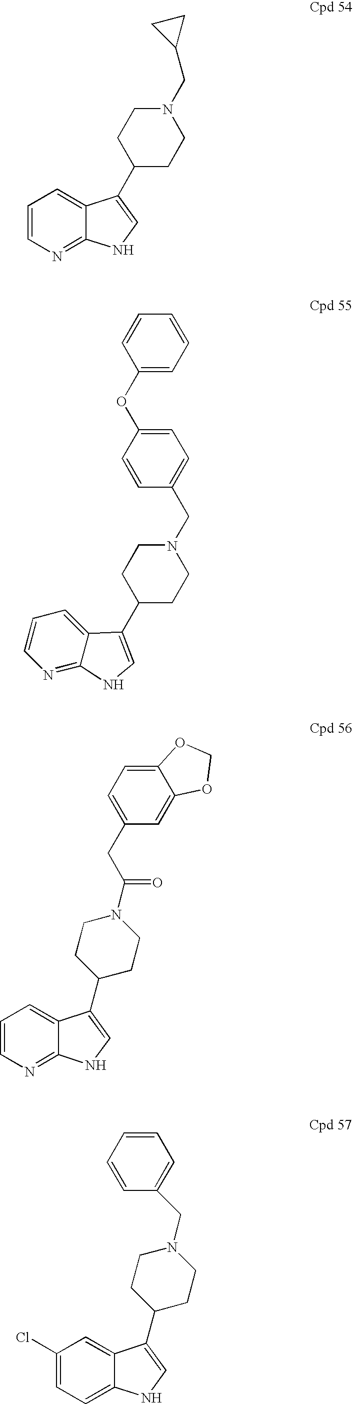 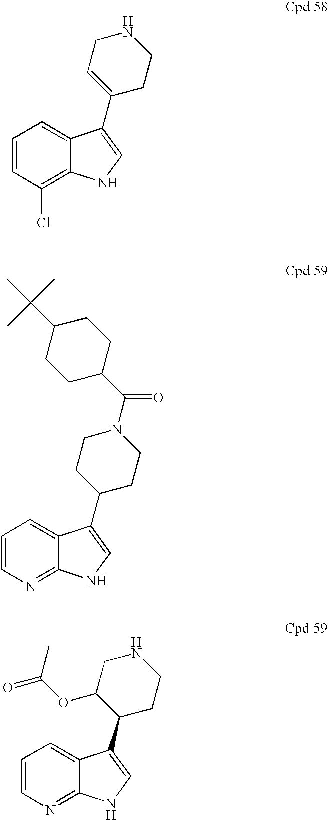
Chemical Definitions

As used herein, the following terms have the following meanings:

The term “C₁₋₈alkyl,” whether used alone or as part of a substituent group, means a saturated branched or straight chain monovalent hydrocarbon radical or alkyldiyl linking group having a specified number of carbon atoms, wherein the radical is derived by the removal of one hydrogen atom from a single carbon atom and the alkyldiyl linking group is derived by the removal of one hydrogen atom from each of two carbon atoms in the chain. The term “C₁₋₈alkyl” refers to a radical having from 1-8 carbon atoms in a linear or branched arrangement. Typical alkyl radicals include, but are not limited to, methyl, ethyl, 1-propyl, 2-propyl, 1-butyl, 2-butyl, tert-butyl, 1-pentyl, 2-pentyl, 3-pentyl, 1-hexyl, 2-hexyl, 3-hexyl, 1-heptyl, 2-heptyl, 3-heptyl, 1-octyl, 2-octyl, 3-octyl and the like. Embodiments include, e.g., the alkyl groups C₁₋₈alkyl or C₁₋₄alkyl. Alkyl and alkyldiyl radicals may be attached to a core molecule via a terminal carbon atom or via a carbon atom within the chain. Similarly, any number of substituent variables may be attached to an alkyl or alkyldiyl radical when allowed by available valences.

The term “C₁₋₈alkoxy,” whether used alone or as part of a substituent group, means an alkyl or alkyldiyl alcohol radical derived by the removal of the hydrogen atom from the hydroxide oxygen portion of the alcohol radical. Typical embodiments include, e.g., the alkoxy groups C₁₋₈alkoxy or C₁₋₄alkoxy. For example, “C₁₋₈alkoxy” specifically includes the radicals methoxy, ethoxy, propoxy, butoxy, pentoxy, hexoxy, heptoxy, octoxy and the like. As described above, an alkoxy radical may be similarly attached to a core molecule and further substituted where indicated.

The term “C₃₋₁₄cycloalkyl,” whether used alone or as part of a substituent group, means a saturated or partially unsaturated cyclic hydrocarbon ring system. Examples include C₃₋₈cycloalkyl, C₅₋₈cycloalkyl, C₅₋₁₂cycloalkyl, C₉₋₁₃cycloalkyl and the like. Typical cycloalkyl radicals include, but are not limited to, cyclopropyl, cyclobutyl, cyclopentyl, cyclohexyl, indanyl, fluorenyl, acenaphthenyl and the like.

The term “heterocyclyl,” whether used alone or as part of a substituent group, means a saturated, partially unsaturated or completely unsaturated cyclic ring radical derived by the removal of one hydrogen atom from a single carbon atom of the ring system and in which one or more ring carbon atoms are a heteroatom selected from N, O, S, SO or SO₂. Embodiments include rings wherein 1, 2, 3 or 4 members of the ring are a nitrogen atom, or 0, 1, 2 or 3 members of the ring are nitrogen atoms and 1 member is an oxygen or sulfur atom.

Typical saturated or partially unsaturated heterocyclyl radicals include, and are not limited to, oxiranyl, dihydro-1H-pyrrole (including 2-pyrrolinyl or 3-pyrrolinyl), pyrrolidinyl, 1,3-dioxolanyl, 2-imidazolinyl (also referred to as 4,5-dihydro-1H-imidazolyl), imidazolidinyl, 2-pyrazolinyl, pyrazolidinyl, tetrazolyl, pyran, tetrahydropyranyl, tetrahydrothiopyranyl, piperidinyl, 1,4-dioxanyl, morpholinyl, 1,4-dithianyl, thiomorpholinyl, piperazinyl, azetidinyl, azepanyl, hexahydro-1,4-diazepinyl, hexahydro-1,4-oxazepanyl, tetrahydro-furyl, tetrahydro-thienyl, tetrahydro-pyranyl, tetrahydro-pyridazinyl, 1,3-benzodioxol-5-yl (also referred to as benzo [1,3]dioxol-5-yl), 2,3-dihydro-1,4-benzodioxin-6-yl and the like.

Typical completely unsaturated heterocyclyl radicals include, and are not limited to, furyl, thienyl, pyrrolyl, oxazolyl, thiazolyl, imidazolyl, pyrazolyl, isoxazolyl, isothiazolyl, oxadiazolyl, triazolyl, tetrazolyl, thiadiazolyl, pyridinyl, pyridazinyl, pyrimidinyl, pyrazinyl, indolizinyl, indolyl, isoindolyl, benzo[b]furyl, benzo[b]thienyl, indazolyl, benzimidazolyl, benzoxazolyl, benzthiazolyl, purinyl, 4H-quinolizinyl, quinolinyl, isoquinolinyl, cinnolinyl, phthalzinyl, quinazolinyl, quinoxalinyl, 1,8-naphthyridinyl, pteridinyl and the like.

The term “aryl,” whether used alone or as part of a substituent group, means a completely unsaturated cyclic ring radical derived by the removal of one hydrogen atom from a single carbon atom of the ring system. Typical aryl radicals include, and are not limited to, phenyl, naphthalenyl, indenyl, azulenyl, anthracenyl, biphenyl and the like.

As used herein, C₁₋₈acyl means a radical of the formula: —C(O)—C₁₋₈alkyl.

As used herein, amino means a radical of the formula: —NH₂.

As used herein, amino-C₁₋₈alkyl means a radical of the formula: —NH—C₁₋₈alkyl or N(C₁₋₈alkyl)₂.

As used herein, carbonyl means a linking group of the formula: —C(O)—.

As used herein, carbonyl-C₁₋₈alkoxy means a radical of the formula: —C(O)—O—C₁₋₈alkyl.

As used herein, oxy-aryl, oxy-heterocyclyl and oxy-C₃₋₈cycloalkyl mean radicals of the formula, respectively: —O-aryl, —O-heterocyclyl or —O—C₃₋₈cycloalkyl.

As used herein, halogen or halo means the group chloro, bromo, fluoro or iodo.

As used herein, C₁₋₈alkyl-halo means a radical of the formula: —C₁₋₈alkyl(halo)₁₋₃ and includes monofluoromethyl, difluoromethyl, trifluoromethyl, trifluoroethyl and the like.

As used herein, C₁₋₈alkoxy-halo means a radical of the formula: —C₁₋₈alkoxy(halo)₁₋₃ and includes monofluoromethoxy, difluoromethoxy, trifluoromethoxy, trifluoroethoxy and the like.

As used herein, oxy-C₁₋₈acyl means a radical of the formula: —OC(O)—C₁₋₈alkyl.

The term “substituted” means the independent replacement of one or more hydrogen atoms within a radical with that amount of substitutents allowed by available valences.

The term “dependently selected” means that the structure variables are specified in an indicated combination.

In general, IUPAC nomenclature rules are used throughout this disclosure.

Compound Forms

The term “forms” and “forms thereof” means that the compounds of the present invention may exist in various salt, stereoisomer, crystalline, solvate, ester, prodrug or active metabolite forms. The present invention encompasses all such compound forms, including active compounds in the form of essentially pure enantiomers, racemic mixtures and tautomers.

The compounds of the invention may be present in the form of pharmaceutically acceptable salts. For use in medicines, the “pharmaceutically acceptable salts” of the compounds of this invention refer to non-toxic acidic/anionic or basic/cationic salt forms.

Pharmaceutically acceptable acidic/anionic salts include the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate and triethiodide salts.

Organic or inorganic acids also include, and are not limited to, hydriodic, perchloric, sulfuric, phosphoric, propionic, glycolic, methanesulfonic, hydroxyethanesulfonic, oxalic, 2-naphthalenesulfonic, p-toluenesulfonic, cyclohexanesulfamic, saccharinic or trifluoroacetic acid.

Pharmaceutically acceptable basic/cationic salts include, and are not limited to aluminum, 2-amino-2-hydroxymethyl-propane-1,3-diol, ammonia, benzathine, t-butylamine, calcium, calcium gluconate, calcium hydroxide, chloroprocaine, choline, choline bicarbonate, choline chloride, cyclohexylamine, diethanolamine, ethylenediamine, lithium, LiOMe, L-lysine, magnesium, meglumine, NH₃, NH₄OH, N-methyl-D-glucamine, piperidine, potassium, potassium-t-butoxide, potassium hydroxide (aqueous), procaine, quinine, sodium, sodium carbonate, sodium-2-ethylhexanoate, sodium hydroxide, triethanolamine or zinc.

During any of the processes for preparation of the compounds of the invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, 3^{rd} Edition, John Wiley & Sons, 1999. The protecting groups may be removed at a convenient subsequent stage using methods known in the art.

The invention includes compounds of various isomers and mixtures thereof. The term “isomer” refers to compounds that have the same composition and molecular weight but differ in physical and/or chemical properties. Such substances have the same number and kind of atoms but differ in structure. The structural difference may be in constitution (geometric isomers) or in an ability to rotate the plane of polarized light (stereoisomers).

The term “stereoisomer” means isomers of identical constitution that differ in the spatial arrangement of their atoms. Enantiomers and diastereomers are stereoisomers wherein an asymmetrically substituted carbon atom acts as a chiral center.

The term “chiral” means a molecule that is not superimposable on its mirror image, implying the absence of an axis and a plane or center of symmetry. The term “enantiomer” means one of a pair of molecular species that are mirror images of each other and are not superimposable. The term “diastereomer” means stereoisomers that are not related as mirror images. The symbols “R” and “S” represent the configuration of substituents around a chiral carbon atom(s).

The term “racemate” or “racemic mixture” means a compound of equimolar quantities of two enantiomeric species, wherein the compound is devoid of optical activity. The term “optical activity” means the degree to which a chiral molecule or non-racemic mixture of chiral molecules rotates the plane of polarized light.

The term “geometric isomer” means isomers that differ in the orientation of substituent atoms in relationship to a carbon-carbon double bond, to a cycloalkyl ring, or to a bridged bicyclic system. Substituent atoms (other than H) on each side of a carbon-carbon double bond may be in an E or Z configuration. In the “E” configuration, the substituents are on opposite sides in relationship to the carbon-carbon double bond. In the “Z” configuration, the substituents are oriented on the same side in relationship to the carbon-carbon double bond.

The isomeric descriptors (“R,” “S,” “E,” and “Z”) indicate atom configurations relative to a core molecule and are intended to be used as defined in the literature.

The compounds of the invention may be prepared as individual isomers by either isomer-specific synthesis or resolved from an isomeric mixture. Conventional resolution techniques include combining the free base (or free acid) of each isomer of an isomeric pair using an optically active acid (or base) to form an optically active salt (followed by fractional crystallization and regeneration of the free base), forming an ester or amide of each of the isomers of an isomeric pair by reaction with an appropriate chiral auxiliary (followed by fractional crystallization or chromatographic separation and removal of the chiral auxiliary), or separating an isomeric mixture of either an intermediate or a final product using various well known chromatographic methods.

Furthermore, compounds of the invention may have one or more polymorph or amorphous crystalline forms. Said forms are included in the scope of the invention. In addition, some of the compounds may form solvates with water (i.e., hydrates) or common organic solvents. Said solvates are encompassed within the scope of this invention.

Therapeutic Use

The compounds of formula (I) are modulators of the ORL-1 receptor, having an IC₅₀ (50% inhibition concentration) or an EC₅₀ (50% effective concentration) in a range of about 50 μM or less, of about 25 μM or less, of about 15 μM or less, of about 10 μM or less, of about 5 μM or less, of about 1 μM or less, of about 0.5 μM or less, of about 0.25 μM or less or of about 0.1 μM or less.

Accordingly, the modulator compounds of the present invention are useful as agonists, inverse agonists or antagonists of the ORL-1 receptor for treating, preventing or ameliorating an ORL-1 receptor mediated disorder or condition.

The present invention includes a method for use of the compounds of formula (I) for mediating ORL-1 receptor activity comprising contacting the receptor with one or more of the compounds.

The present invention is further directed to a method for treating, preventing or ameliorating ORL-1 receptor mediated disorders and conditions in a subject in need thereof comprising administering to the subject an effective amount of one or more compounds of formula (I) or a pharmaceutical composition thereof.

An example of the method includes administering to the subject an effective amount of a compound of formula (I) or composition thereof in the form of a medicament. Consequently, the invention encompasses the use of the compound of formula (I) as a medicament.

An example of the method includes use of a compound of formula (I) as a marker, wherein the compound is labeled with a ligand such as a radioligand (selected from deuterium, tritium and the like).

The present invention includes the use of a compound of formula (I) for the manufacture of a medicament for treating, preventing or ameliorating any of the disorders or conditions mentioned in any of the foregoing methods.

The term “treating, preventing or ameliorating” includes, and is not limited to, facilitating the eradication of, inhibiting the progression of or promoting stasis of an ORL-1 receptor mediated disorder or condition.

The foregoing methods contemplate that the compounds of the present invention are therapeutically useful for treating, preventing or ameliorating ORL-1 receptor mediated disorders and conditions such as, without limitation, anxiety, depression, panic, dementia, mania, bipolar disorder, substance abuse, neuropathic pain, acute pain, chronic pain, migraine, asthma, cough, psychosis, schizophrenia, epilepsy, hypertension, obesity, eating disorders, cravings, diabetes, cardiac arrhythmia, irritable bowel syndrome, Crohn's disease, urinary incontinence, adrenal disorders, attention deficit disorder, attention deficit hyperactivity disorders or Alzheimer's disease and are further useful for improved cognition or memory and mood stabilization.

The term “administering,” with respect to the methods of the present invention, refers to a means for treating, ameliorating or preventing a disorder or condition as described herein with a compound specifically disclosed or a compound or prodrug thereof, which would obviously be included within the scope of the invention albeit not specifically disclosed for certain of the instant compounds.

Such methods include prophylactically or therapeutically administering an effective amount of one or more compounds of formula (I) or a composition or medicament thereof at different times during the course of a therapy or concurrently in a combination form. Prophylactic administration can occur prior to the manifestation of symptoms characteristic of an ORL-1 receptor mediated disorder or condition such that the disease or disorder is prevented or, alternatively, delayed in its progression. The instant invention is therefore to be understood as embracing all such regimes of simultaneous or alternating treatment and the term “administering” is to be interpreted accordingly.

The term “prodrug” refers to a metabolic precursor of a compound of formula (I) or pharmaceutically acceptable form thereof. In general, a prodrug is a functional derivative of a compound which may be inactive when administered to a subject but is readily convertible in vivo into an active metabolite compound.

The term “active metabolite” refers to a metabolic product of a compound that is pharmaceutically acceptable and effective. Conventional procedures for the selection and preparation of suitable prodrug derivatives are described, for example, in “Design of Prodrugs”, ed. H. Bundgaard, Elsevier, 1985.

The term “subject” as used herein, refers to a patient, such as an animal, preferably a mammal, most preferably a human, who has been the object of treatment, observation or experiment and is at risk of (or susceptible to) developing an ORL-1 receptor mediated disorder or condition or a disorder or condition related to ORL-1 receptor mediated activity.

The term “effective amount” refers to that amount of compound or composition that elicits the biological or medicinal response (such as modulationg the activity of the ORL-1 receptor) in a tissue system, animal or human, that is being sought by a researcher, veterinarian, medical doctor, or other clinician, which includes treating, preventing or ameliorating the symptoms of the disorder or condition being treated.

The effective amount of a compound of formula (I) exemplified in such a method is from about 0.001 mg/kg/day to about 300 mg/kg/day.

The term “composition” refers to a product containing a compound of the present invention such as a product comprising the specified ingredients in the specified amounts, as well as any product which results, directly or indirectly, from such combinations of the specified ingredients in the specified amounts.

The term “medicament” refers to a product for use in treating, preventing or ameliorating an ORL-1 receptor mediated disorder or condition.

The term “pharmaceutically acceptable” refers to molecular entities and compositions that are of sufficient purity and quality for use in the formulation of a composition or medicament of the present invention and that, when appropriately administered to an animal or a human, do not produce an adverse, allergic or other untoward reaction. Since both human use (clinical and over-the-counter) and veterinary use are equally included within the scope of the present invention, a pharmaceutically acceptable formulation would include a composition or medicament for either human or veterinary use.

Pharmaceutical Compositions

An example of the present invention includes a pharmaceutical composition comprising an admixture of one or more compounds of formula (I) and/or one or more pharmaceutically acceptable forms thereof and one or more pharmaceutically acceptable excipients.

The pharmaceutically acceptable forms for a compound of formula (I) include a pharmaceutically acceptable salt, ester, prodrug or active metabolite of a compound of formula (I).

Pharmaceutical compositions according to the invention may, alternatively or in addition to a compound of formula I, comprise as an active ingredient a pharmaceutically acceptable salt of a compound of formula I or a prodrug or pharmaceutically active metabolite of such a compound or salt.

The present invention further includes the use of a process for making a composition or medicament comprising mixing one or more of the instant compounds and an optional pharmaceutically acceptable carrier; and, includes those compositions or medicaments resulting from such a process. Contemplated processes include both conventional and unconventional pharmaceutical techniques.

The composition or medicament may take a wide variety of forms to effectuate mode of administration, including, but not limited to, intravenous (both bolus and infusion), oral, nasal, transdermal, topical with or without occlusion, and injection intraperitoneally, subcutaneously, intramuscularly, intratumorally or parenterally.

The composition or medicament may be in a dosage unit such as a tablet, pill, capsule, powder, granule, sterile parenteral solution or suspension, metered aerosol or liquid spray, drop, ampoule, auto-injector device or suppository; for administration orally, parenterally, intranasally, sublingually or rectally or by inhalation or insufflation.

Compositions or medicaments suitable for oral administration include solid forms such as pills, tablets, caplets, capsules (each including immediate release, timed release and sustained release formulations), granules and powders; and, liquid forms such as solutions, syrups, elixirs, emulsions and suspensions.

Forms useful for parenteral administration include sterile solutions, emulsions and suspensions. Furthermore, compositions or medicaments can be administered in intranasal form via topical use of suitable intranasal vehicles, or via transdermal routes, using, e.g., those forms of transdermal skin patches well known to those of ordinary skill in that art.

Advantageously, a compound of formula (I) may be administered in a single daily dose, or the total daily dosage may be administered in divided doses of two, three or four times daily. Alternatively, the composition or medicament may be presented in a form suitable for once-weekly or once-monthly administration; for example, an insoluble salt of the active compound, such as the decanoate salt, may be adapted to provide a depot preparation for intramuscular injection.

The dosage form (tablet, capsule, powder, injection, suppository, teaspoonful and the like) containing the composition or medicament contains an effective amount of the active ingredient necessary to be therapeutically or prophylactically effective as described above.

The composition or medicament may contain from about 0.001 mg to about 5000 mg (preferably, from about 0.001 to about 500 mg) of the active compound or prodrug thereof and may be constituted into any form suitable for the mode of administration selected for a subject in need. A contemplated effective amount may range from about 0.001 mg to about 300 mg/kg of body weight per day. Preferably, the range is from about 0.003 to about 100 mg/kg of body weight per day. Most preferably, the range is from about 0.005 to about 15 mg/kg of body weight per day. The composition or medicament may be administered according to a dosage regimen of from about 1 to about 5 times per day.

For oral administration, the composition or medicament is preferably in the form of a tablet or capsule containing, e.g., 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the patient to be treated.

Optimal dosages will vary depending on factors associated with the particular patient being treated (e.g., age, weight, diet and time of administration), the severity of the condition being treated, the compound being employed, the mode of administration and the strength of the preparation. The use of either daily administration or post-periodic dosing may be employed.

Synthetic Methods

Representative compounds of the present invention can be synthesized in accordance with the general synthetic schemes described below and are illustrated more particularly in the specific synthetic examples that follow. The general schemes and specific examples are offered by way of illustration; the invention should not be construed as being limited by the chemical reactions and conditions expressed.

Except where indicated, starting materials and intermediates used in the schemes and examples are prepared by known methodologies well within the ordinary skill of persons versed in the art. No attempt has been made to optimize the yields obtained in any of the example reactions. One skilled in the art would also know how to increase such yields through routine variations in materials, solvents, reagents, reaction conditions and the like.

One skilled in the art will recognize that a variety of solvents or mixtures thereof may be used for a given reaction step and that the disclosure of a particular solvent or solvent system is not intended to act as a limitation to the scope of possible routine variations to a process for making a compound of the present invention.

When a synthesis product is described in any specific synthetic example as a “residue,” the use of the term. It will be understood by one of ordinary skill in the art that the term “residue” is also not intended as a limitation to a description of the physical state in which a reaction product is isolated and may include, for example, a solid, an oil, a foam, a gum, a syrup and the like.

The terms used in describing the invention are commonly used and known to those skilled in the art. When used herein, the following abbreviations have the indicated meanings:

| Abbreviation | Meaning |
|---|---|
| BOC or Boc | tert-butoxy-carbonyl- |
| CBz | benzyloxycarbonyl(C₆H₅—CH₂—O—C(O)—) |
| DCC | N,N-dicyclohexylcarbodiimide |
| DCM | dichloromethane |
| DIPEA or DIEA | diisopropylethylamine |
| DMF | N,N-dimethylformamide |
| DMSO | dimethylsulfoxide |
| EGTA | ethylene glycol-O,O′-bis(2-aminoethyl)-N,N,N′,N′- |
| | tetracacetic acid |
| Fmoc | 9-fluorenylmethoxycarbonyl |
| HATU | O-(7-azabenzotriazol-1-yl)-N,N,N″,N″- |
| | tetramethyluronium hexafluorophosphate |
| HOBT | 1-hydroxybenzotriazole hydrate |
| KO-t-Bu | potassium tert-butoxide |
| LiHMDS | lithium bis(trimethylsilyl)amide |
| NaHMDS | sodium bis(trimethylsilyl)amide |
| NaBH(OAc)₃ | sodium tricaetoxyborohydride |
| NAH | sodium aluminum hydride |
| NMP | N-methyl-2-pyrrolidinone |
| MeONa | sodium methoxide |
| TEA | triethylamine |
| TFA | trifluoroacetic acid |
| THF | tetrahydrofuran |
| Tris-HCl or Tris-Cl | tris[hydroxymethyl]aminomethyl hydrochloride |

Compounds representative of formula (I) may be prepared according to the process of Scheme A.

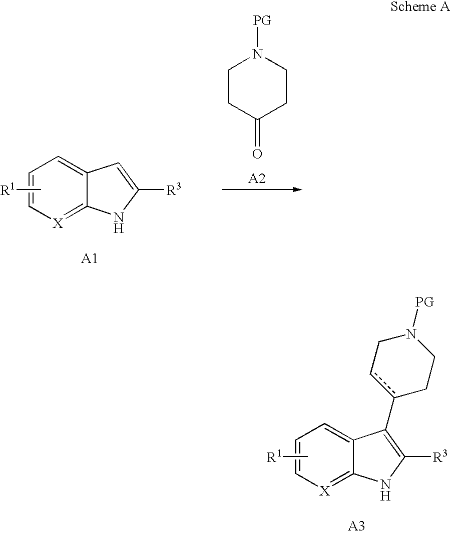

Compound A1 is reacted with a solution of Compound A2 (wherein PG is a suitable nitrogen protecting group such as Boc, CBz, Fmoc, benzhydryl, triphenylmethyl, 4-methoxybenzyl, benzoyl and the like in an organic solvent such as MeOH, EtOH, THF, NMP, DMF and the like) in the presence of a base (such as KOH, NaOH and the like), wherein the base is present in an amount equal to or greater than 10 about one molar equivalent of Compound A2, to yield a Compound A3.

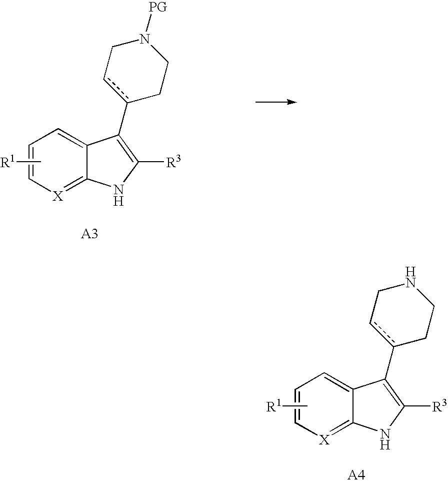

Compound A3 may be deprotected using techniques known to those skilled in the art to yield a Compound A4. For example, when PG is Boc, Compound A3 is reacted with an acid (such as TFA, HCl and the like).

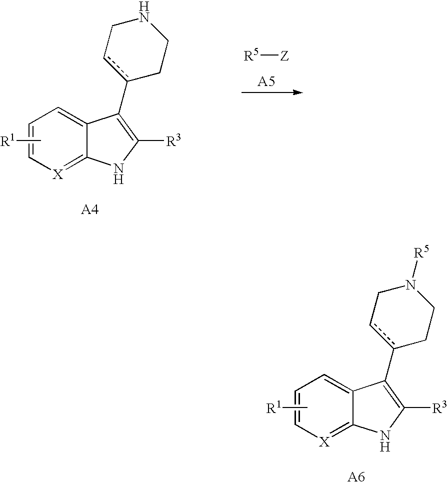

Compound A4 is reacted with a solution of Compound A5 (wherein Z is a suitable leaving group such as Cl, Br, I, tosylate, mesylate and the like in an organic solvent such as DMF, DMSO, NMP and the like) in the presence of a base (such as TEA, DIPEA, pyridine, Na₂CO₃, K₂CO₃ and the like), wherein the base is present in an amount equal to or greater than about one molar equivalent of Compound A5, to yield a Compound A6.

For example, when R⁵-Z is an aldehyde or a ketone, Compound A4 is reacted with a solution of Compound A5 in the presence of a reducing agent (such as NaBH(OAc)₃, Na(BH₃)CN and the like) to yield Compound A6.

For example, when R⁵-Z is a carboxylic acid or acid chloride, Compound A4 is reacted with a solution of Compound A5 in the presence of a coupling agent (such as HATU, DCC and the like) to yield Compound A6.

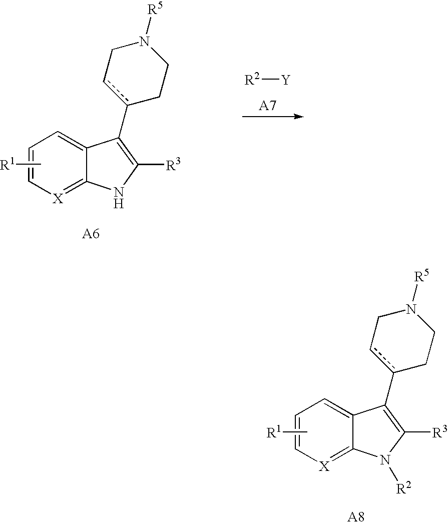

Compound A6 is reacted with a solution of Compound A7 (wherein Y is a suitable leaving group such as Cl, Br, I, tosylate, mesylate and the like in an organic solvent such as THF, NMP, DMF and the like) in the presence of a base (such as TEA, DIPEA, pyridine, Na₂CO₃, K₂CO₃ and the like), wherein the base is present in an amount equal to or greater than about one molar equivalent of Compound A7, to yield a Compound A8.

Compounds representative of formula (I) may be prepared according to the process of Scheme B.

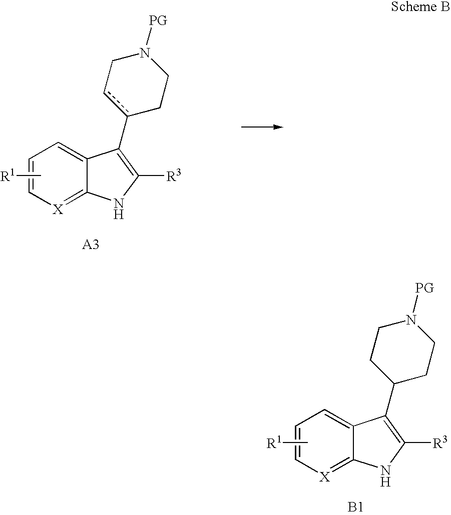

A solution of Compound A3 (in an organic solvent such as MeOH, EtOH and the like) is reacted with a catalyst (such as Pd/C, PtO₂ and the like) in the presence of hydrogen blanket having a pressure in the range of from about 1 psi to about 60 psi at a temperature in the range of from about 20° C. to about 60° C. to yield a Compound B1.

Using the procedure of Scheme A, Compound B1 is deprotected and reacted with a solution of Compound A5 in the presence of a base, wherein the base is present in an amount equal to or greater than about one molar equivalent of Compound A5, to yield an R⁵ substituted Compound B2.

Using the procedure of Scheme A, Compound B2 is reacted with a solution of Compound A7 in the presence of a base (such as NaH, KH, sodium trimethylsilylamide, NaHMDS, LiHMDS and the like), wherein the base is present in an amount equal to or greater than about one molar equivalent of Compound A7, to yield an R² substituted Compound B3.

Compounds representative of formula (I) may be prepared according to the process of Scheme C.

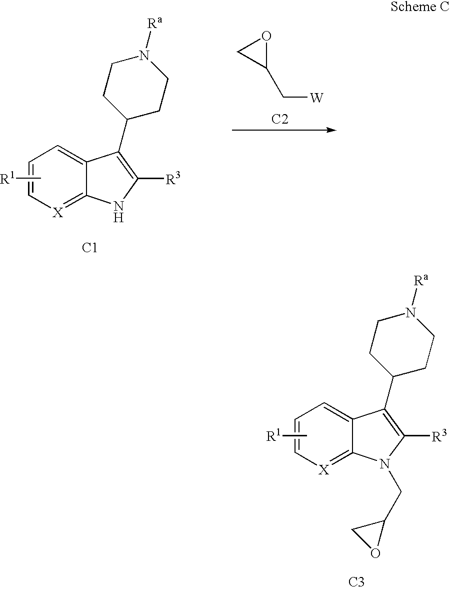

Compound C1 (wherein R^{a} is PG or R⁵) is reacted with a solution of Compound C2 (wherein W is a suitable leaving group such as Cl, Br, I, tosylate, mesylate and the like in an organic solvent such as NMP, DMF, THF and the like) in the presence of a base (such as NaH, KO-t-Bu, K₂CO₃, NaHMDS, LiHMDS and the like) to yield a Compound C3.

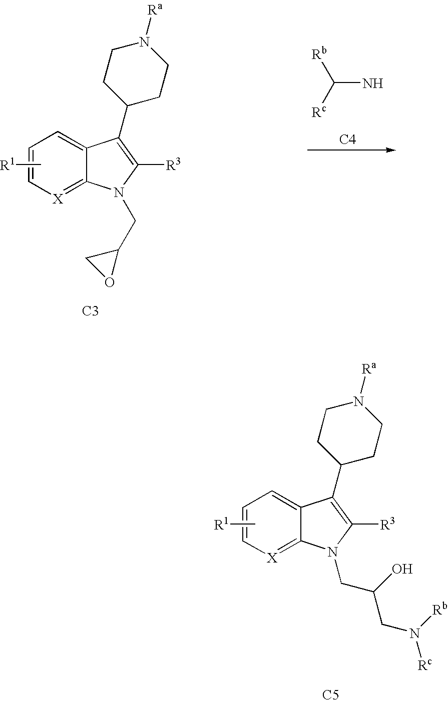

Compound C3 is reacted with a solution of Compound C4 (in an organic solvent such as methanol, ethanol, isopropanol, acetonitrile, THF and the like) to yield a Compound C5.

Compounds representative of formula (I) may be prepared according to the process of Scheme D.

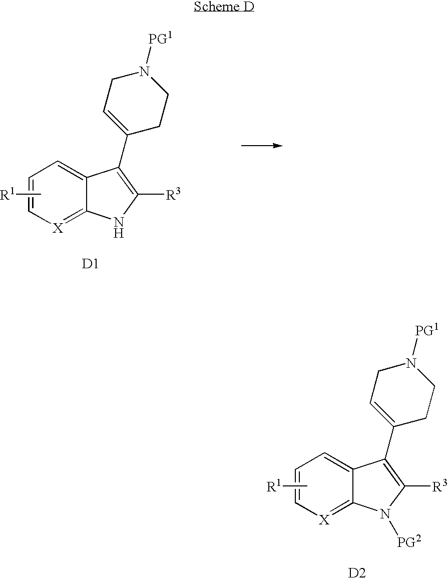

A solution of Compound D1 (wherein PG¹ is a suitable nitrogen protecting group in an organic solvent such as DCM, THF and the like) is reacted with an anhydride (such as Boc, CBz, Fmoc, benzhydryl, triphenylmethyl, 4-methoxybenzyl, benzoyl and the like) in the presence of a base (such as pyridine, TEA, DIPEA and the like) to yield a Compound D2 (wherein PG² is a suitable nitrogen protecting group).

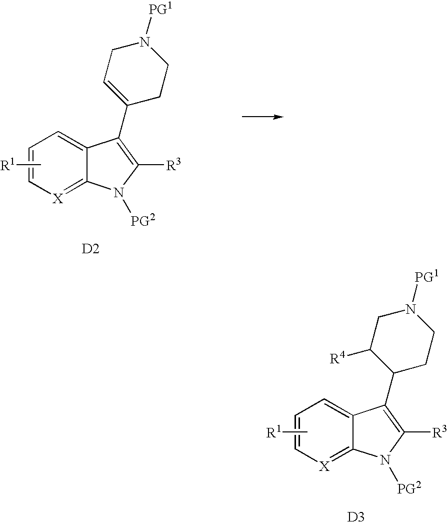

A solution of Compound D2 (in an organic solvent such as THF, DMF, NMP and the like) is reacted with a boranated reagent (such as borane dimethyl sulfide, a borane-THF complex, a sodium borohydride/boron trifluoride-diethyl etherate and the like) in the presence of a base (such as sodium hydroxide/hydrogen peroxide, potassium hydroxide, oxone, water and the like) to yield a Compound D3 (wherein R⁴ is present).

Alternatively, a solution of Compound D2 (in an organic solvent such as t-BuOH/water and the like) is reacted with a catalyst (such as osmium tetroxide and the like), then reduced with a solution of a reducing agent (such as Raney nickel and the like in an organic solvent such as EtOH, MeOH and the like) to yield Compound D3.

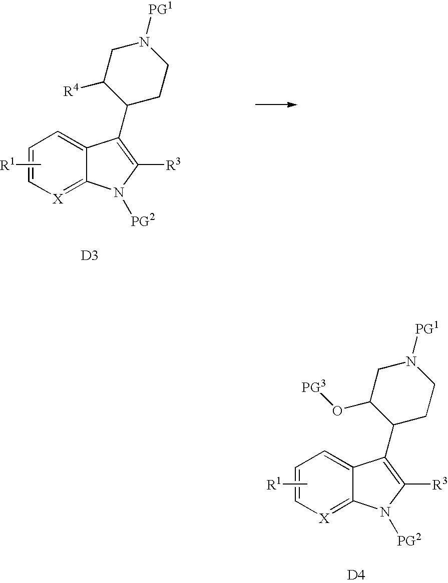

A solution of Compound D3 (in an organic solvent such as DCM, THF and the like) is reacted with an anhydride (such as acetic anhydride, isobutyric anhydride, benzoic anhydride and the like) in the presence of a base (such as pyridine, TEA, DIPEA and the like) to yield a Compound D4 (wherein the O-PG³ moiety is coextensive with R⁴ and wherein PG³ is a suitable oxygen protecting group).

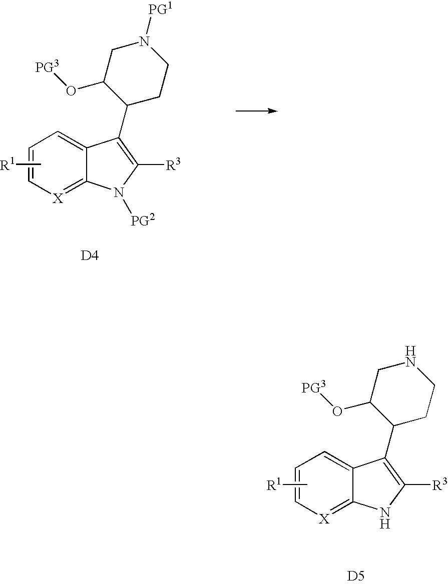

Compound D4 may be deprotected using techniques known to those skilled in the art to yield a Compound D5. For example, when either or both PG¹ and PG² are Boc, Compound D4 is reacted with an acid (such as TFA, HCl and the like).

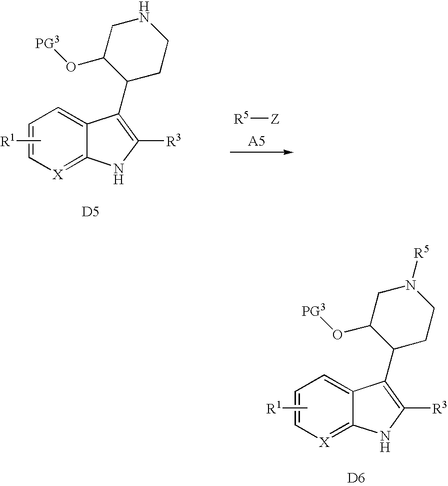

Using the procedure of Scheme A, Compound D5 is reacted with a solution of Compound A5 (wherein Z is a suitable leaving group such as Cl, Br, I, tosylate, mesylate and the like in an organic solvent such as DMF, DMSO, NMP and the like) in the presence of a base (such as TEA, DIPEA, pyridine, Na₂CO₃, K₂CO₃ and the like), wherein the base is present in an amount equal to or greater than about one molar equivalent of Compound A5, to yield a Compound D6.

For example, when R⁵-Z is an aldehyde or a ketone, Compound D5 is reacted with a solution of Compound A5 in the presence of a reducing agent (such as NaBH(OAc)₃, Na(BH₃)CN and the like) to yield Compound D6.

For example, when R⁵-Z is a carboxylic acid or acid chloride, Compound D5 is reacted with a solution of Compound A5 in the presence of a coupling agent (such as HATU, DCC and the like) to yield Compound D6.

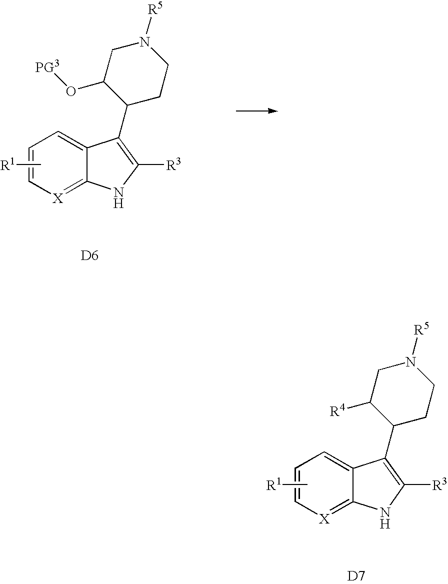

Compound D6 may be deprotected using techniques known to those skilled in the art to yield a Compound D7. For example, when the PG³ protecting group is acyl, Compound D6 is reacted with a base (such as MeONa, NaOH and the like).

The following Examples are set forth to aid in the understanding of the invention and are not intended and should not be construed to limit in any way the invention set forth in the claims which follow thereafter.

### EXAMPLE 1

### 5-fluoro-3-(1-naphthalen-1-ylmethyl-piperidin-4-yl)-1H-indole (Cpd 1)

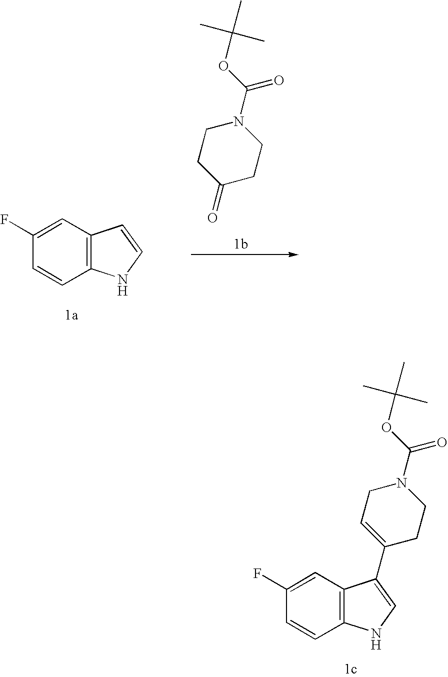

5-fluoro-1H-indole Compound 1a (5.0 g, 36.9 mmol) and 4-oxo-piperidine-1-carboxylic acid tert-butyl ester Compound 1b (14.74 g, 73.9 mmol) were dissolved in methanol. Potassium hydroxide (8.3 g, 148 mmol) was added under nitrogen atmosphere and the mixture was heated to reflux for 16 hours. The reaction mixture was then partitioned with icy water and methanol/DCM (10/90). The organic layer was washed with brine, dried with Na₂SO₄, then filtered and the solvent evaporated in vacuo to yield a crude foam. The crude foam was recrystallized from ethyl acetate/Hexanes to yield 4-(5-fluoro-1H-indol-3-yl)-3,6-dihydro-2H-pyridine-1-carboxylic acid tert-butyl ester Compound 1c as a solid.

¹H NMR (400 MHz, CDCl₃) δ 8.3 (1H, br s), 7.51 (1H, dd, J=2.39 Hz & 10.25 Hz), 7.29 (1H, dd, J=4.5 Hz & 8.8 Hz), 7.21-7.20 1H, m), 6.99-6.94 (1H, m), 6.07 (1H, br s), 4.13 (2H, m), 3.68-3.65 (2H, m), 2.53-2.54 (1H, m), 1.5 (9H, s); MS (ES⁺) m/z 339.2 (MNa)⁺

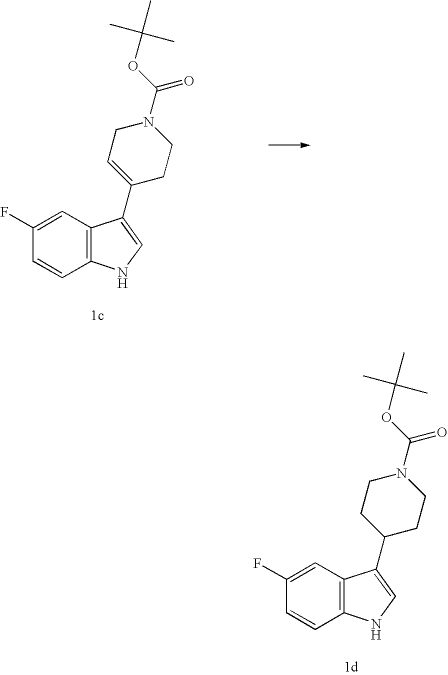

Compound 1c was dissolved in absolute ethanol (105 mL) and the mixture was hydrogenated (H₂, 60 psi) in a Parr instrument using platinum dioxide (0.9 g) as the catalyst. After 24 hours, the reaction mixture was filtered through a pad of celite and the solvent evaporated in vacuo to yield 4-(5-fluoro-1H-indol-3-yl)-piperidine-1-carboxylic acid tert-butyl ester Compound 1d as a solid.

¹H NMR (400 MHz, CDCl₃) δ 8.0 (1H, br s), 7.28-7.24 (2H, m), 7.0-6.99 (1H, m), 6.96-6.91 (1H, m), 4.22 (2H, br s), 2.93-2.87 (3H. m), 2.02-1.99 (2H, m), 1.64-1.57 (2H), 1.48 (9H, s); MS (ES⁺) m/z 337.4 (MNa)⁺

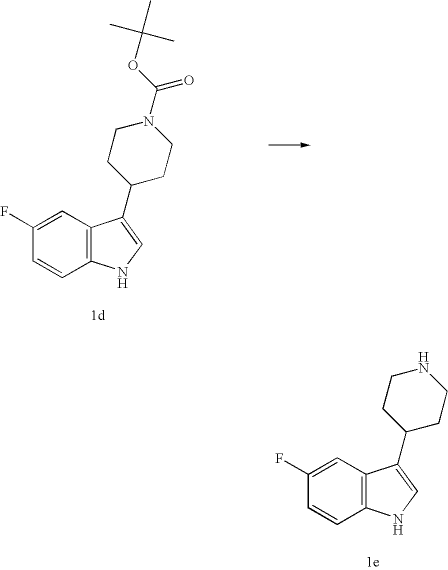

Compound 1d (4.0 g, 12.56 mmol) was dissolved in dry methylene chloride (60 mL). TFA (20 mL) was added at 0° C. and the mixture was stirred at 0° C. for 2 hours. The reaction mixture was then partitioned with aqueous sodium bicarbonate and DCM. The organic layer was washed with brine, dried with Na₂SO₄, filtered and the solvent evaporated in vacuo to yield 5-fluoro-3-piperidin-4-yl-1H-indole Compound 1e as a foam. MS (ES⁺) m/z 219.1 (MH)⁺

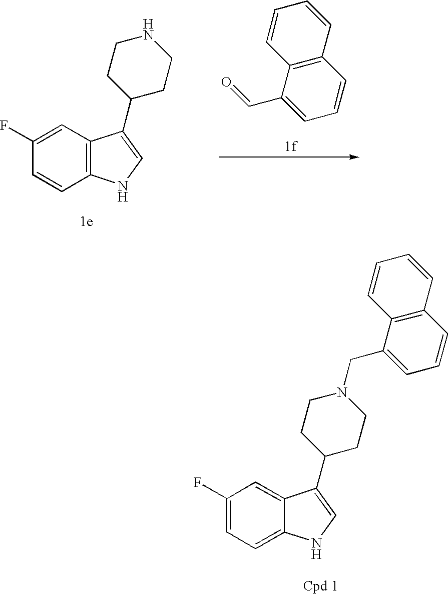

Compound 1e (0.12 g, 0.55 mmol) and naphthalene-1-carbaldehyde Compound 1f (0.103 g, 0.66 mmol) were dissolved in dry THF (4 mL) and dry 1,2-dichloroethane (1.0 mL). Sodium triacetoxyborohydride (0.174 g, 0.82 mmol) was added at room temperature under a nitrogen atmosphere and the mixture was stirred at room temperature for 18 hours. The reaction mixture was partitioned with aqueous 0.5N sodium hydroxide and DCM. The organic layer was washed with brine, dried with Na₂SO₄, then filtered and the solvent evaporated in vacuo to yield a crude oil. The crude oil was purified via flash chromatography (2.0% methanol/DCM) to yield Compound 1 as a foam.

¹H NMR (400 MHz, CDCl₃) δ 8.35 (1H, d, J=8.4 Hz), 7.89 (1H, br s), 7.86-7.77 (2H, m), 7.55-7.40 (4H, m), 7.28-7.22 (1H, m), 6.98 (1H, br s), 6.94-6.89 (2H, m), 3.96 (2H, s), 3.09-3.06 (2H, m), 2.82-2.74 (2H. m), 2.27-2.21 (2H, m), 2.0-1.97 (2H, m), 1.82-1.72 (2H, m); MS (ES⁺) m/z 359.1 (MH)⁺

### EXAMPLE 2

### 5-fluoro-3-[1-(8-methyl-naphthalen-1-ylmethyl)-piperidin-4-yl]-1H-indole (Cpd 2)

The title compound was prepared according to the procedure of Example 1, using 8-methyl-naphthalene-1-carbaldehyde Compound 2a to yield Compound 2 as a solid.

¹H NMR (400 MHz, CDCl₃) δ 7.88 (1H, br s), 7.78 (1H, dd, J=1.49 Hz & 7.98 Hz), 7.71-7.69 (1H, m), 7.41-7.40 (1H, m), 7.37-7.35 (1H, m), 7.33-7.32 (1H, m), 7.27-7.22 (2H, m), 6.96 (1H, br s), 6.93-6.88 (1H, m), 3.99 (2H, s), 3.13 (3H, s), 3.01-2.98 (2H, m), 2.80-2.4 (1H, m), 2.21-2.15 (2H, m), 1.98-1.94 (2H, m), 1.75-1.65 (2H, m); MS (ES⁺) m/z 373.2 (MH)⁺

### EXAMPLE 3

### 3-(1-cyclooctylmethyl-piperidin-4-yl)-5-fluoro-1H-indole (Cpd 3)

The title compound was prepared according to the procedure of Example 1, using cyclooctanecarbaldehyde Compound 3a (Tetrahedron 2001, 57(6), 981-986) to yield Compound 3 as a gum.

¹H NMR (400 MHz, CDCl₃) δ 7.95 (1H, br s), 7.29-7.24 (2H, m), 7.01 (1H, br s), 6.94-6.89 (1H, m), 3.0-2.97 (2H, m), 2.77-2.69 (1H, m), 2.15-1.97 (6H, m), 1.84-1.44 (15H, m), 1.27-1.18 (2H, m); MS (ES⁺) m/z 343.2 (MH)⁺

### EXAMPLE 4

### 3-(1-acenaphthen-1-yl-piperidin-4-yl)-5-fluoro-1H-indole (Cpd 4)

Acenaphthen-1-ol Compound 4a (88 mmol) was dissolved in diethyl ether (150 mL) and cooled to 0° C. Phosphorous tribromide (3.2 mL, 35 mmol) was then added slowly under nitrogen atmosphere. The reaction mixture was stirred for 30 minutes at room temperature and cooled to 0° C. The reaction mixture was partitioned with water and diethyl ether. The organic layer was dried over Na₂SO₄, filtered and the solvent evaporated in vacuo to yield 1-bromo-acenaphthene Compound 4b as a yellow solid.

Compound 4b (0.258 g, 1.1 mmol) and 5-fluoro-3-piperidin-4-yl-1H-indole Compound 1e (0.121 g, 0.55 mmol) were dissolved in DMF (4 mL). Potassium carbonate (0.23 g, 1.66 mmol) and a catalytic amount of potassium iodide were added and the mixture was stirred at 45° C. under a nitrogen atmosphere for 18 hours. The reaction mixture was partitioned with water and ethyl acetate. The organic layer was washed with brine, dried with Na₂SO₄, filtered and the solvent evaporated in vacuo to yield a crude oil. The crude oil was purified via flash chromatography (3.5% methanol/DCM) to yield Compound 4 as a solid.

¹H NMR (400 MHz, CDCl₃) δ 8.02 (1H, s), 7.95 (1H, br s), 7.72-7.68 (1H, m), 7.65-7.62 (1H, m), 7.60-7.52 (2H, m), 7.49-7.44 (1H, m), 7.31-7.22 (1H, 7.02-7.01 (1H, m), 6.99-6.89 (1H, m), 4.99 (1H, t, J=5.5 Hz), 3.48 (2H, d, J=5.5 Hz), 3.05-2.95 (2H, m), 2.82-2.63 (1H, m), 2.38-2.29 (2H, m), 2.02-1.97 (2H, m), 1.86-1.74 (2H, m); MS (ES⁺) m/z 371.2 (MH)⁺

### EXAMPLE 5

### 3-(1-acenaphthen-1-yl-piperidin-4-yl)-1-benzyl-6-chloro-1H-indole (Cpd 5)

1-bromo-acenaphthene Compound 4b (0.057 g, 0.24 mmol) and 1-benzyl-6-chloro-3-piperidin-4-yl-1H-indole Compound 5a (0.04 g, 0.12 mmol) were dissolved in DMF (3 mL). Potassium carbonate (0.051 g, 0.37 mmol) and a catalytic amount of potassium iodide were added and the mixture was stirred at 45° C. under a nitrogen atmosphere for 18 hours. The reaction mixture was partitioned with water and ethyl acetate. The organic layer was washed with brine, dried with Na₂SO₄, then filtered and the solvent evaporated in vacuo to yield a crude oil. The crude oil was purified via flash chromatography (1% methanol/DCM) to yield Compound 5 as a solid.

¹H NMR (300 MHz, CDCl₃) δ 7.72-7.67 (1H, m), 7.64-7.61 (1H, m), 7.55-7.43 (4H, m), 7.32-7.23 (4H, m), 7.21 (1H, d, J=1.7 Hz), 7.08-7.01 (3H, m), 6.85 (1H, s), 5.19 (2H, s), 4.98-4.94 (1H, m), 3.45 (2H, d, J=5.5 Hz), 2.96-2.93 (1H, m), 2.81-2.71 (2H, m), 2.64-2.55 (1H, m), 2.36-2.27 (1H, m), 2.02-1.69 (2H, m); MS (ES⁺) m/z 477.1 (MH)⁺

### EXAMPLE 6

### 3-(1-acenaphthen-1-yl-piperidin-4-yl)-5-chloro-2-methyl-1H-indole (Cpd 6)

1-bromo-acenaphthene Compound 4b (0.076 g, 0.33 mmol) and 5-chloro-2-methyl-3-piperidin-4-yl-1H-indole Compound 6a (0.041 g, 0.16 mmol) were dissolved in DMF (3 mL). Potassium carbonate (0.051 g, 0.37 mmol) and a catalytic amount of potassium iodide were added and the mixture was stirred at 45° C. under a nitrogen atmosphere for 18 hours. The reaction mixture was partitioned with water and ethyl acetate. The organic layer was washed with brine, dried with Na₂SO₄, filtered and the solvent evaporated in vacuo to yield a crude oil. The crude oil was purified via flash chromatography (4% methanol/DCM) to yield Compound 6 as a solid.

¹H NMR (300 MHz, DMSO) δ 10.89 (1H, br s), 7.73 (1H, d, J=7.58 Hz), 7.65 (1H, d, J=8.20 Hz), 7.58-7.46 (4H, m), 7.33 (1H, d, J=6.71 Hz), 7.21 (1H, d, J=8.52 Hz), 6.94 (1H, dd, J=2.01 Hz & 8.50 Hz), 4.96-4.94 (1H, m), 3.45-3.35 (2, m), 3.17 (2H, d, J=5.21 Hz), 2.94-2.91 (1H, m), 2.70-2.42 (2H, m), 2.32 (3H, s), 2.07-1.93 (2H, m), 1.64-1.54 (2H, m); MS (ES⁺) m/z 401.2 (MH)⁺

### EXAMPLE 7

### 1-benzyl-6-chloro-3-(1-naphthalen-1-ylmethyl-piperidin-4-yl)-1H-indole (Cpd 7)

The title compound was prepared according to the procedure of Example 1, using naphthalene-1-carbaldehyde Compound if and 1-benzyl-6-chloro-3-piperidin-4-yl-1H-indole Compound 5a to yield Compound 7 as a solid.

¹H NMR (300 MHz, CDCl₃) δ 8.33 (1H, d, J=7.70 Hz), 7.86-7.83 (1H, d, J=7.35 Hz), 7.77 ( 1H, d, J=7.90 Hz), 7.55-7.38 (5H, m), 7.32-7.24 (2H, m) 7.21 (1H, d, J=1.70 Hz), 7.07-7.02 (3H, m), 6.83 (1H, s), 5.18 (2H, s), 3.94 (2H, s), 3.08-3.04 (2H, m), 2.87-2.77 (1H, m), 2.26-2.19 (2H, m), 2.00-1.96 (2H, m), 1.82-1.72 (2H, m); MS (ES⁺) m/z 465.1 (MH)⁺

### EXAMPLE 8

### 1-benzyl-6-chloro-3-[1-(8-methyl-naphthalen-1-ylmethyl)-piperidin-4-yl]-1H-indole (Cpd 8)

The title compound was prepared according to the procedure of Example 1, using 8-methyl-naphthalene-1-carbaldehyde Compound 2a and 1-benzyl-6-chloro-3-piperidin-4-yl-1H-indole Compound 5a to yield Compound 8 as an oil.

¹H NMR (300 MHz, CDCl₃) δ 7.78 (1H, dd, J=1.50 Hz & 7.80 Hz), 7.72-7.68 (1H, m), 7.53 (1H, d, J=8.50 Hz), 7.41-7.25 (6H, m), 7.21 (1H, d, J=1.70 Hz), 7.07-7.02 (3H, m), 6.82 (1H, s), 5.18 (2H, s), 3.98 (2H, s), 3.12 ( 3H, s), 2.99-2.96 (2H, m), 2.86-2.76 (1H, m), 2.20-2.13 (2H, m), 1.97-1.93 (2H, m), 1.74-1.58 (2H, m); MS (ES⁺) m/z 479.1 (MH)⁺

### EXAMPLE 9

### 1-benzyl-6-chloro-3-(1-cyclooctylmethyl-piperidin-4-yl)-1H-indole (Cpd 9)

The title compound was prepared according to the procedure of Example 1, using cyclooctanecarbaldehyde Compound 3a and 1-benzyl-6-chloro-3-piperidin-4-yl-1H-indole Compound 5a to yield Compound 9 as an oil.

¹H NMR (300 MHz, CDCl₃) δ 7.55 (1H, d, J=8.46 Hz), 7.33-7.27 (3H, m), 7.22 ( 1H, br s), 7.08-7.02 (3H, m), 6.87 (1H, s), 5.20 (2H, s), 3.01-2.97 (2H, m), 2.81-2.73 (1H, m), 2.16-1.96 (6H, m), 1.87-1.44 (15H, m), 1.28-1.19 (2H, m); MS (ES⁺) m/z 449.2 (MH)⁺

### EXAMPLE 10

### 5-chloro-2-methyl-3-(1-naphthalen-1-ylmethyl-piperidin-4-yl)-1H-indole (Cpd 10)

The title compound was prepared according to the procedure of Example 1, using naphthalene-1-carbaldehyde Compound if and 5-chloro-2-methyl-3-piperidin-4-yl-1H-indole Compound 6a to yield Compound 10 as an oil.

¹H NMR (300 MHz, CDCl₃) δ 8.37-8.35 (1H, m), 7.89-7.86 (1H, m), 7.83-7.72 (2H, m), 7.63-7.42 (4H, m), 7.14 (1H, d, J=8.5 Hz), 7.01 (1H, dd, J=1.85 Hz & 8.50 Hz), 3.99 (2H, bs), 3.48 (2H, s), 3.25-3.04 (2H, m), 2.73-2.62 (1H, m), 2.39 (3H, s), 2.25-2.10 (4H, m), 1.71-1.57 (2H, m); MS (ES⁺) m/z 389.2 (MH)⁺

### EXAMPLE 11

### 5-chloro-2-methyl-3-[1-(8-methyl-naphthalen-1-ylmethyl)-piperidin-4-yl]-1H-indole (Cpd 11)

The title compound was prepared according to the procedure of Example 1, using 8-methyl-naphthalene-1-carbaldehyde Compound 2a and 5-chloro-2-methyl-3-piperidin-4-yl-1H-indole Compound 6a to yield Compound 11 as an oil.

¹H NMR (300 MHz, CDCl₃) δ 7.79 (1H, dd, J=1.5 Hz & 7.8 Hz), 7.73-7.70 (2H, m), 7.59 (1H, d, J=1.70 Hz), 7.43-7.33 (3H, m), 7.15-7.12 (1H, m), 7.00 (1H, dd, J=1.9 Hz & 8.5 Hz), 3.99 (2H, s), 3.17 (3H, s), 3.08-3.02 (2H, m), 2.77-2.68 (1H, m), 2.38 (3H, s), 2.24-2.04 (4H, m), 1.68-1.65 (2H, m); MS (ES⁺) m/z 403.2 (MH)⁺

### EXAMPLE 12

### 5-chloro-3-(1-cyclooctylmethyl-piperidin-4-yl)-2-methyl-1H-indole (Cpd 12)

The title compound was prepared according to the procedure of Example 1, using cyclooctanecarbaldehyde Compound 3a and 5-chloro-2-methyl-3-piperidin-4-yl-1H-indole Compound 6a to yield Compound 12 as an oil.

¹H NMR (300 MHz, CDCl₃) δ 7.75 (1H, br s), 7.66 (1H, d, J=1.60 Hz), 7.14 (1H, d, J=8.50 Hz), 7.02 (1H, dd, J=1.60 Hz & 8.50 Hz), 3.03-2.99 (2H, m), 2.72-2.61 (1H, m), 2.38 (3H, s), 2.25-2.12 (4H, m), 2.03-1.95 (2H, m), 1.78-1.49 (15H, m), 1.30-1.20 (2H); MS (ES⁺) m/z 373.4 (MH)⁺

### EXAMPLE 13

### 7-chloro-3-(1-naphthalen-1-ylmethyl-piperidin-4-yl)-1H-indole (Cpd 13)

The title compound was prepared according to the procedure of Example 1, using naphthalene-1-carbaldehyde Compound 1f and 7-chloro-3-piperidin-4-yl-1H-indole Compound 13a to yield Compound 13 as a foam.

¹H NMR (300 MHz, CDCl₃) δ 8.35 (1H, d, J=8.20 Hz), 8.18 (1H, br s), 7.85 (1H, dd, J=1.50 Hz & 7.90 Hz), 7.78 (1H, d, J=8.06 Hz), 7.55-7.39 (5H, m), 7.16 (1H, d, J=7.55 Hz), 7.03 (1H, d, J=7.80 Hz), 6.99-6.96 (1H, m), 3.99 (2H, s), 3.12 (3H, s), 3.10-3.06 (2H,m), 2.88-2.77 (1H,m), 2.29-2.20 (2H,m), 2.01-1.97 (2H, m), 1.86-1.72 (2H, m); MS (ES⁺) m/z 373.2 (MH)⁺

### EXAMPLE 14

### 7-chloro-3-[1-(8-methyl-naphthalen-1-ylmethyl)-piperidin-4-yl]-1H-indole (Cpd 14)

The title compound was prepared according to the procedure of Example 1, using 8-methyl-naphthalene-1-carbaldehyde Compound 2a and 7-chloro-3-piperidin-4-yl-1H-indole Compound 13a to yield Compound 14 as a foam.

¹H NMR (300 MHz, CDCl₃) δ 8.25 (1H, bs,), 7.77 (1H, dd, J=1.50 Hz & 7.90 Hz), 7.71-7.68 (1H, m), 7.53 (1H, d, J=7.90 Hz), 7.42-7.32 (3H, m), 7.16 (1H, d, J=7.25 Hz), 7.03-6.98 (1H, m), 6.95 (1H, d, J=2.20 Hz), 3.99 (2H, s), 3.12 (3H, s), 3.01-2.98 (2H, m), 2.87-2.81 (1H, m), 2.22-2.14 (2H, m), 1.98-1.94 (2H, m), 1.79-1.65 (2H, m); MS (ES⁺) m/z 389.2 (MH)⁺

### EXAMPLE 15

### 7-chloro-3-(1-cyclooctylmethyl-piperidin-4-yl)-1H-indole (Cpd 15)

The title compound was prepared according to the procedure of Example 1, using cyclooctanecarbaldehyde Compound 3a and 7-chloro-3-piperidin-4-yl-1H-indole Compound 13a to yield Compound 15 as an oil.

¹H NMR (300 MHz, CDCl₃) δ 8.25 (1H, bs), 7.55 (1H, d, J=7.90 Hz), 7.18-7.15 (1H, m), 7.08-6.99 (2H, m), 3.00-2.97 (2H, m), 2.83-2.73 (1H, m), 2.16-1.90 (5H, m), 1.87-1.44 (17H, m), 1.28-1.21 (2H, m); MS (ES⁺) m/z 359.2 (MH)⁺

### EXAMPLE 16

### 3-(1-acenaphthen-1-yl-piperidin-4-yl)-7-chloro-1H-indole (Cpd 16)

The title compound was prepared according to the procedure of Example 4, using 1-bromo-acenaphthene Compound 4b and 7-chloro-3-piperidin-4-yl-1H-indole Compound 13a to yield Compound 16 as a foam.

¹H NMR (300 MHz, CDCl₃) δ 8.21 (1H, bs,), 7.73-7.67 (1H, m), 7.63 ( 1H, d, J=8.2 Hz), 7.55-7.44 (3H, m), 7.31-7.28 (1H, m), 7.17-7.28 (1H, m), 7.05-6.97 (2H, m), 4.98 (1H, t, J=5.50 Hz), 3.47 (2H, d, J=5.50 Hz), 2.98-2.93 (1H, m), 2.84-2.72 (2H, m), 2.66-2.58 (1H, m), 2.37-2.28 (1H, m), 2.03-1.97 (2H, m), 1.92-1.74 (2H, m); MS (ES⁺) m/z 387.1 (MH)⁺

### EXAMPLE 17

### (1S)-3-(1-cyclooctylmethyl-piperidin-4-yl)-5-fluoro-oxiranylmethyl-1H-indole (Cpd 17)

3-(1-cyclooctylmethyl-piperidin-4-yl)-5-fluoro-1H-indole Compound 3 (0.087 g, 0.254 mmol) was dissolved in DMF (3 mL). Sodium hydride (60% in mineral oil, 0.012 g, 0.30 mmol) was added at 0° C. under nitrogen atmosphere and the mixture was stirred at 0° C. for 30 minutes. (2R)-(-)-glycidyl-3-nitrobenzene Compound 17a (0.079 g, 0.30 mmol) was added and the mixture was stirred at 0° C. for one hour, then for 18 hours at room temperature under nitrogen atmosphere. The reaction mixture was then partitioned with water and ethyl acetate. The organic layer was dried with Na₂SO₄, filtered and the solvent evaporated in vacuo to yield a crude oil. The crude oil was purified via flash chromatography (3% methanol/DCM) to yield Compound 17 as a foam.

¹H NMR (400 MHz, CDCl₃) δ 7.28-7.23 (2H, m), 6.97-6.92 (2H, m), 4.36 (1H, dd, J=3.00 Hz & 15.30 Hz), 4.08 (1H, dd, J=5.50 Hz & 15.30 Hz), 3.26-3.22 (1H, m), 3.00-2.98 (2H, m), 2.81-2.69 (m, 2H), 2.46-2.45 (1H, m), 2.26-1.96 (6H, m), 1.83-1.44 (15H, m), 1.27-1.20 (2H, m); MS (ES⁺) m/z 399.3 (MH)⁺

### EXAMPLE 18

### (1S)-5-fluoro-3-[1-(8-methyl-naphthalen-1-ylmethyl)-piperidin-4-yl]-oxiranylmethyl-1H-indole (Cpd 18)

The title compound was prepared according to the procedure of Example 17, using 5-fluoro-3-[1-(8-methyl-naphthalen-1-ylmethyl)-piperidin-4-yl]-1H-indole Compound 2 to yield Compound 18 as a gum.

¹H NMR (400 MHz, CDCl₃) δ 7.78 (1H, dd, J=1.37 Hz & 7.99 Hz), 7.72-7.68 (1H, m), 7.41-7.31 (4H, m), 7.26-7.21 (2H, m), 6.96-6.91 (1H, m), 6.88 (1H, s), 4.33 (1H, dd, J=3.03 Hz & 15.30 Hz), 4.04 (1H, dd, J=5.50 Hz & 15.30 Hz), 3.98 (2H, s), 3.24-3.20 (1H, m), 3.13 (3H, s), 3.00-2.97 (1H, m), 2.79-2.71 (2H, m), 2.44-2.43 (1H, m), 2.20-2.04 (2H, m), 1.96-1.92 (1H, m), 1.74-1.64 (2H, m); MS (ES⁺) m/z 429.3 (MH)⁺

### EXAMPLE 19

### (1R)-1-[3-(1-cyclooctylmethyl-piperidin-4-yl)-5-fluoro-indol-1-yl]-3-dimethylamino-propan-2-ol (Cpd 19)

(1S)-3-(1-cyclooctylmethyl-piperidin-4-yl)-5-fluoro-oxiranylmethyl-1H-indole Compound 17 (0.012 g, 0.030 mmol) was dissolved in methyl alcohol (1 mL). A solution of 2.0M dimethylamine in MeOH (0.3 mL) was added and the mixture was stirred at 45° C. for 12 hrs in a pressure flask. The solvent was then evaporated in vacuo to yield a crude oil. The crude oil was purified via flash chromatography (3.0% ammonia 2.0 M in methanol/DCM) to yield Compound 19 as a foam.

¹H NMR (400 MHz, CDCl₃) δ 7.26-7.23 (2H, m), 6.98 (1H, s), 6.95-6.90 (1H, m), 4.11-4.06 (2H, m), 4.03-3.94 (1H, m), 3.03-3.00 (2H, m), 2.76-2.70 (1H, m), 2.31-1.98 (15H, m), 1.84-1.45 (15H, m), 1.28-1.21 (2H, m); MS (ES⁺) m/z 444.3 (MH)⁺

### EXAMPLE 20

### (1R)-1-dimethylamino-3-{5-fluoro-3-[1-(8-methyl-naphthalen-1-ylmethyl)-piperidin-4-yl]-indol-1-yl}-propan-2-ol (Cpd 20)

The title compound was prepared according to the procedure of Example 19 using (1S)-5-fluoro-3-[1-(8-methyl-naphthalen-1-ylmethyl)-piperidin-4-yl]-oxiranylmethyl-1H-indole Compound 18 to yield Compound 20 as a gum.

¹H NMR (400 MHz, CDCl₃) δ 7.78 (1H, dd, J=1.46 Hz & 8.03 Hz), 7.72-7.69 (1H, m), 7.42-7.32 (4H, m), 7.25-7.21 (2H, m), 6.94-6.88 (1H, m), 4.08-4.04 (2H, m), 3.99-3.93 (3H, m), 3.12 (3H, s), 3.00-2.97 (1H, m), 2.79-2.72 (2H, m), 2.31-2.14 (11H, m), 1.97-1.93 (2H, m), 1.75-1.65 (2H, m); MS (ES⁺) m/z 474.2 (MNa)⁺

### EXAMPLE 21

### 4-(6-fluoro-1H-indol-3-yl)-3,6-dihydro-2H-pyridine-1-carboxylic acid tert-butyl ester (Cpd 47)

### 4-(6-fluoro-1H-indol-3-yl)-piperidine-1-carboxylic acid tert-butyl ester (Cpd 21)

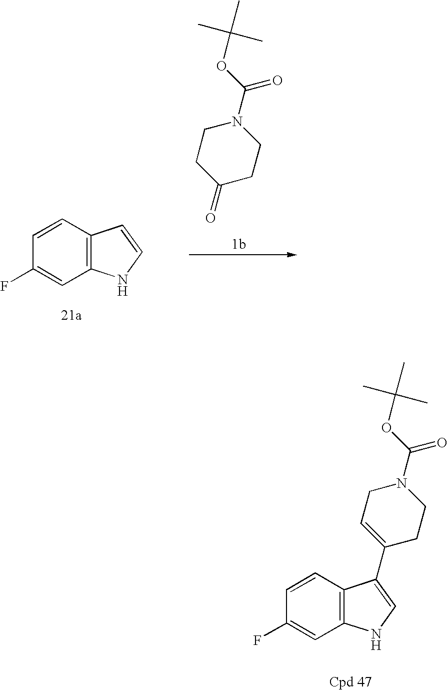

The title compound was prepared according to the procedure of Example 1, using 6-fluoro-1H-indole Compound 21a to yield Compound 47.

¹H NMR (300 MHz, CDCl₃) δ 8.13 (1H, br s), 7.78 (1H, dd, J=5.30 Hz & 8.80 Hz), 7.15 (1H, J=2.30 Hz), 7.05 (1H, dd, J=2.30 Hz & 9.40 Hz), 6.95-6.89 (1H, m), 6.13 (1H, br s), 4.13-4.12 (2H, m), 3.67 (2H, t, J=5.60 Hz), 2.55 (2H, br s); MS (ES⁺) m/z340.1 (MNa)⁺

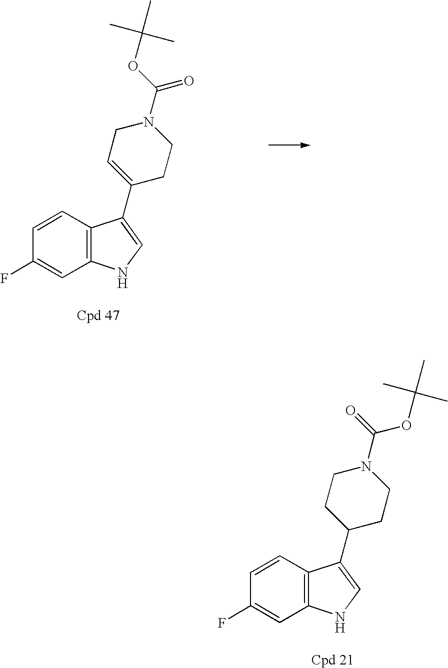

The title compound was prepared according to the procedure of Example 1, using Compound 47 to yield Compound 21 as a solid.

¹H NMR (300 MHz, CDCl₃) δ 8.05 (1H, br s), 7.52 (1H, dd, J=5.30 Hz & 8.70 Hz), 7.04 (1H, dd, J=2.20 Hz & 9.60 Hz), 6.92-6.83 (1H, m), 4.22 (2H, br s), 2.99-2.84 (3H, m), 2.03-1.99 (2H, m), 1.70-1.56 (2H, m), 1.48 (9H, s); MS (ES⁺) m/z 341.1 (MNa)⁺

### EXAMPLE 22

### 3-(1-acenaphthen-1-yl-piperidin-4-yl)-6-fluoro-1H-indole (Cpd 22)

The title compound was prepared according to the procedure of Example 4, using 4-(6-fluoro-1H-indol-3-yl)-piperidine-1-carboxylic acid tert-butyl ester Compound 21 to yield Compound 22 as a foam.

¹H NMR (300 MHz, CDCl₃) δ 7.92 (1H, br s), 7.72-7.69 (1H, m), 7.63 (1H, d, J=8.20 Hz), 7.54-7.44 (4H, m), 7.30 d, J=7.10 Hz), 7.02 (1H, dd, J=2.20 Hz & 9.60 Hz), 6.94-6.93 (1H, m), 6.88-6.81 (1H, m), 4.98 (1H, t, J=5.50 Hz), 3.47 (2H, d, J=5.50 Hz), 2.98-2.94 (1H, m), 2.81-2.76 (2H, m), 2.63-2.62 (1H, m), 2.33-2.32 (1H, m), 2.02-1.97 (2H, m), 1.86-1.76 (2H, m); MS (ES⁺) m/z 371.1 (MH)⁺

### EXAMPLE 23

### (1R)-1-[3-(1-acenaphthen-1-yl-piperidin-4-yl)-6-fluoro-indol-1-yl]-3-amino-propan-2-ol (Cpd 23)

3-(1-acenaphthen-1-yl-piperidin-4-yl)-6-fluoro-1H-indole Compound 22 (0.07 g, 0.19 mmol) was dissolved in DMF (2 mL). Sodium hydride (60% in mineral oil, 0.009 g, 0.23 mmol) was added at 0° C. under nitrogen atmosphere and the mixture was stirred at 0° C. for 30 minutes. (2R)-(-)-glycidyl-3-nitrobenzene Compound 17a (0.06 g, 0.25 mmol) was added and the mixture was stirred at 0° C. for one hour, then for 18 hours at room temperature under nitrogen atmosphere. The reaction mixture was then partitioned with water and ethyl acetate. The organic layer was dried with Na₂SO₄, filtered and the solvent evaporated in vacuo to yield a crude oil. The crude oil was purified via flash chromatography (3% methanol/DCM) to yield (1S)-3-(1-acenaphthen-1-yl-piperidin-4-yl)-6-fluoro-oxiranylmethyl-1H-indole Compound 23a as a foam.

Compound 23a (0.020 g, 0.046 mmol) was dissolved in a mixture of methyl alcohol/ethyl alcohol (1/1 mL). Concentrated ammonium hydroxide (0.3 mL) was added and the mixture was stirred at 50° C. for 12 hrs in a pressure flask. The solvent was evaporated in vacuo to yield a crude oil that was purified via semi-preparative reverse phase HPLC (0.5% TFA in acetonitrile/0.5% TFA in water using a YMC J'Sphere ODS-H80, 100×20 mm ID column) to yield Compound 23 as a foam.

¹H NMR (400 MHz, CDCl₃) δ 7.72-7.69 (1H, m), 7.63 (1H, d, J=8.20 Hz), 7.53-7.52 (2H, m), 7.50-7.44 (2H, m), 7.30 (1H, d, J=6.80 Hz), 7.01-6.98 (1H, m), 6.87 (1H, s), 6.85-6.78 (1H, m), 5.00-4.96 (1H, m), 4.04-3.97 (2H, m), 3.87 (1H, br s), 3.46 (2H, d, J=5.25 Hz), 2.97-2.94 (1H, m), 2.83-2.70 (2H, m), 2.65-2.58 (2H, m), 2.35-2.28 (1H, m), 2.04-1.98 (2H, m), 1.88-1.75 (3H,m); MS (ES⁺) m/z 444.1 (MH)⁺

### EXAMPLE 24

### (1R)-1-[3-(1-acenaphthen-1-yl-piperidin-4-yl)-6-fluoro-indol-1-yl]-3-methylamino-propan-2-ol (Cpd 24)

(1S)-3-(1-acenaphthen-1-yl-piperidin-4-yl)-6-fluoro-oxiranylmethyl-1H-indole Compound 23a (0.020 g, 0.046 mmol) was dissolved in methyl alcohol (1 mL). A solution of 2.0M methylamine in MeOH (0.3 mL) was added and the mixture was stirred at 50° C. for 12 hrs in a pressure flask. The solvent was then evaporated in vacuo to yield a crude oil. The crude oil was purified via semi-preparative reverse phase HPLC (0.5% TFA in acetonitrile/0.5% TFA in water using a YMC J'Sphere ODS-H80, 100×20 mm ID column) to yield Compound 24 as a foam.

¹H NMR (400 MHz, CDCl₃) δ 7.72-7.68 (1H, m), 7.63 (1H, d, J=8.20 Hz), 7.53-7.52 (2H, m), 7.49-7.44 (2H, m), 7.30 (1H, d, J=6.80 Hz), 7.00 (1H, dd, J=2.10 Hz & 10.00 Hz), 6.87 (1H, s), 6.85-6.78 (1H, m), 4.98 (1H, t, J=5.50 Hz), 4.02-3.98 (3H, m), 3.47 (2H, d, J=5.50 Hz), 3.08-2.94 (1H, m), 2.84-2.60 (5H, m), 2.58-2.46 (1H, m), 2.36-2.28 (1H, m), 2.00-1.97 (2H, m), 1.88-1.76 (2H, m); MS (ES⁺) m/z 458.1 (MH)⁺

### EXAMPLE 25

### (1R)-1-[3-(1-acenaphthen-1-yl-piperidin-4-yl)-6-fluoro-indol-1-yl]-3-dimethylamino-propan-2-ol (Cpd 25)

The title compound was prepared according to the procedure of Example 24, using (1S)-3-(1-acenaphthen-1-yl-piperidin-4-yl)-6-fluoro-oxiranylmethyl-1H-indole Compound 23a and 2.0M dimethylamine in MeOH to yield Compound 25 as a foam.

¹H NMR (300 MHz, CDCl₃) δ 7.74-7.71 (1H, m), 7.64 (1H, d, J=8.20 Hz), 7.57-7.51 (2H, m), 7.50-7.45 (2H, m), 7.31 (1H, d, J=6.80 Hz), 7.01 (1H, dd, J=2.20 Hz & 10.10 Hz), 6.90 (1H, s), 6.85-6.78 (1H, m), 5.05 (1H, br s), 4.04-3.93 (3H, m), 3.51 (2H, d, J=5.25 Hz), 3.04-3.00 (1H, m), 2.92-2.89 (1H, m), 2.81-2.65 (2H, m), 2.38-2.31 (2H, m), 2.24-2.19 (1H, m) 2.01-1.99 (2H, m), 1.91-1.83 (2H, m); MS (ES⁺) m/z 472.2 (MH)⁺

### EXAMPLE 26

### 3-piperidin-4-yl-1H-indole (Cpd 26)

The title compound was prepared according to the procedure of Example 1, using 1H-indole Compound 26a to yield Compound 26 as a solid. MS (ES⁺) m/z 201 (MH)⁺

### EXAMPLE 27

### 6-fluoro-3-piperidin-4-yl-1H-indole (Cpd 27)

The title compound was prepared according to the procedure of Example 1, using 4-(6-fluoro-1H-indol-3-yl)-piperidine-1-carboxylic acid tert-butyl ester Compound 21 to yield Compound 27 as a solid. MS (ES⁺) m/z 219.12 (MH)⁺

### EXAMPLE 28

### 1-benzyl-3-piperidin-4-yl-1H-indole (Cpd 28)

The procedures described in PCT Applications WO 02020013 and WO 02014317 were used to prepare Compound 28. MS m/z 291 (MH)⁺

### EXAMPLE 29

### 5-chloro-3-piperidin-4-yl-1H-indole (Cpd 29)

Using the procedure of Example 1 and 5-chloro-1H-indole Compound 29a yielded Compound 29 (as described in PCT Applications WO 02020013 and WO 02014317). MS m/z 235 (MH)⁺

### EXAMPLE 30

### 3-piperidin-4-yl-1H-pyrrolo[2,3-b]pyridine (Cpd 30)

The procedures described in PCT Applications WO 02020013 and WO 02014317 were used to prepare Compound 30. MS m/z 202 (M⁺H)

### EXAMPLE 31

### 3-[1-(8-methyl-naphthalen-1-ylmethyl)-piperidin-4-yl]-1H-pyrrolo[2,3-b]pyridine (Cpd 31)

The title compound was prepared according to the procedure of Example 1, using 8-methyl-naphthalene-1-carbaldehyde Compound 2a and 3-piperidin-4-yl-1H-pyrrolo[2,3-b]pyridine Compound 30 to yield Compound 31 as a solid.

¹H NMR (400 MHz, CDCl₃) δ 8.68 (1H, br s), 8.26 (1H, dd, J=1.30 Hz & 4.70 Hz), 7.93 (1H, dd, J=0.90 Hz & 7.90 Hz), 7.78 (1H, dd, J=1.50 Hz & 7.90 Hz), 7.72-7.69 (1H, m), 7.41 (1H, dd, J=1.50 Hz & 7.0 Hz), 7.36-7.32 (2H, m), 7.05-9.99 (2H, m), 3.99 (2H, s), 3.13 (3H, s), 3.01-2.98 (2H, m), 2.85-2.77 (1H, m), 2.21-2.15 (2H, m), 1.98-1.94 (2H, m), 1.79-1.69 (2H, m); MS (ES⁺) m/z 356.3 (MH)⁺

### EXAMPLE 32

### 3-(1-cyclooctylmethyl-piperidin-4-yl)-1H-pyrrolo[2,3-b]pyridine (Cpd 32)

The title compound was prepared according to the procedure of Example 1, using cyclooctanecarbaldehyde Compound 3a and 3-piperidin-4-yl-1H-pyrrolo[2,3-b]pyridine Compound 30 to yield Compound 32 as a solid.

¹H NMR (400 MHz, CDCl₃) δ 8.72 (1H, br s), 8.28 (1H, d, J=4.70 Hz), 7.96 (1H, d, J=7.75 Hz), 7.06-6.99 (2H, m), 2.99-2.96 (2H, m), 2.80-2.74 (1H, m), 2.13-1.97 (6H, m), 1.85-1.44 (15H, m), 1.26-1.21 (2H, m); MS (ES⁺) m/z 326.3 (MH)⁺

### EXAMPLE 33

### 3-(1-naphthalen-1-ylmethyl-piperidin-4-yl)-1H-pyrrolo[2,3-b]pyridine (Cpd 33)

The title compound was prepared according to the procedure of Example 1, using naphthalene-1-carbaldehyde Compound if and 3-piperidin-4-yl-1H-pyrrolo[2,3-b]pyridine Compound 30 to yield Compound 33 as a solid.

¹H NMR (400 MHz, CDCl₃) δ 8.73 (1H, br s), 8.35 (1H, d, J=8.10 Hz), 8.27-8.26 (1H, m), 7.95 (1H, d, J=7.85 Hz), 7.85 (1H, d, J=8.30 Hz), 7.78 (1H, d, J=7.98 Hz), 7.55-7.40 (3H, m), 7.05-6.00 (2H, m), 3.96 (2H, s), 3.09-3.07 (2H, m), 2.86-2.79 (1H, m), 2.27-2.21 (2H, m), 2.00-1.97 (2H, m), 1.86-1.76 (2H, m); MS (ES⁺) m/z 342.1 (MH)⁺

### EXAMPLE 34

### 3-(1-acenaphthen-1-yl-piperidin-4-yl)-1H-pyrrolo[2,3-b]pyridine (Cpd 34)

The title compound was prepared according to the procedure of Example 4, using 1-bromo-acenaphthene Compound 4b and 3-piperidin-4-yl-1H-pyrrolo[2,3-b]pyridine Compound 30 to yield Compound 34 as a solid.

¹H NMR (400 MHz, DMSO) δ 11.38 (1H, br s), 8.15 (1H, dd, J=1.40 Hz & 4.65 Hz), 7.95-7.93 (1H, m), 7.73 (1H, d, J=8.20 Hz), 7.66 (1H, d, J=8.15 Hz), 7.57-7.50 (1H, m), 7.48-7.46 (2H, m), 7.34-7.32 (1H, m), 7.20-7.19 (1H, m), 7.00-6.97 (1H, m), 4.94 (1H, br s), 3.39-3.36 (2H, m), 2.96-2.88 (2H, m), 2.76-2.65 (2H, m), 2.38-2.31 (2H, m), 1.96-1.82 (2H, m), 1.78-1.65 (2H, m); MS (ES⁺) m/z 354.3 (MH)⁺

### EXAMPLE 35

### 1-acenaphthen-1-yl-3-(1-acenaphthen-1-yl-piperidin-4-yl)-1H-pyrrolo[2,3-b]pyridine (Cpd 35)

1-bromo-acenaphthene Compound 4b (0.972 g, 4.18 mmol) and 3-piperidin-4-yl-1H-pyrrolo[2,3-b]pyridine Compound 30 (0.420 g, 2.08 mmol) were dissolved in DMF (12 mL). Potassium carbonate (0.865 g, 6.26 mmol) and a catalytic amount of potassium iodide were added and the mixture was stirred at 40° C. under a nitrogen atmosphere for 20 hours. The reaction mixture was partitioned with water and ethyl acetate. The organic layer was washed with brine, dried with Na₂SO₄, filtered and the solvent evaporated in vacuo to yield a crude oil. The crude oil was purified via flash chromatography (3.0% ammonia 2.0 M in methanol/DCM) to yield Compound 35 as an oil. MS (ES⁺) m/z 506.2 (MH)⁺

### EXAMPLE 36

### 3-(1-acenaphthen-1-yl-piperidin-4-yl)-1-methyl-1H-pyrrolo[2,3-b]pyridine (Cpd 36)

3-(1-acenaphthen-1-yl-piperidin-4-yl)-1H-pyrrolo[2,3-b]pyridine Compound 34 (0.047 g, 0.13 mmol) was dissolved in DMF (1.5 mL). Sodium hydride (60% in mineral oil, 0.0064 g, 0.16 mmol) was added to the mixture at 0° C. under a nitrogen atmosphere. The mixture was stirred at 0° C. for 30 minutes, then methyl iodide (0.028 g, 0.20 mmol) was added. The mixture was stirred at 0° C. for one hour, then for 18 hours at room temperature under nitrogen atmosphere. The reaction mixture was then partitioned with water and ethyl acetate. The organic layer was dried with Na₂SO₄, filtered and the solvent evaporated in vacuo to yield a crude oil. The crude oil was purified via flash chromatography (4.5% methanol/DCM) to yield Compound 36 as a gum.

¹H NMR (400 MHz, CDCl₃) δ 8.29 (1H, dd, J=1.50 Hz & 4.70 Hz), 7.89 (1H, dd, J=1.5 Hz & 7.86 Hz), 7.71-7.68 (1H, m), 7.63 (1H, d, J=8.25 Hz), 7.55-7.52 (2H, m), 7.48-7.44 (1H, m), 7.30 (1H, d, J=6.80 Hz), 6.99 (1H, dd, J=4.72 Hz & 7.85 Hz), 6.92 (1H, s), 4.99-4.96 (1H, m), 3.83 (3H, s), 3.48-3.46 (2H, m), 2.98-2.94 (1H, m), 2.87-2.72 (2H, m), 2.64-2.58 (1H, m), 2.36-2.29 (1H, m), 2.00-1.76 (4H, m); MS (ES⁺) m/z 368.3 (MH)⁺

### EXAMPLE 37

### 3-(1-acenaphthen-1-yl-piperidin-4-yl)-1-[2-(tetrahydro-pyran-2-yloxy)-ethyl]-1H-pyrrolo [2,3-b]pyridine (Cpd 37)

3-(1-acenaphthen-1-yl-piperidin-4-yl)-1H-pyrrolo[2,3-b]pyridine Compound 34 (0.052 g, 0.146 mmol) was dissolved in DMF (1.5 mL). Sodium hydride (60% in mineral oil, 0.007 g, 0.175 mmol) under a nitrogen atmosphere was added to the mixture at 0° C. The mixture was stirred at 0° C. for 30 minutes, then 2-(2-bromo-ethoxy)-tetrahydro-pyran (0.040 g, 0.19 mmol) was added. The mixture was stirred at 0° C. for one hour, then for 18 hours at room temperature under nitrogen atmosphere. The reaction mixture was partitioned with water and ethyl acetate. The organic layer was dried with Na₂SO₄, filtered and the solvent evaporated in vacuo to yield a crude oil. The crude oil was purified via flash chromatography (4% methanol/DCM) to yield Compound 37 as a gum.

¹H NMR (400 MHz, CDCl₃) δ 8.26 (1H, dd, J=1.50 Hz & 4.70 Hz), 7.88 (1H, d, J=7.80 Hz), 7.71-7.68 (1H, m), 7.63 (1H, d, J=8.10 Hz), 7.55-7.50 (2H, m), 7.48-7.44 (1H, m), 7.30 (1H, d, J=6.70 Hz), 7.12 (1H, s), 6.99 (1H, dd, J=4.70 Hz & 7.80 Hz), 4.99-4.97 (1H, m), 4.52-4.38 (3H, m), 4.05-4.00 (1H, m), 3.75-3.69 (1H, m), 3.58-3.52 (1H, m), 3.47-3.46 (2H, m), 3.37-3.32 (1H, m), 2.97-2.95 (1H, m), 2.88-2.71 (2H, m), 2.64-2.59 (1H, m), 2.35-2.29 (1H, m), 2.00-1.89 (2H, m), 1.86-1.72 (3H, m), 1.67-1.41 (5H, m); MS (ES⁺) m/z 482.4 (MH)⁺

### EXAMPLE 38

### 2-[3-(1-acenaphthen-1-yl-piperidin-4-yl)-pyrrolo[2,3-b]pyridin-1-yl]-ethanol (Cpd 38)

3-(1-acenaphthen-1-yl-piperidin-4-yl)-1-[2-(tetrahydro-pyran-2-yloxy)-ethyl]-1H-pyrrolo[2,3-b]pyridine Compound 37 (0.043 g, 0.09 mmol) was dissolved in methanol (2.5 mL) and aqueous 1.0N HCl (0.4 mL) was added at room temperature. The mixture was stirred for 1 hour at room temperature and 10 minutes at 60° C., then neutralized with an aqueous saturated solution of sodium carbonate. The reaction mixture was partitioned with water and DCM. The organic layer was dried with Na₂SO₄, then filtered and evaporated in vacuo to yield a crude oil. The crude oil was purified via flash chromatography (7% methanol/DCM) to yield Compound 38 as a gum.

¹H NMR (400 MHz, CDCl₃) δ 8.21 (1H, dd, J=1.48 Hz & 4.77 Hz), 7.92 (1H, dd, J=1.5 Hz & 7.89 Hz), 7.72-7.68 (1H, m), 7.63 (1H, d, J=8.25 Hz), 7.55-7.50 (2H, m), 7.46 (1H, dd, J=6.90 Hz & 8.20 Hz), 7.30 (1H, d, J=6.76 Hz), 7.03-7.00 (1H, m), 6.93 (1H, s), 4.99-4.96 (1H, m), 4.36-4.31 (2H, m), 4.03-3.99 (2H, m), 3.48-3.45 (2H, m), 2.97-2.94 (1H, m), 2.82-2.70 (2H, m), 2.63-2.56 (1H, m), 2.35-2.29 (1H, m), 1.99-1.75 (5H, m); MS (ES⁺) m/z 398.3 (MH)⁺

### EXAMPLE 39

### (3S)-3-(1-tert-butoxycarbonyl-3-hydroxy-piperidin-4-yl)-pyrrolo[2,3-b]pyridine-1-carboxylic acid tert-butyl ester (Cpd 41)

### acetic acid (4S)-4-(1H-pyrrolo[2,3-b]pyridin-3-yl)-piperidin-3-yl ester (Cpd 60)

Using the procedure of Example 1, 1H-pyrrolo[2,3-b]pyridine (3.0 g, 25.4 mmol) and 4-oxo-piperidine-1-carboxylic acid tert-butyl ester Compound 1b (4.2 g, 21.08 mmol) were dissolved in methanol (60 mL). Potassium hydroxide (3.56 g, 63.44 mmol) was added under a nitrogen atmosphere and the mixture was heated to reflux for 18 hours. The reaction mixture was partitioned with icy water and methanol/DCM (10/90). The organic layer was washed with brine and dried with Na₂SO₄, then filtered and evaporated in vacuo to yield 4-(1H-pyrrolo[2,3-b]pyridin-3-yl)-3,6-dihydro-2H-pyridine-1-carboxylic acid tert-butyl ester Compound 39a as a solid.

¹H NMR (400 MHz, CDCl₃) δ 9.95 (1H, br s), 8.34 (1H, dd, J=1.40 Hz & 4.70 Hz), 8.19 (1H, dd, J=1.30 Hz & 8.00 Hz), 7.32 (1H, s), 7.13 (1H, dd, J=4.70 Hz & 7.98 Hz), 6.14 (1H, br s), 4.15-4.14 (2H, m), 3.70-3.67 (2H, m), 2.57 (2H, br s), 1.50 (9H, s); MS (ES⁺) m/z 300.1 (MH)⁺

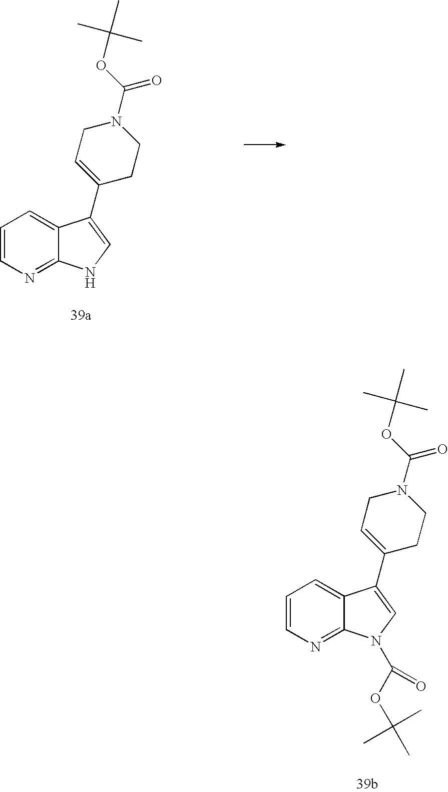

Triethylamine (1.01 g, 10.02 mmol) was added to a solution of Compound 39a (1.0 g, 3.34 mmol) in dry DCM (15 mL) and di-tert-butyl dicarbonate (1.6 g, 7.34 mmol) and dimethyl aminopyridine (0.49 g, 4.0 mmol) were added at 0° C. under a nitrogen atmosphere. The mixture was stirred at 0° C. for 15 minutes, then for 2.5 hours at room temperature. The reaction mixture was partitioned with saturated aqueous sodium bicarbonate and DCM. The organic layer was washed with water and brine and dried with Na₂SO₄, then filtered and evaporated in vacuo to yield a crude oil. The crude oil was purified via flash chromatography (2% methanol/DCM) to yield 3-(1-tert-butoxycarbonyl-1,2,3,6-tetrahydro-pyridin-4-yl)-pyrrolo [2,3-b]pyridine-1-carboxylic acid tert-butyl ester Compound 39b as a foam.

¹H NMR (300 MHz, CDCl₃) δ 8.52 (1H, dd, J=1.48 Hz & 4.75 Hz), 8.11 (1H, dd, J=1.50 Hz & 7.98 Hz), 7.55 (1H, s), 7.22 (1H, dd, J=4.78 Hz & 7.98 Hz), 6.19 (1H, br s), 4.14-4.13 (2H, m), 3.70-3.66 (2H, m), 2.54 (2H, br s), 1.67 (9H, s), 1.50 (9H, s); MS (ES⁺) m/z 400.1 (MH)⁺

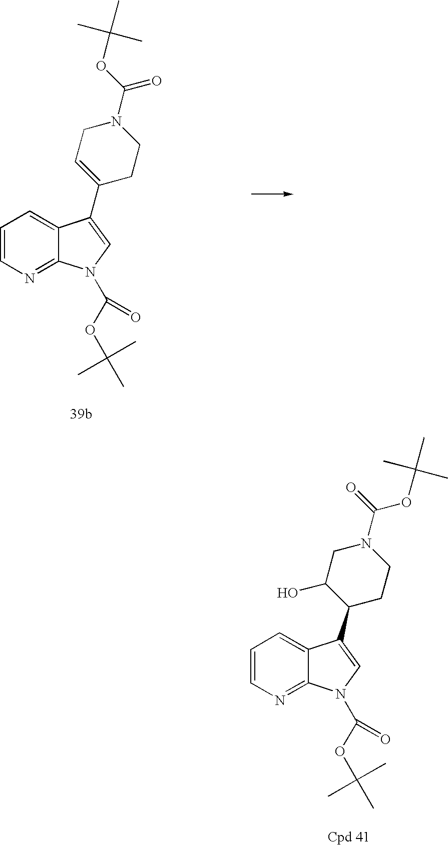

Compound 39b (0.25 g, 0.625 mmol) was dissolved in dry THF (4 ML) and a borane-methyl sulfide complex (125 μL of a 10 M solution, 1.25 mmol) was added at 0° C. The mixture was stirred at room temperature for 18 hours, then cooled to 0° C. and treated with aqueous 3N sodium hydroxide (0.4 mL) and hydrogen peroxide (0.24 mL of a 30 wt % solution in water). The mixture was stirred to room temperature for 18 hours, then partitioned with saturated aqueous sodium bicarbonate and ethyl acetate. The organic layer was washed with water and brine and dried with Na₂SO₄, then filtered and evaporated in vacuo to yield a crude oil. The crude oil was purified via flash chromatography (3% methanol/DCM) to yield Compound 41 as a gum.

¹H NMR (300 MHz, CDCl₃) δ 8.51 (1H, dd, J=1.40 Hz & 4.70 Hz), 7.95 (1H, dd, J=1.40 Hz & 7.86 Hz), 7.50 (1H, s), 7.19 (1H, dd, J=4.70 Hz & 7.90 Hz), 4.42 (1H, br s), 4.23 (1H, br s), 3.79 (1H, br s), 2.83-2.70 (3H, m), 1.92-1.83 (2H, m), 1.67 (9H, s), 1.50 (9H, s); MS (ES⁺) m/z 418.0 (MH)⁺

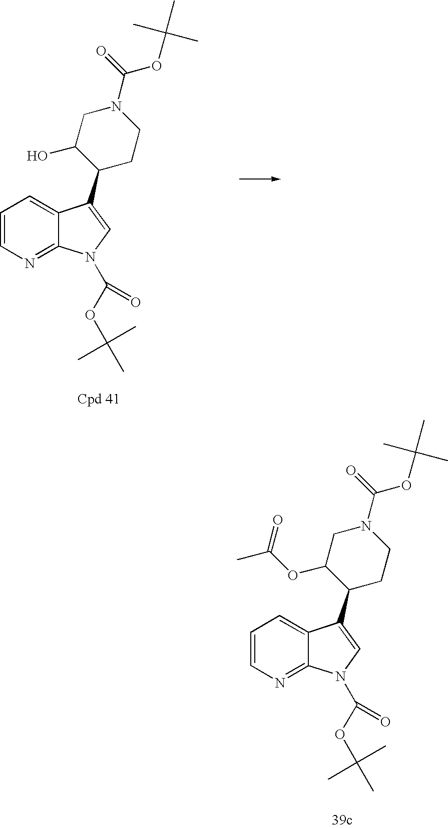

Compound 41 (0.2 g, 0.48 mmol) was dissolved in dry DCM (8 mL) and dry pyridine (2 mL). Acetic anhydride (0.49 g, 4.79 mmol) and a crystal of dimethyl aminopyridine were added at 0° C. The mixture was stirred to room temperature for 18 hours and then partitioned with water and DCM. The organic layer was washed with brine and dried with Na₂SO₄, then filtered and evaporated in vacuo to yield a crude oil. The crude oil was purified via flash chromatography (1% methanol/DCM) to yield 3-(3-acetoxy-1-tert-butoxycarbonyl-piperidin-4-yl)-pyrrolo [2,3-b]pyridine-1-carboxylic acid tert-butyl ester Compound 39c. MS (ES⁺) m/z 460.2 (MH)⁺

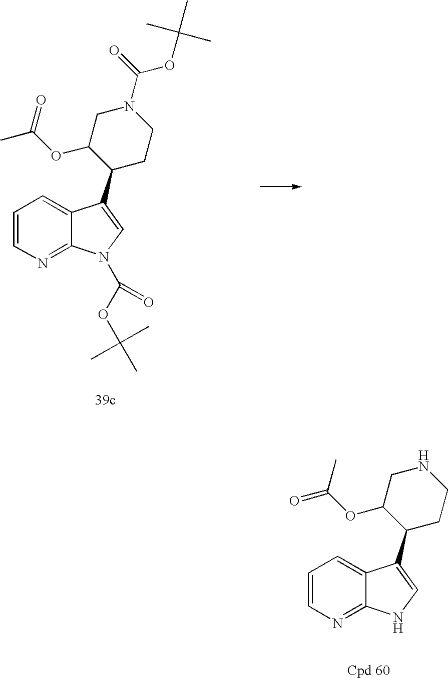

Compound 39c (0.04 g, 0.087 mmol) was dissolved in dry DCM (3 mL) and TFA (1.3 mL) was added at 0° C. The mixture was stirred at 0° C. for 1 hour and at room temperature for 18 hours. The reaction mixture was then partitioned with saturated aqueous sodium bicarbonate and DCM. The organic layer was washed with brine and dried with Na₂SO₄, then filtered and evaporated in vacuo to yield Compound 60 as a gum.

¹H NMR (400 MHz, CDCl₃) δ 10.24 (1H, br s), 8.31 (1H, dd, J=1.30 Hz & 4.70 Hz), 8.05 (1H, dd, J=1.39 Hz & 7.88 Hz), 7.17 (1H, s), 7.08 (1H, dd, J=4.75 Hz & 7.87 Hz), 4.96-4.90 (1H, m), 3.35 (1H, dd, J=4.40 Hz & 12.01 Hz), 3.19-3.04 (2H, m), 2.79-2.72 (1H, m), 2.67-2.62 (1H, m), 2.11-2.06 (1H, m), 2.02-1.83 (2H, m), 1.79 (3H, s); MS (ES⁺) m/z 260.1 (MH)⁺

### EXAMPLE 40

### acetic acid 1-acenaphthen-1-yl-4-(1 H-pyrrolo[2,3-b]pyridin-3-yl)-piperidin-3-yl ester (Cpd 39)

The title compound was prepared according to the procedure of Example 4, using 3-(1-tert-butoxycarbonyl-3-hydroxy-piperidin-4-yl)-pyrrolo[2,3-b]pyridine-1-carboxylic acid tert-butyl ester Compound 60 and 1-bromo-acenaphthene Compound 4b to yield Compound 39 as a diastereoisomeric mixture in the form of a gum.

¹H NMR (400 MHz, CDCl₃) δ 9.45 (2H, br s), 8.28 (2H, br s), 8.00 (2H, d, J=7.91 Hz), 7.22-7.69 (2H, m), 7.63 (2H, d, J=8.2 Hz), 7.55-7.51 (4H, m), 7.49-7.43 (2H, m), 7.30 (2H, d, J=6.77 Hz), 7.14 (2H, br s), 7.05 7.08 (2H, dd, J=4.70 Hz & 7.80 Hz), 5.16-5.06 (2H, m), 5.01-4.98 (2H, m), 3.46-3.43 (2H, m), 3.19-3.15 (1H, m), 3.01-2.98 (1H, m), 2.95-2.88 (2H, m), 2.76-2.73 (1H, m), 2.59-2.53 (1H, m), 2.43 (1H, t, J=9.90Hz), 2.36-2.30 (1H, m), 2.24 (1H, t, J=10.10 Hz), 2.09-1.96 4H, m), 1.72 (3H, s), 1.38 (3H, s); MS (ES⁺) m/z 412.1 (MH)⁺

### EXAMPLE 41

### 1-acenaphthen-1-yl-4-(1 H-pyrrolo[2,3-b]pyridin-3-yl)-piperidin-3-ol (Cpd 40)

Acetic acid 1-acenaphthen-1-yl-4-(1 H-pyrrolo [2,3-b]pyridin-3-yl)-piperidin-3-yl ester Compound 39 (0.047 g, 0.114 mmol) was dissolved in dry methanol (3 mL). A solution of sodium methoxide (230 μL of a 0.5M solution of MeONa in MeOH) was added at room temperature and the mixture was stirred at room temperature for 18 hours, then concentrated in vacuo and partitioned with aqueous 1M NaOH and DCM. The organic layer was washed with brine and dried with Na₂SO₄, then filtered and evaporated in vacuo to yield Compound 40 as a diastereoisomeric mixture in the form of a gum.

¹H NMR (400 MHz, CD₃OD) δ 8.14-8.12 (4H, m), 7.71 (2H, d, J=7.98 Hz), 7.66-7.56 (4H, m), 7.55-7.51 (2H, m), 7.48-7.44 (2H, m), 7.32 (2H, d, J=6.80 Hz), 7.24 (2H, s), 7.05 (2H, dd, J=5.1 Hz & 7.40 Hz), 5.04-4.99 (2H, m), 3.96-3.90 (1H, m), 3.87-3.81 (1H, m), 3.55-3.41 (4H, m), 3.14-3.10 (1H, m), 2.98-2.96 (1H, m), 2.87-2.83 (1H, m), 2.74-2.54 (1H, m), 2.41-2.30 (2H, m), 2.20-2.01 (2H, m), 1.96-1.88 (3H, m); MS (ES⁺) m/z 370.2 (MH)⁺

### EXAMPLE 42

### 3-(1-acenaphthen-1-yl-1,2,3,6-tetrahydro-pyridin-4-yl)-5-isocyano-1H-indole (Cpd 42)

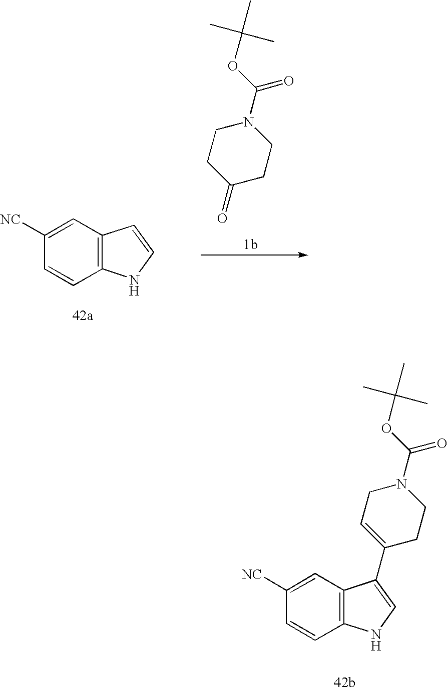

Using the procedure of Example 1 and 5-isocyano-1H-indole Compound 42a yielded 4-(5-cyano-1H-indol-3-yl)-3,6-dihydro-2H-pyridine-1-carboxylic acid tert-butyl ester Compound 42b. MS m/z 324 (M⁺H).

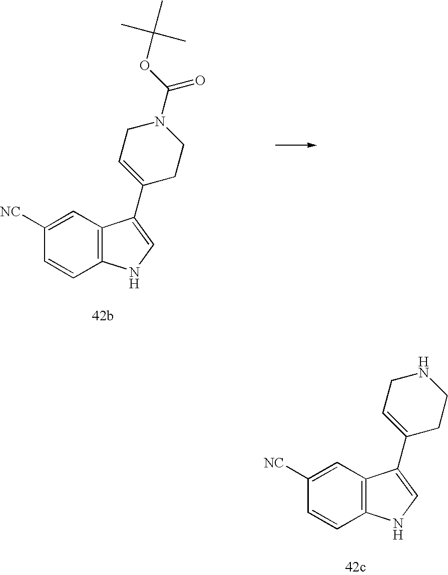

Using the procedure of Example 1 and Compound 42b in place of 4-(5-fluoro-1H-indol-3-yl)-piperidine-1-carboxylic acid tert-butyl ester Compound 1d yielded 3-(1,2,3,6-tetrahydro-pyridin-4-yl)-1H-indole-5-carbonitrile Compound 42c. MS m/z 224 (M⁺H).

The title compound was prepared according to the procedure of Example 4, using Compound 42c and 1-bromo-acenaphthene Compound 4b to yield Compound 42.

¹H NMR (300 MHz, CDCl₃) δ 8.38 (2H, br s), 8.22 (1H, s), 7.79-1.64 (2H, m), 7.58-7.46 (4H, m), 7.42-7.41 (1H, m), 7.33-7.31 (1H, m), 7.23 (1H, d, J=2.50 Hz), 6.16 (1H, br s), 5.11 (1H, t, J=5.60 Hz), 3.86-3.78 (1H, m), 3.50 (1H, d, J=5.60 Hz), 3.35-3.23 (2H, m), 2.87-2.77 (1H, m), 2.73-2.66 (1H, m), 2.57 (2H, br s); MS (ES⁺) m/z 376.2 (MH)⁺

### EXAMPLE 43

### (1S)-3-(1-acenaphthen-1-yl-1,2,3,6-tetrahydro-pyridin-4-yl)-5-isocyano-1-oxiranylmethyl-1H-indole (Cpd 43)

The title compound was prepared according to the procedure of Example 17, using 3-(1-acenaphthen-1-yl-1,2,3,6-tetrahydro-pyridin-4-yl)-5-isocyano-1H-indole Compound 42 to yield Compound 43 as an oil.

¹H NMR (300 MHz, CDCl₃) δ 8.20 (1H, s), 8.01 (1H,s), 7.73-7.70 (1H, m), 7.65 (1H, d, J=8.20 Hz), 7.57-7.38 (4H, m), 7.32 (1H, d, J=6.80 Hz), 7.16 (1H, s), 6.14 (1H, br s), 5.10 (1H, t, J=5.60 Hz), 4.48 (1H, dd, J=2.50 Hz & 15.30 Hz), 4.08 (1H, ddd, J=1.60 Hz, 5.70 Hz & 15.3 Hz), 3.49 (2H, d, J=5.60 Hz), 3.33-3.24 (2H, m), 2.84-2.64 (4H, m), 2.56-2.54 (2H, m), 2.46-2.43 (1H, m); MS (ES⁺) m/z 432.3 (MH)⁺

### EXAMPLE 44

### (1R)-1-[3-(1-acenaphthen-1-yl-1,2,3,6-tetrahydro-pyridin-4-yl)-5-isocyano-indol-1-yl]-3-amino-propan-2-ol (Cpd 44)

The title compound was prepared according to the procedure of Example 23, using (1S)-3-(1-acenaphthen-1-yl-1,2,3,6-tetrahydro-pyridin-4-yl)-5-isocyano-1-oxiranylmethyl-1H-indole Compound 43 and 2.0M ammonium hydroxide in EtOH yielded Compound 44 as an oil.

¹H NMR (300 MHz, CDCl₃) δ 8.19 (1H, br s), 7.72 (1H, d, J=7.20 Hz), 7.65 (1H, d, J=8.20 Hz), 7.55-7.46 (3H, m), 7.41-7.37 (2H, m), 7.32 (1H, d, J=6.60 Hz), 7.21 (1H, s), 6.12 (1H, br s), 5.09-5.08 (1H, m), 4.19 (1H, dd, J=4.20 Hz & 15.00 Hz), 4.10 (1H, dd, J=6.05 Hz & 14.65 Hz), 3.88-3.84 (1H, m), 3.55-3.48 (3H, m), 3.32-3.27 (1H, m), 2.90-2.85 (1H, m), 2.78-.272 (1H, m), 2.70-2.67 (1H, m), 2.56-2.49 (3H, m), 2.01 (2H, br s); MS (ES⁺) m/z 449.2 (MH)⁺

### EXAMPLE 45

### (1R)-1-[3-(1-acenaphthen-1-yl-1,2,3,6-tetrahydro-pyridin-4-yl)-5-isocyano-indol-1-yl]-3-methylamino-propan-2-ol (Cpd 45)

The title compound was prepared according to the procedure of Example 24, using (1S)-3-(1-acenaphthen-1-yl-1,2,3,6-tetrahydro-pyridin-4-yl)-5-isocyano-1-oxiranylmethyl-1H-indole Compound 43 and 2.0M methylamine in MeOH to yield Compound 45 as an oil.

¹H NMR (300 MHz, CDCl₃) δ 8.20 (1H, br s), 7.72 (1H, d, J=7.90 Hz), 7.65 (1H, d, J=8.20 Hz), 7.60-7.46 (3H, m), 7.44-7.38 (2H, m), 7.32 (1H, d, J=6.80 Hz), 7.21 (1H, s), 6.13 (1H, br s), 5.10 (1H, t, J=5.70 Hz), 4.23-4.17 (1H, m), 4.14-4.05 (1H, m), 4.00-3.93 (1H, m), 3.56-3.48 (3H, m), 3.33-3.27 (1H, m), 2.81-2.76 (1H, m), 2.73-2.64 (2H, m), 2.56 (2H, br s), 2.46-2.41 (1H, m), 2.39 (3H, s); MS (ES⁺) m/z 463.2 (MH)⁺

### EXAMPLE 46

### (1R)-1-[3-(1-acenaphthen-1-yl-1,2,3,6-tetrahydro-pyridin-4-yl)-5-isocyano-indol-1-yl]-3-dimethylamino-propan-2-ol (Cpd 46)

The title compound was prepared according to the procedure of Example 25, using (1S)-3-(1-acenaphthen-1-yl-1,2,3,6-tetrahydro-pyridin-4-yl)-5-isocyano-1-oxiranylmethyl-1H-indole Compound 43 and 2.0M dimethylamine in MeOH to yield Compound 46 as an oil.

¹H NMR (300 MHz, CDCl₃) δ 8.20 (1H, br s), 7.72 (1H, d, J=7.20 Hz), 7.65 (1H, d, J=8.20 Hz), 7.58-7.45 (3H, m), 7.44-7.38 (2H, m), 7.32 (1H, d, J=6.80 Hz), 7.23 (1H, s), 6.14 (1H, br s), 5.10 (1H, t, J=5.50 Hz), 4.18 (1H, dd, J=3.70 Hz & 14.50 Hz), 4.08 (1H, dd, J=5.80 Hz & 14.50 Hz), 4.00-3.92 (1H, m), 3.56-3.48 (3H, m), 3.34-3.28 (1H, m), 2.84-2.76 (1H, m), 2.73-2.65 (1H, m), 2.62-2.49 (2H, m), 2.24 (6H, s), 2.22-2.20 (2H, m); MS (ES⁺) m/z 477.1 (MH)⁺

### EXAMPLE 47

### 6-fluoro-3-(1,2,3,6-tetrahydro-pyridin-4-yl)-1H-indole (Cpd 48)

The title compound was prepared according to the procedure of Example 1, using 4-(6-fluoro-1H-indol-3-yl)-3,6-dihydro-2H-pyridine-1-carboxylic acid tert-butyl ester Compound 47 in place of 5-fluoro-3-piperidin-4-yl-1H-indole Compound 1e to yield Compound 48 as a solid. MS m/z 217 (MH)⁺

### EXAMPLE 48

### 3-(1-acenaphthen-1-yl-1,2,3,6-tetrahydro-pyridin-4-yl)-6-fluoro-1H-indole (Cpd 49)

The title compound was prepared according to the procedure of Example 4, using 1-bromo-acenaphthene Compound 4b and 4-(6-fluoro-1H-indol-3-yl)-3,6-dihydro-2H-pyridine-1-carboxylic acid tert-butyl ester Compound 47 to yield Compound 49 as an oil.

¹H NMR (300 MHz, DMSO) δ 7.78-7.73 (2H, m), 7.67 (1H, d, J=8.20 Hz), 7.57-7.47 (3H, m), 7.35-7.33 (2H, m), 7.13 (1H, dd, J=2.30 Hz & 9.90 Hz), 6.89-6.82 (1H, m), 6.10 (1H, br s), 5.06-5.03 (1H, m), 4.04 (2H, br s), 3.41-3.39 (2H, m), 3.33-3.31 (2H, m), 3.10-3.04 (1H, m), 2.79-2.73 (1H, m), 2.61-2.56 (1H, m); MS (ES⁺) m/z 369.1 (MH)⁺

### EXAMPLE 49

### 3-(1-acenaphthen-1-yl-1,2,3,6-tetrahydro-pyridin-4-yl)-1-ethyl-6-fluoro-1H-indole (Cpd 50)

The title compound was prepared according to the procedure of Example 36, using ethyl iodide and 3-(1-acenaphthen-1-yl-1,2,3,6-tetrahydro-pyridin-4-yl)-6-fluoro-1H-indole Compound 49 to yield Compound 50 as an oil. MS (ES⁺) m/z 397.1 (MH)⁺

### EXAMPLE 50

### 3-(1-benzyl-1,2,3,6-tetrahydro-pyridin-4-yl)-5-chloro-1H-indole (Cpd 51)

Using the procedure of Example 1 and 5-chloro-1H-indole Compound 29a yielded 4-(5-chloro-1H-indol-3-yl)-3,6-dihydro-2H-pyridine-1-carboxylic acid tert-butyl ester Compound 50a. MS m/z 333 (MH)⁺.

Using the procedure of Example 1 and Compound 50a in place of 4-(5-fluoro-1H-indol-3-yl)-piperidine-1-carboxylic acid tert-butyl ester Compound 1d yielded 5-chloro-3-(1,2,3,6-tetrahydro-pyridin-4-yl)-1H-indole Compound 50b. MS m/z 233 (MH)⁺.

The title compound was prepared according to the procedure of Example 1, using Compound 50b and benzaldehyde Compound 50c to yield Compound 51.

¹H NMR (400 MHz, CDCl₃) δ 8.17 (1H, br s), 7.83 (1H, d, J=2.0 Hz), 7.41-7.39 (m, 2H), 7.36-7.32 (2H, m), 7.29-7.24 (2H, m), 7.14 (2H, dd, J=2.10 Hz & 8.10 Hz), 6.14-6.12 (1H, m), 3.66 (2H, s), 3.24-3.22 (m, 2H), 2.74-2.72 (2H, m), 2.57-2.55 (2H, m); MS (ES⁺) m/z 323.0 (MH)⁺

### EXAMPLE 51

### 3-(1-cyclohexylmethyl-piperidin-4-yl)-1H-pyrrolo [2,3-b]pyridine (Cpd 52)

The title compound was prepared according to the procedure of Example 1, using cyclohexanecarboxylic acid to yield Compound 52 as a solid.

¹H NMR (400 MHz, CDCl₃) δ 9.95 (1H, br s), 8.29 (1H, dd, J=1.43 Hz & 4.75 Hz), 7.97 (1H, dd, J=1.40 Hz & 7.85 Hz), 7.09 (1H, d, J=1.20 Hz), 7.04 (1H, dd, J=4.75 Hz & 7.85 Hz), 3.76-3.73 (1H, m), 3.00-2.97 (2H, m), 2.78-2.75 (1H, m), 2.16 (1H, d, J=7.05 Hz), 2.07-1.97 (4H, m), 1.88-1.65 (6H, m), 1.53-1.51 (1H, m), 1.26-1.15 (3H, m), 0.94-0.88 (2H, m); MS (ES⁺) m/z 298.1 (MH)⁺

### EXAMPLE 52

### 3-(1-hexyl-piperidin-4-yl)-1H-pyrrolo[2,3-b]pyridine (Cpd 53)

The title compound was prepared according to the procedure of Example 1, using hexanal to yield Compound 53 as a solid.

¹H NMR (400 MHz, CDCl₃) δ 10.17 (1H, br s), 8.29 (1H, dd, J=1.44 Hz & 4.75 Hz), 7.97 (1H, dd, J=1.40 Hz & 7.85 Hz), 7.10 (1H, d, J=1.40 Hz), 7.04 (1H, dd, J=4.75 Hz & 7.85 Hz), 3.08-3.06 (2H, m), 2.81-2.77 (1H, m), 2.39-2.34 (2H, m), 2.16-2.00 (4H, m), 1.90-1.83 (2H,m), 1.56-1.52 (2H, m), 1.33-1.25 (6H, m), 0.91-0.87 (3H, m); MS (ES⁺) m/z 286.0 (MH)⁺

### EXAMPLE 53

### 3-(1-cyclopropylmethyl-piperidin-4-yl)-1H-pyrrolo [2,3-b]pyridine (Cpd 54)

The title compound was prepared according to the procedure of Example 1, using cyclopropanecarbaldehyde to yield Compound 54 as a solid.

¹H NMR (400 MHz, CDCl₃) δ 10.23 (1H, br s), 8.30 (1H, dd, J=1.44 Hz & 4.75 Hz), 7.97 (1H, dd, J=1.40 Hz & 7.85 Hz), 7.12 (1H, s), 7.05 (1H, dd, J=4.75 Hz & 7.85 Hz), 3.23-3.20 (2H, m), 2.83-2.76 (1H, m), 2.32 (2H, d, J=6.55 Hz), 2.22-2.11 (2H, m), 2.05-2.02 (2H, m), 1.93-1.83 (2H, m), 0.95-0.89 (1H, m), 0.56-0.51 (2H, m), 0.15-0.11 (2H, m); MS (ES⁺) m/z 256.0 (MH)⁺

### EXAMPLE 54

### 3-[1-(4-phenoxy-benzyl)-piperidin-4-yl]-1H-pyrrolo[2,3-b]pyridine (Cpd 55)

The title compound was prepared according to the procedure of Example 1, using 4-phenoxy-benzaldehyde Compound 54a to yield Compound 55 as a solid.

¹H NMR (400 MHz, CDCl₃) δ 8.88 (1H, br s), 8.28 (1H, dd, J=1.15 Hz & 4.60 Hz), 7.96 (1H, d, J=7.90 Hz), 7.35-7.30 (4H, m), 7.11-6.96 (7H, m), 3.55 (2H, s), 3.04-3.01 (2H, m), 2.83-2.77 (1H, m), 2.18-2.13 (2H, m), 2.02-1.99 (2H, m), 1.89-1.80 (2H, m); MS (ES⁺) m/z 384.0 (MH)⁺

### EXAMPLE 55

### 2-benzo[1,3]dioxol-5-yl-1-[4-(1H-pyrrolo[2,3-b]pyridin-3-yl)-piperidin-1-yl]-ethanone (Cpd 56)

Benzo[1,3]dioxol-5-yl-acetic acid (0.0175 g, 0.097 mmol) was suspended in dry DCM (2 mL) and dry DMF (0.1 mL). 1-(3-dimethylaminopropyl)-3-ethylcarbodiimide HCl salt (0.0205 g, 0.107 mmol) and HOBT (0.0171 g, 0.126 mmol) were added and the mixture was stirred under a nitrogen atmosphere at room temperature for 30 minutes. A solution of 3-piperidin-4-yl-1H-pyrrolo[2,3-b]pyridine Compound 30 (0.029 g, 0.097 mmol) in dry DCM (1 mL) was added under a nitrogen atmosphere and the mixture was stirred under a nitrogen atmosphere at room temperature for 4 days. The reaction mixture was partitioned with aqueous NaHCO₃ and DCM. The organic layer was washed with aqueous 0.5 N HCl and brine and dried with Na₂SO₄, then filtered and evaporated in vacuo to yield a crude oil. The crude oil was purified via flash chromatography (5% ammonia 2.0 M in methanol/DCM) to yield Compound 56 as a solid.

¹H NMR (400 MHz, CDCl₃) δ 9.88 (1H, br s), 8.30 (1H, d, J=3.38 Hz), 7.88 (1H, dd, J=1.20 Hz & 7.88 Hz), 7.07-7.04 (2H, m), 6.81 (1H, d, J=1.45 Hz), 6.77-6.70 (2H, m), 5.93 (2H, s), 4.80-4.77 (2H, m), 4.01-3.97 (2H, m), 3.70 (2H, s), 3.20-3.13 (1H, m), 3.05-2.97 (1H, m), 2.78-2.72 (2H, m), 2.06-1.95 (2H, m), 1.75-1.64 (1H, m), 1.52-1.41 (1H, m); MS (ES⁺) m/z 363.9 (MH)⁺

### EXAMPLE 56

### 3-(1-benzyl-piperidin-4-yl)-5-chloro-1H-indole (Cpd 57)

The title compound was prepared according to the procedure of Example 1, using 5-chloro-3-piperidin-4-yl-1H-indole Compound 29 and benzaldehyde Compound 50c to yield Compound 57 as a solid.

¹H NMR (400 MHz, CDCl₃) δ 7.99 (1H, br s), 7.59 (1H, d, J=1.87 Hz), 7.37-7.31 (4H, m), 7.28-7.24 (2H, m), 7.12 (1H, dd, J=1.97 Hz & 8.60 Hz), 6.97 (1H, d, J=2.20 Hz), 3.57 (2H, s), 3.03-3.00 (2H, m), 2.80-2.72 (1H, m), 2.18-2.12 (2H, m), 2.01-198 (2H, m), 1.84-1.74 (2H, m); MS (ES⁺) m/z 324.9 (MH)⁺

### EXAMPLE 57

### 7-chloro-3-(1,2,3,6-tetrahydro-pyridin-4-yl)-1H-indole (Cpd 58)

Using the procedure of Example 1 and 7-chloro-1H-indole Compound 29a yielded 4-(7-chloro-1H-indol-3-yl)-3,6-dihydro-2H-pyridine-1-carboxylic acid tert-butyl ester Compound 57a. MS m/z 333 (MH)⁺.

The title compound was prepared according to the procedure of Example 1, using Compound 57a in place of 4-(5-fluoro-1H-indol-3-yl)-piperidine-1-carboxylic acid tert-butyl ester Compound 1d to yield Compound 58.

¹H NMR (400 MHz, DMSO) δ 11.12 (1H, br s), 7.54 (1H, d, J=7.86 Hz), 7.19-7.12 (2H, m), 6.98-6.94 (1H, m), 3.34-3.31 (1H, br s), 3.02-2.99 (2H, m), 2.85-2.79 (1H, m), 2.66-2.59 (2H, m), 1.86-1.81 (2H, m), 1.59-1.49 (2H, m); MS (ES) m/z 234.9 (MH)⁺

### EXAMPLE 58

### (4-tert-butyl-cyclohexyl)-[4-(1H-pyrrolo[2,3-b]pyridin-3-yl)-piperidin-1-yl]-methanone (Cpd 59)

The title compound was prepared according to the procedure of Example 55, using 4-tert-butyl-cyclohexanecarboxylic acid Compound 58a to yield Compound 59 as a gum.

¹H NMR (400 MHz, CDCl₃) δ 9.69 (1H, br s), 8.31 (1H, d, J=3.80 Hz), 7.94 (1H, dd, J=1.00 Hz & 7.90 Hz), 7.09-7.06 (2H, m), 4.81-4.78 (1H, m), 4.07-4.04 (1H, m), 3.23-3.17 (1H, m), 3.09-3.02 (1H, m), 2.74-2.68 (1H, m), 2.48-2.42 (1H, m), 2.14-2.05 (2H, m), 1.87-1.54 (11H, m), 0.86 (9H, s); MS (ES⁺) m/z 368.1 (MH)⁺

### BIOLOGICAL EXAMPLES

The ability of the compounds and their use in treating, preventing or ameliorating an ORL-1 receptor mediated disorder and condition is determined using the following procedures.

### EXAMPLE 1

Production of Cells Expressing the ORL-1, Delta, Kappa or Mu Receptor

HEK293 cells are transfected with the nociceptin receptor (ORL-1, human mRNA GenBank #AF348323) or any of the opioid receptor subtypes: delta (δ, human mRNA Genbank #U07882) kappa (κ, human mRNA Genbank #U17298) and mu (μ, human mRNA Genbank #L29301). The vector used is pCi-neo (G418 selection). The transfections are performed with LipofectAMINE 2000 (Life Technologies Cat. #11668-019) using the following procedure.

The day before transfection, a 24 well plate is inoculated with 2×10⁵ cells per well in 0.5 mL of normal growth medium (MEM+EBSS+NEAA+10% BCS). Two wells are prepared for each receptor, one each of which is a corresponding control.

For each well transfected, DNA (0.8 μg) is diluted (50 μL total volume) using OPTI-MEM I Reduced Serum Medium (Life Technologies Cat. #51985-034).

For each well transfected, LipofectAMINE 2000 (LF2000) (2 μL) is added to the diluted DNA medium and the mixture is incubated for 5 minutes at room temperature. The diluted DNA and LF2000 are combined and incubated at room temperature for 20 minutes. The growth medium is aspirated from each well and replaced with OPTI-MEM I (1 mL). The DNA-LF2000 complexes (100 μL) are added to each well and mixed with gentle swirling. The plate is incubated at 37° C., under a 5% CO₂ blanket for 5 hours. The OPTI-MEM I medium is aspirated from each transfected well and replaced with growth medium (1 mL). The plate is returned to the incubator for 24 hours. The wells are trypsinized and the cells are added to 100 mm tissue culture dishes (2 dishes per well). The dishes are incubated for 24 hours, then the medium is aspirated from each dish and replaced with growth medium containing 400μg/ml Geneticin (G418) selective antibiotic. The plates are read every 3-4 days.

Distinct colonies appear in approximately 3 weeks. One week later, 48 out of approximately 100 colonies per dish are subcultured to each well of two 24 well plates containing selective medium (1 mL per well).

Confluent wells are expanded to 6 well plates, then T25 flasks and T75 flasks. Cell lines showing poor growth patterns are eliminated. Membranes are prepared from each cell line and receptor activity is determined by a receptor binding assay.

Assay to Measure Affinity of Radiolabeled ORL-1 Ligands

The nociceptin receptor binding assay measures the binding of ¹²⁵I-Tyr¹⁴-nociceptin (2200 Ci/mmol, New England Nuclear) to human nociceptin receptor (ORL-1) on HEK293 cell membranes.

HEK293 cell membrane (prepared as described in Pulito, V L, et al., J. Pharmacol. Exp. Ther. 2000, 294, 224-229, with the exception that the buffer used is a mixture of 50 mM Tris-HCl pH 7.8, 5 mM MgCl₂ and 1 mM EGTA) is added to PEI treated WGA FlashPlates (New England Nuclear) at 1 μg/well in a binding buffer (50 mM Tris-HCl pH 7.8, 5 mM MgCl₂ and 1 mM EGTA). ¹²⁵I-Tyr¹⁴-nociceptin is added at a final concentration of 0.5 nM and the volume adjusted to 50 μL with binding buffer. The plate is incubated for two hours at room temperature, the reactions are aspirated and the wells washed two times with binding buffer (200 μL) and then filled with binding buffer (200 μL). The plates are then sealed and counted on a Packard Top Count to determine radioactivity bound to the membranes.

For each test compound, the total binding (% Inh) was measured at several concentrations and the IC₅₀ (the concentration at which 50% of the binding is inhibited) was determined using Graphpad Prizm software.

The ORL-1 receptor binding activity results for representative compounds of the present invention is shown in Table 1.

TABLE 1
ORL-1 IC₅₀ (nM)

| Cpd | IC₅₀ |
|---|---|
| 1 | 806 |
| 2 | 1779 |
| 3 | 291.2 |
| 4 | 18.3 |
| 5 | 5000 |
| 6 | 10.7 |
| 7 | 5000 |
| 8 | 5000 |
| 9 | 5000 |
| 10 | 5000 |
| 11 | 5000 |
| 12 | 712 |
| 13 | 5000 |
| 14 | 5000 |
| 15 | 658.8 |
| 16 | 57.4 |
| 17 | 210.9 |
| 18 | 138.3 |
| 19 | 971.1 |
| 20 | 5000 |
| 21 | 5000 |
| 22 | 52.9 |
| 23 | 62.6 |
| 24 | 103.1 |
| 25 | 100.7 |
| 31 | 1956 |
| 32 | 557.1 |
| 33 | 820.6 |
| 34 | 4.3 |
| 35 | 81.3 |
| 36 | 81.7 |
| 37 | 550.6 |
| 38 | 94.5 |
| 39 | 5000 |
| 40 | 2.5 |
| 42 | 5000 |
| 43 | 5000 |
| 44 | 1647 |
| 45 | 1818 |
| 46 | 5000 |
| 47 | 5000 |
| 48 | 5000 |
| 49 | 164.3 |
| 50 | 5000 |
| 51 | 5000 |
| 52 | 3504 |
| 53 | 5000 |
| 54 | 5000 |
| 55 | 5000 |
| 56 | 5000 |
| 57 | 1142 |
| 58 | 5000 |
| 59 | 5000 |

### EXAMPLE 2

Filtration Binding Assay: Mu, Kappa and Delta Opioid Receptors

Prepared similarly to the procedure of Example 1, with appropriate selection and substitution of cell membrane and radiolabeled ligands, the following assay is used to measure the binding of representative test compounds to the ORL-1, delta, kappa and mu opioid receptors.

For determining the binding of a test compound to the delta (δ) opioid receptor, the cell membrane and ligand used, respectively, are the 2D4 cell line membrane (5 μg/well) and a 1:1000 ratio of the DPDPE-H³ ligand.

For determining the binding of a test compound to the mu (μ) opioid receptor, the cell membrane and ligand used, respectively, are the 1D4 cell line membrane (10 μg/well) and a 1:1000 ratio of the Damgo-H³ ligand.

For determining the binding of a test compound to the kappa (κ) opioid receptor, the cell membrane and ligand used, respectively, are the 2C2 cell line membrane (5 μg/well) and a 1:1000 ratio of the U69593-H³ ligand.

Both membrane and ligand are diluted such that a 25 μL addition each is delivered per well. Both membrane and ligand are diluted in ORL-1 buffer (1×) (a mixture of 50 mM Tris-HCl, pH 7.4, 5 mM MgCl₂ and 1 mM EGTA). Each test compound is diluted to a concentration in the range of from 100 μM to 10 pM with 100% DMSO.

The diluted test compound (1 μL), cell membrane (25 μL) and labeled ligand (25 μL) for the mu, delta, kappa or ORL-1 opioid receptor, as desired, are added to each well of a 96 well plate.

The plate is incubated on a rotating shaker for 2 hours at room temperature, then filtered over GF/C Filterplates, prewetted in 0.03% polyethleneimine, in a Filtermate 196 apparatus (Packard). The plate is then washed 6 times with ORL-1 buffer in the filtration apparatus and dried in a vacuum oven for 1 hour at a temperature of 50° C.

Microscint 20 (Packard) (25 μL) is added to each well to solubilize bound radioactivity, then each well is counted in a Packard TopCount for 1 minute/well using counting parameters optimized for the particular radioligand/opioid receptor being tested. The amount (in percent) of radioactive ligand bound in each reaction is calculated relative to a control using DMSO for maximum binding (no inhibition). The curves are fitted and the IC₅₀ is determined using Graphpad Prizm software (v3.0).

The results for representative compounds of the present invention tested for binding to the mu, kappa and delta opioid receptors are listed in Table 2.

TABLE 2
Opioid Receptor IC₅₀ (nM)

| Cpd | Mu | Kappa | Delta |
|---|---|---|---|
| 1 | 501.7 | 820.4 | 5000 |
| 2 | 4707 | 5000 | 5000 |
| 3 | 1081 | 182 | 5000 |
| 4 | 506.6 | 1890 | 5000 |
| 5 | 5000 | 5000 | 5000 |
| 6 | 260.7 | 687.16 | 2746.3 |
| 7 | 5000 | 5000 | 5000 |
| 8 | 5000 | 5000 | 5000 |
| 9 | 5000 | 5000 | 5000 |
| 10 | 888.2 | 754.9 | 5000 |
| 11 | 5000 | 1569 | 5000 |
| 12 | 1359 | 332.5 | 4183 |
| 13 | 443.3 | 4077 | 5000 |
| 14 | 5000 | 5000 | 5000 |
| 15 | 436.2 | 1168 | 5000 |
| 16 | 263.8 | 2598 | 5000 |
| 17 | 671.2 | 5000 | 5000 |
| 18 | 5000 | 5000 | 5000 |
| 19 | 2698 | 1895 | 5000 |
| 20 | 5000 | 5000 | 5000 |
| 21 | 5000 | 5000 | 5000 |
| 22 | 902.9 | 5000 | 5000 |
| 23 | 765.4 | 5000 | 5000 |
| 24 | 805.1 | 5000 | 5000 |
| 25 | 473.3 | 5000 | 5000 |
| 31 | 5000 | 5000 | 5000 |
| 32 | 1996 | 3885 | 5000 |
| 33 | 344.5 | 710.7 | 5000 |
| 34 | 389.66 | 2779.25 | 5000 |
| 35 | 1195 | 840.2 | 5000 |
| 36 | 905 | 5000 | 5000 |
| 37 | 1025 | 5000 | 5000 |
| 38 | 530.4 | 5000 | 5000 |
| 39 | 5000 | 5000 | 5000 |
| 40 | 169 | 4300 | 5000 |
| 42 | 5000 | 5000 | 5000 |
| 43 | 1144 | 5000 | 5000 |
| 44 | 4143 | 5000 | 5000 |
| 45 | 2570 | 5000 | 5000 |
| 46 | 4136 | 5000 | 5000 |
| 47 | 5000 | 5000 | 5000 |
| 48 | 5000 | 5000 | 5000 |
| 49 | 5000 | 5000 | 5000 |
| 50 | 2614 | 5000 | 5000 |
| 51 | 507.4 | 1014 | 5000 |
| 52 | 5000 | 5000 | 5000 |
| 53 | 5000 | 5000 | 5000 |
| 54 | 5000 | 5000 | 5000 |
| 55 | 339.1 | 5000 | 5000 |
| 56 | 5000 | 5000 | 5000 |
| 57 | 236.9 | 328.7 | 5000 |
| 58 | 255.5 | 5000 | 5000 |
| 59 | 5000 | 5000 | 5000 |

### EXAMPLE 3

ORL-1 Calcium Flux Functional Assay

A calcium flux assay, a HEK-293 cell line that overexpresses the ORL-1 receptor and the Gqi5 G protein (Molecular Devices) are used to assay the functional agonist or antagonist activity of a test compound.

HEK-293 cells are plated two days prior to assay. At the time of the assay, the cells in medium (50 μL) are incubated with dye (Molecular Devices) (50 μL) for 1 hour at 37° C. A test compound (100 μL) diluted in Hank's Buffered Salt Solution (HBSS) at 2-fold the indicated final concentration is added. Readings are taken at 1 second intervals for 1 minute, then 3 second intervals for 1 minute using FLIPR384 (Molecular Devices). Nociceptin (Neosystems, SA) (50 μL) at 5-fold the indicated final concentration is added and readings are taken at the same intervals as those taken for the test compound.

The data is processed using Microsoft Excel 6.0 and the EC₅₀ values are determined using GraphPad Prism 3.0. For a functional agonist compound, the EC₅₀ is determined from the initial calcium signal obtained after addition of the test compound. For a functional antagonist compound, the percent inhibition or IC₅₀ is determined from the signal resulting from the subsequent addition of the nociceptin peptide.

The results for representative compounds of the present invention tested are listed in Table 3.

TABLE 3
ORL-1 Functional Agonist EC₅₀ (μM)4

| Cpd | EC₅₀ |
|---|---|
| 4 | 27.2 |
| 6 | 54.6 |
| 16 | 100 |
| 34 | 4.2 |

### EXAMPLE 4

As a specific embodiment of an oral composition, Compound 34 (100 mg) is formulated with a suitable finely divided lactose to provide a total amount of from about 580 mg to about 590 mg, sufficient to fill a size O hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

What is claimed is:

1. A compound of formula (I) 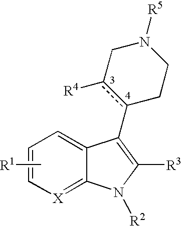
and forms thereof, wherein
the dashed line between position 3 and 4 in formula (I) represents a location for an optionally present double bond;
X is selected from the group consisting of CH and N;
R¹ is hydrogen or one, two, three or four substituents each selected from the group consisting of C₁₋₈alkyl, C₁₋₈alkoxy, amino, amino-C₁₋₈alkyl, halogen, C₁₋₈alkyl-halo, C₁₋₈alkoxy-halo, hydroxy, cyano and nitro;
R² is selected from the group consisting of hydrogen, C₃₋₁₄cycloalkyl and C₁₋₈alkyl; optionally substituted on C₁₋₈alkyl with one, two or three substituents each selected from the group consisting of C₁₋₈alkoxy, C₁₋₈acyl, oxy-C₁₋₈acyl, amino, amino-C₁₋₈alkyl, halogen, C₁₋₈alkyl-halo, hydroxy, carbonyl-C₁₋₈alkoxy, aryl, oxy-aryl, heterocyclyl, oxy-heterocyclyl, C₃₋₁₄cycloalkyl and oxy-C₃₋₁₄cycloalkyl;
R³ is selected from the group consisting of hydrogen and C₁₋₈alkyl;
R⁴ is one substituent when the double bond between position 3 and 4 in formula (I) is present or two substituents when the double bond between position 3 and 4 in formula (I) is not present, wherein each substituent is each selected from the group consisting of hydrogen, hydroxy and oxy-C₁₋₈acyl; and
R⁵ is selected from the group consisting of hydrogen, C₁₋₈alkyl, C₁₋₈acyl, carbonyl-C₁₋₈alkoxy, heterocyclyl, C₁₋₈acyl-heterocyclyl, C₃₋₁₄cycloalkyl, C₁₋₈alkyl-C₃₋₁₄cycloalkyl, carbonyl-C₃₋₁₄cycloalkyl, aryl and C₁₋₈alkyl-aryl,
wherein carbonyl-C₃₋₁₄cycloalkyl is optionally substituted on C₃₋₁₄cycloalkyl with one or two C₁₋₈alkyl substituents,
wherein C₁₋₈alkyl-aryl is optionally substituted on aryl with one, two or three substituents each selected from the group consisting of C₁₋₈alkyl, C₁₋₈alkoxy, C₁₋₈acyl, amino, amino-C₁₋₈alkyl, halogen, hydroxy, C₁₋₈alkyl-halo, C₁₋₈alkoxy-halo, aryl, oxy-aryl, heterocyclyl, oxy-heterocyclyl, C₃₋₁₄cycloalkyl and oxy-C₃₋₁₄cycloalkyl; and
wherein C₁₋₈alkyl is optionally substituted with one, two or three substituents each selected from the group consisting of C₁₋₈alkoxy, amino, amino-C₁₋₈alkyl, halogen, hydroxy and carbonyl-C₁₋₈alkoxy.

2. The compound of claim 1, wherein X is CH.

3. The compound of claim 1, wherein X is N.

4. The compound of claim 1, wherein R¹ is hydrogen or one, two, three or four substituents each selected from the group consisting of C₁₋₈alkyl, C₁₋₈alkoxy, amino, halogen, hydroxy, cyano and nitro.

5. The compound of claim 1, wherein R¹ is hydrogen or one, two, three or four substituents each selected from the group consisting of halogen and cyano.

6. The compound of claim 1, wherein R² is selected from the group consisting of hydrogen, C₃₋₁₄cycloalkyl and C₁₋₈alkyl; optionally substituted on C₁₋₈alkyl with one, two or three substituents each selected from the group consisting of amino, amino-C₁₋₈alkyl, hydroxy, carbonyl-C₁₋₈alkoxy, aryl, heterocyclyl and oxy-heterocyclyl.

7. The compound of claim 1, wherein R³ is hydrogen.

8. The compound of claim 1, wherein R³ is C₁₋₈alkyl.

9. The compound of claim 1, wherein R⁴ is one substituent, when the double bond between position 3 and 4 in formula (I) is present, selected from the group consisting of hydrogen, hydroxy and oxy-C₁₋₈acyl.

10. The compound of claim 1, wherein R⁴ is two substituents, when the double bond between position 3 and 4 in formula (I) is not present, each selected from the group consisting of hydrogen, hydroxy and oxy-C₁₋₈acyl.

11. The compound of claim 1, wherein R⁵is selected from the group consisting of hydrogen, C₁₋₈alkyl, carbonyl-C₁₋₈alkoxy, C₁₋₈acyl-heterocyclyl, C₃₋₁₄cycloalkyl, C₁₋₈alkyl-C₃₋₁₄cycloalkyl, carbonyl-C₃₋₁₄cycloalkyl and C₁₋₈alkyl-aryl,
wherein carbonyl-C₃₋₁₄cycloalkyl is optionally substituted on C₃₋₁₄cycloalkyl with one C₁₋₈alkyl substituent,
wherein C₁₋₈alkyl-aryl is optionally substituted on aryl with one substituent selected from the group consisting of C₁₋₈alkyl, C₁₋₈alkoxy, C₁₋₈acyl, amino, amino-C₁₋₈alkyl, halogen, hydroxy, C₁₋₈alkyl-halo, C₁₋₈alkoxy-halo, aryl, oxy-aryl, heterocyclyl, oxy-heterocyclyl, C₃₋₁₄cycloalkyl and oxy-C₃₋₁₄cycloalkyl, and
wherein C₁₋₈alkyl is optionally substituted with one substituent selected from the group consisting of C₁₋₈alkoxy, amino, amino-C₁₋₈alkyl, halogen, hydroxy and carbonyl-C₁₋₈alkoxy.

12. The compound of claim 1, wherein R⁵is selected from the group consisting of hydrogen, C₁₋₈alkyl, carbonyl-C₁₋₈alkoxy, C₁₋₈acyl-heterocyclyl, C₃₋₁₄cycloalkyl, C₁₋₈alkyl-C₃₋₁₄cycloalkyl, carbonyl-C₃₋₁₄cycloalkyl and C₁₋₈alkyl-aryl,
wherein carbonyl-C₃₋₁₄cycloalkyl is optionally substituted on C₃₋₁₄cycloalkyl with one C₁₋₈alkyl substituent, and
wherein C₁₋₈alkyl-aryl is optionally substituted on aryl with one substituent selected from the group consisting of C₁₋₈alkyl and oxy-aryl.

13. A compound of formula (Ia): 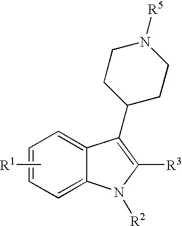
or a form thereof, wherein
R¹ is hydrogen or one, two, three or four substituents each selected from the group consisting of halogen and cyano;
R² is selected from the group consisting of hydrogen, C₃₋₁₄cycloalkyl and C₁₋₈alkyl; optionally substituted on C₁₋₈alkyl with one, two or three substituents each selected from the group consisting of amino, amino-C₁₋₈alkyl, hydroxy, carbonyl-C₁₋₈alkoxy, aryl, heterocyclyl and oxy-heterocyclyl;
R³ is selected from the group consisting of hydrogen and C₁₋₈alkyl; and
R⁵ is selected from the group consisting of hydrogen, C₁₋₈alkyl, carbonyl-C₁₋₈alkoxy, C₁₋₈acyl-heterocyclyl, C₃₋₁₄cycloalkyl, C₁₋₈alkyl-C₃₋₁₄cycloalkyl, carbonyl-C₃₋₁₄cycloalkyl and C₁₋₈alkyl-aryl,
wherein carbonyl-C₃₋₁₄cycloalkyl is optionally substituted on C₃₋₁₄cycloalkyl with one C₁₋₈alkyl substituent, and
wherein C₁₋₈alkyl-aryl is optionally substituted on aryl with one substituent selected from the group consisting of C₁₋₈alkyl and oxy-aryl.

14. A compound of formula (Ib): 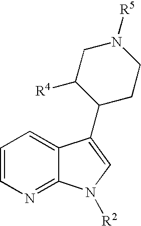
or a form thereof, wherein
R² is selected from the group consisting of hydrogen, C₃₋₁₄cycloalkyl and C₁₋₈alkyl; optionally substituted on C₁₋₈alkyl with one, two or three substituents each selected from the group consisting of amino, amino-C₁₋₈alkyl, hydroxy, carbonyl-C₁₋₈alkoxy, aryl, heterocyclyl and oxy-heterocyclyl;
R⁴ is two substituents each selected from the group consisting of hydrogen, hydroxy and oxy-C₁₋₈acyl; and
R⁵ is selected from the group consisting of hydrogen, C₁₋₈alkyl, carbonyl-C₁₋₈alkoxy, C₁₋₈acyl-heterocyclyl, C₃₋₁₄cycloalkyl, C₁₋₈alkyl-C₃₋₁₄cycloalkyl, carbonyl-C₃₋₁₄cycloalkyl and C₁₋₈alkyl-aryl,
wherein carbonyl-C₃₋₁₄cycloalkyl is optionally substituted on C₃₋₁₄cycloalkyl with one C₁₋₈alkyl substituent, and
wherein C₁₋₈alkyl-aryl is optionally substituted on aryl with one substituent selected from the group consisting of C₁₋₈alkyl and oxy-aryl.

15. A compound of formula (Ic): 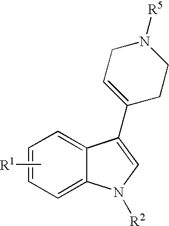
or a form thereof, wherein
R¹ is hydrogen or one, two, three or four substituents each selected from the group consisting of halogen and cyano;
R² is selected from the group consisting of hydrogen, C₃₋₁₄cycloalkyl and C₁₋₈alkyl; optionally substituted on C₁₋₈alkyl with one, two or three substituents each selected from the group consisting of amino, amino-C₁₋₈alkyl, hydroxy, carbonyl-C₁₋₈alkoxy, aryl, heterocyclyl and oxy-heterocyclyl; and
R⁵ is selected from the group consisting of hydrogen, C₁₋₈alkyl, carbonyl-C₁₋₈alkoxy, C₁₋₈acyl-heterocyclyl, C₃₋₁₄cycloalkyl, C₁₋₈alkyl-C₃₋₁₄cycloalkyl, carbonyl-C₃₋₁₄cycloalkyl and C₁₋₈alkyl-aryl,
wherein carbonyl-C₃₋₁₄cycloalkyl is optionally substituted on C₃₋₁₄cycloalkyl with one C₁₋₈alkyl substituent, and
wherein C₁₋₈alkyl-aryl is optionally substituted on aryl with one substituent selected from the group consisting of C₁₋₈alkyl and oxy-aryl.

16. The compound of any of claim 1 to 15, wherein the compound is an isolated form thereof.

17. The compound of claim 16, wherein the compound or a form thereof is an ORL-1 agonist or antagonist.

18. The compound of any of claim 1 to 16, wherein the form of said compound is a pharmaceutical composition or medicament comprising one or more of said compound.

19. A pharmaceutical composition comprising an effective amount of a compound of any of claim 1 to 16 and a pharmaceutically acceptable carrier.

20. A pharmaceutical composition made by mixing a compound of any of claim 1 to 16 and a pharmaceutically acceptable carrier.

21. A process for preparing a pharmaceutical composition comprising the step of admixing a compound of any of claim 1 to 16 and a pharmaceutically acceptable carrier.

22. The pharmaceutical composition of claim 19, wherein the effective amount of the compound is in a range of from about 0.001 mg/kg to about 300 mg/kg of body weight per day.

23. A method for using a compound of any of claim 1 to 16 for mediating ORL-1 receptor activity comprising contacting the receptor with one or more of the compounds.

24. A method for treating, preventing or ameliorating ORL-1 receptor mediated disorders and conditions in a subject in need thereof comprising administering to the subject an effective amount of a compound of any of claim 1 to 16.

25. The method of claim 24, wherein the disorder and condition is selected from the group consisting of anxiety, depression, panic, mania, dementia, bipolar disorder, substance abuse, neuropathic pain, acute pain, chronic pain migraine, asthma, cough, psychosis, schizophrenia, epilepsy, hypertension, obesity, eating disorders, cravings, diabetes, cardiac arrhythmia, irritable bowel syndrome, Crohn's disease, urinary incontinence, adrenal disorders, attention deficit disorder, attention deficit hyperactivity disorder and Alzheimer's disease.

26. The method of claim 24, wherein the method further comprises administering to the subject an effective amount of the compound, whereby the subject's cognition or memory is improved or mood is stabilized.

27. The method of any of claim 24 to 26, wherein the effective amount of the compound is from about 0.001 mg/kg/day to about 300 mg/kg/day.

28. The method of claim 24, wherein the method further comprises the use the compound for the manufacture of a medicament for treating, preventing or ameliorating an ORL-1 receptor mediated disorder and condition in a subject in need thereof.

29. A process for preparing a compound of any of claim 1 to 16 comprising the steps of 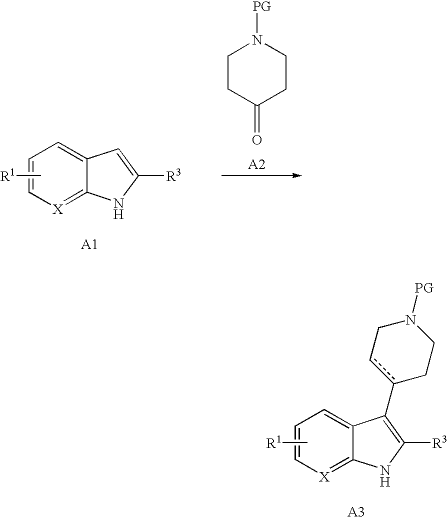
(a) reacting a Compound A1 with a Compound A2 in the presence of a base, wherein the base is present in an amount equal to or greater than about one molar equivalent of Compound A2, to provide a Compound A3; 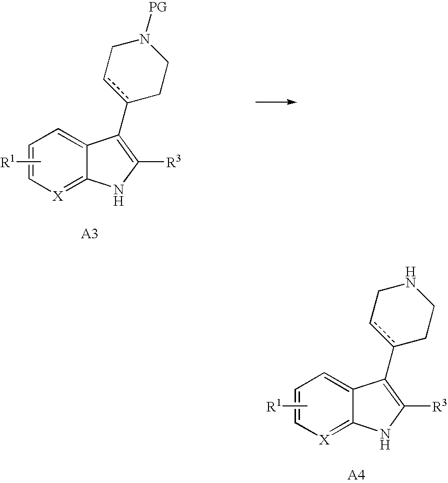
(b) deprotecting Compound A3 to provide a Compound A4; 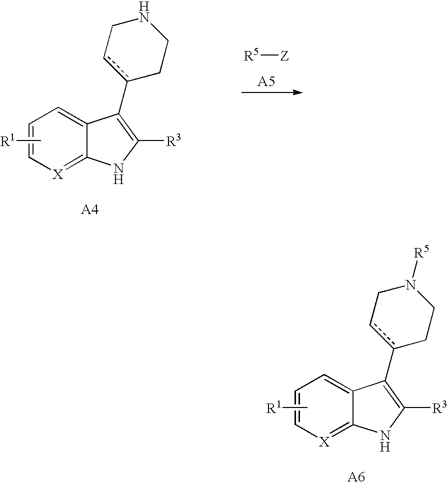
(c) reacting Compound A4 with a Compound A5 in the presence of a base, wherein the base is present in an amount equal to or greater than about one molar equivalent of Compound A5, to provide a Compound A6; and 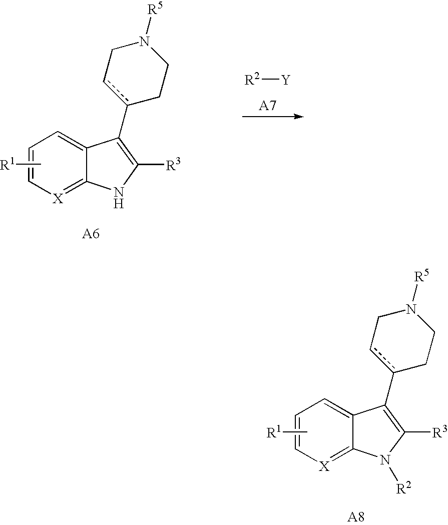
(d) reacting Compound A6 with a Compound A7 in the presence of a base, wherein the base is present in an amount equal to or greater than about one molar equivalent of Compound A7, to provide a Compound A8.

30. A process for preparing a compound of any of claim 1 to 16 comprising the steps of 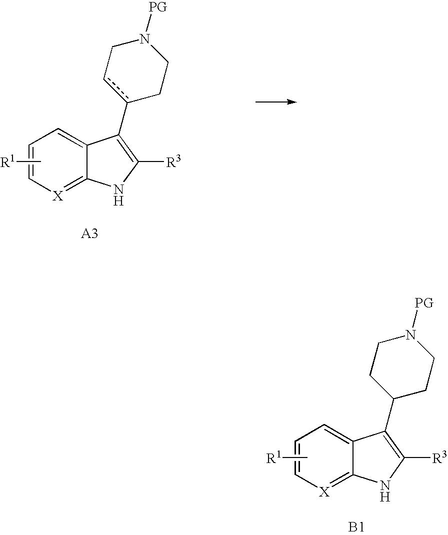
(a) reacting a Compound A3 in the presence of hydrogen blanket having a pressure in the range of from about 1 psi to about 60 psi at a temperature in the range of from about 20° C. to about 60° C. to provide a Compound B1;
(b) deprotecting Compound B1 to provide a deprotected Compound B1;
(c) reacting the deprotected Compound B1 with a Compound A5 in the presence of a base, wherein the base is present in an amount equal to or greater than about one molar equivalent of Compound A5, to provide an R⁵ substituted Compound B2; and
(d) reacting the R⁵ substituted Compound B2 with a Compound A7 in the presence of a base, wherein the base is present in an amount equal to or greater than about one molar equivalent of Compound A7, to provide an R² substituted Compound B3.

31. A process for preparing a compound of any of claim 1 to 16 comprising the steps of 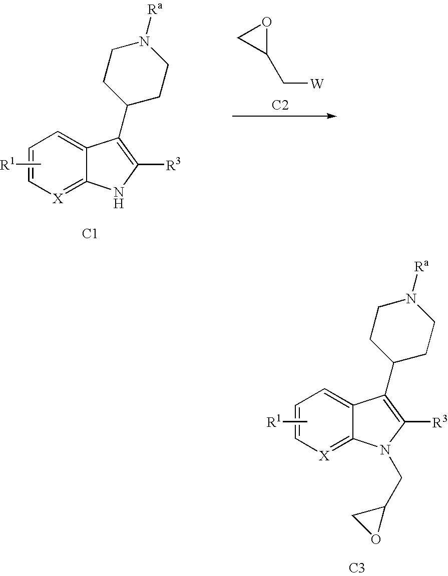
(a) reacting a Compound C1 (wherein R^{a} is a protecting group or R⁵) with a Compound C2 in the presence of a base to provide a Compound C3; and 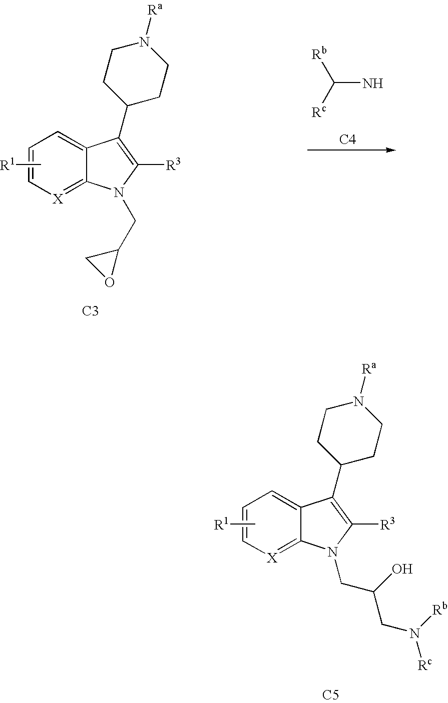
(b) reacting Compound C3 with a Compound C4 to yield a Compound C5.

32. A process for preparing a compound of any of claim 1 to 16 comprising the steps of 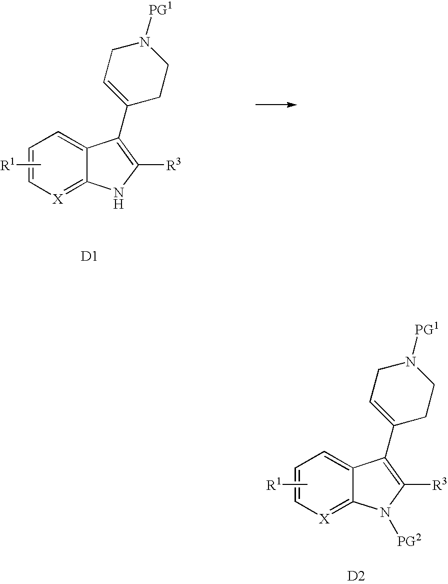
(a) reacting a Compound D1 with an anhydride in the presence of a base to provide a Compound D2; 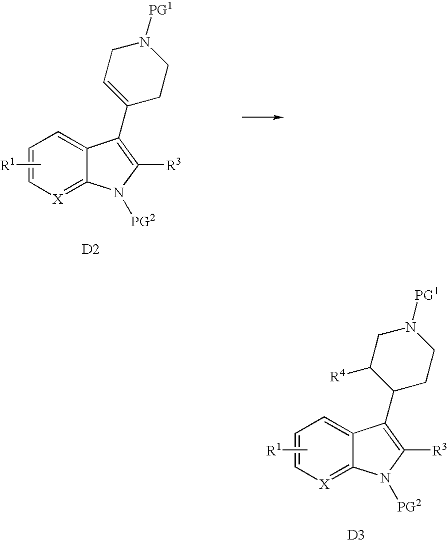
(b) reacting Compound D2 with a boranated reagent in the presence of a base to provide a Compound D3; or
(c) reacting Compound D2 with a catalyst, then reducing the product to yield a Compound D3; 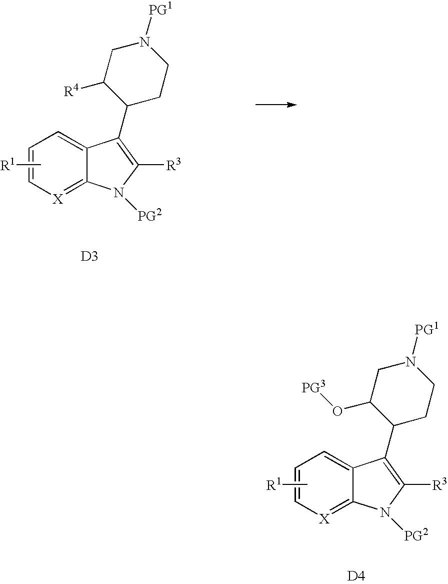
(d) reacting Compound D3 with an anhydride in the presence of a base to provide a Compound D4; 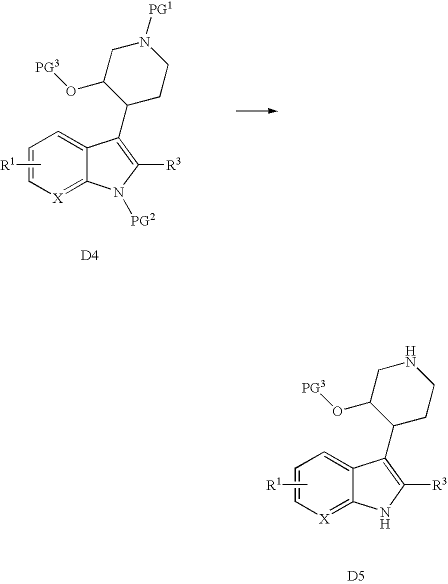
(e) deprotecting Compound D4 to provide a deprotected Compound D5; and 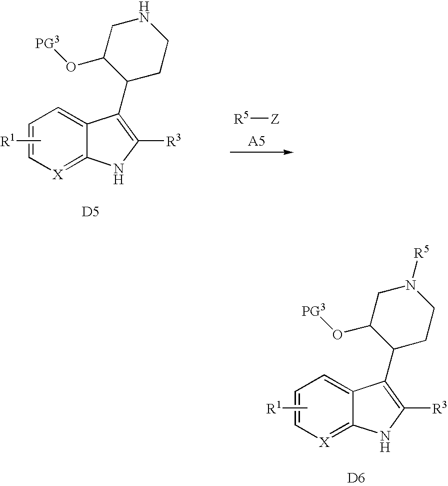
(f) reacting Compound D5 with a Compound A5 in the presence of a base, wherein the base is present in an amount equal to or greater than about one molar equivalent of Compound A5, to provide a Compound D6.
